(19) United States
(12) Patent Application Publication
(10) Pub. No.: US 2012/0014950 A1
(43) Pub. Date: Jan. 19, 2012

(54) Antibodies That Specifically Bind to DR3

(75) Inventors: Thi-Sau Migone, Gaithersburg, MD (US); Chih-Hung Lo, Rockville, MD (US); Luke Oh, Potomac, MD (US); Heather Wasserman, Gaithersburg, MD (US); Madhav Devalaraja, Rockville, MD (US); Matthew J. Bernett, Monrovia, CA (US)

(73) Assignees: Xencor, Inc., Monrovia, CA (US); Human Genome Sciences, Inc., Rockville, MD (US)

(21) Appl. No.: 13/217,023

(22) Filed: Aug. 24, 2011

Related U.S. Application Data
(63) Continuation-in-part of application No. PCT/US2011/026327, filed on Feb. 25, 2011.
(60) Provisional application No. 61/308,804, filed on Feb. 26, 2010.

(51) Int. Cl. A61K 39/395; A61P 29/00; A61P 37/02; A61P 19/10; A61P 1/00; A61P 25/00; A61P 37/08; A61P 35/00; C07K 16/00; A61P 19/02

(52) U.S. Cl. 424/133.1; 530/387.3

(57) ABSTRACT

The present invention relates to antibodies, antibody fragments, and related molecules that specifically bind to DR3 receptors. Such antibodies have uses, for example, in the prevention, detection, diagnosis, treatment or amelioration of a disease or disorder, especially inflammatory and autoimmune diseases and other immune system disorders, such as Crohn's disease, colitis, Inflammatory Bowel Disease, arthritis, asthma, Multiple Sclerosis, atherosclerosis, and allergic disorders. The invention also relates to nucleic acid molecules encoding anti-DR3 receptor antibodies, vectors and host cells containing these nucleic acids, and methods for producing the same. The present invention relates to methods and compositions for preventing, detecting, diagnosing, treating or ameliorating a disease or disorder, especially inflammatory or autoimmune disorders, comprising administering to an animal, preferably a human, an effective amount of one or more antibodies or fragments or variants thereof, or related molecules, that specifically bind to DR3 receptor.

### CROSS-REFERENCE TO RELATED APPLICATIONS

This application is a continuation-in-part of International Application No. PCT/US2011/026327, filed Feb. 25, 2011, which claims benefit under 35 U.S.C. §119(e) of U.S. Provisional Application No. 61/308,804, filed Feb. 26, 2010, each of which is hereby incorporated by reference in its entirety.

### REFERENCE TO SEQUENCE LISTING AS TEXT FIELD OF THE INVENTION

This application refers to a “Sequence Listing” listed below, which is provided as a text file. The text file contains a document entitled “PF622P1_SequenceListing.txt” (44,242 bytes, created Aug. 18, 2011), which is incorporated by reference in its entirety.

### FIELD OF THE INVENTION

The present invention relates to antibodies and related molecules that specifically bind to DR3 receptors. Such antibodies have uses, for example, in the prevention and treatment of inflammatory or autoimmune diseases, such as Crohn's disease, colitis, inflammatory bowel disease, arthritis, asthma, multiple sclerosis, diabetes, atherosclerosis, allergic disorders and osteoporosis; other immune system disorders; and bone cancer pain. The invention also relates to nucleic acid molecules encoding anti-DR3 receptor antibodies, vectors and host cells containing these nucleic acids, and methods for producing the same. The present invention relates to methods and compositions for preventing, detecting, diagnosing, treating or ameliorating a disease or disorder, especially inflammatory or autoimmune diseases, such as Crohn's disease, colitis, inflammatory bowel disease, arthritis, asthma, multiple sclerosis, diabetes, atherosclerosis, osteoporosis, and allergic disorders; other immune system disorders; and bone cancer pain comprising administering to an animal, preferably a human, an effective amount of one or more antibodies or fragments or variants thereof, or related molecules, that specifically bind to DR3 receptors.

### BACKGROUND OF THE INVENTION

Death Receptor 3 (DR3, also known as TNFRSF25, LARD, APO-3, TRAMP, and WSL-1) is a member of the TNF Receptor superfamily, and has been shown to be involved in the regulation of inflammation, homeostasis, autoimmunity and apoptosis (Migone et al. Immunity. 16:479-492. (2002)). DR3 is expressed primarily by T cells (Screaton et al., PNAS, 94:4615-4619 (1997); Su et al., PNAS 101:6062-6067 (2002)), and its interaction with TL1A can provide a costimulatory signal for the activation of T cells. Expression of DR3 is also observed on various other cell types including endothelial cells, epithelial cells, osteoblasts, and cells of monocytic lineage (Al-Lamki et al. Am. J. Pathol. 163:401-411 (2003); Kang et al. Cytokine 29:229-235 (2005); Borysenko et al. J. Cell Physiol. 209:1021-1028 (2006)).

TL1A (TNFSF15) is a member of the TNF Ligand superfamily and is a natural ligand of DR3. TL1A is expressed in endothelial cells as well as lymphocytes, plasma cells, and monocytes. Binding of TL1A to DR3 triggers proliferation and activation, through activation of NF-κB-mediated pathways (Migone, T., et al. Immunity. 16:479-492 (2002); Wen L. et al., J. Biol. Chem 278:39251-39258 (2003). TL1A enhances the secretion of IFN-γ and GM-CSF from T cells without affecting the production of IL-2, IL-4, IL-10, or TNF. (Migone et al. Immunity 16:479-492 (2002); Bamias et al. J. Immunol. 171:4868-4874 (2003); International Patent Publication WO99/23105).

DR3-TL1A signalling has been linked to several pathological conditions involving inflammation and autoimmunity. Intestinal tissue specimens from patients with inflammatory bowel disease (IBD) show overexpression of TL1A mRNA and protein, particularly in Crohn's disease and ulcerative colitis. Bamias et al. J Immunol. 171:4868-4874 (2003); see also Prehn et al. Clin Immunol. 112(1):66-77 (2004). The amount of TL1A overexpression is correlated with the severity of inflammation. Id. Moreover, increased numbers of DR3-positive T lymphocytes are detected in the intestinal lamina propria from IBD patients. Id. In addition, TL1A is overexpressed in synovial fluids and tissues obtained from rheumatoid arthritis (RA) patients who are rheumatoid factor (RF)— seropositive. Cassatella et al., J. Immunol. 178; 7325-7333 (2007). DR3 was shown to mediate apoptosis of osteoblasts and the TL-1A/DR3 interactions were shown to play a role in bone damage in inflammatory arthritis animal models (Bull et al. JEM. 205:2457-2464. (2008)). Evidence also indicates that TL1A-DR3 signalling contributes to atherosclerosis via the induction of pro-inflammatory cytokines Kang et al., Cytokine 29(5):229-35 (2005). Furthermore, genetic variants of TL1A and DR3 are associated with Crohn's disease and rheumatoid arthritis. Osawa et al., Genes Immun. 5:439-443 (2004); Yamazaki et al., Hum Mol Genet. 14:3499-3506 (2005).

Animal models also support the observed relationship between TL1A-DR3 signalling and inflammatory and autoimmune disorders. Bamias et al. demonstrated that TL1A and DR3 are upregulated in inflamed intestinal mucosa in mouse models of chronic ileitis. Proc Natl Acad Sci USA. 103(22):8441-6 (2006). In addition, DR3-deficient mice show decreased pathology in experimental autoimmune encephalomyelitis (EAE) and ova and alum-induced lung inflammation. Meylan et al., Immunity 29(1): 79-89 (2008); Fang et al., J Exp Med. 205(5):1037-48 (2008).

There is a clear need to identify antagonistic anti-DR3 antibodies or fragments thereof, that are capable of inhibiting DR3 signalling. Given the role of TL1A-DR3 signalling in numerous disorders such as inflammatory and autoimmune diseases, such antagonistic anti-DR3 antibodies or fragments thereof, have uses in the treatment and prevention of inflammatory and autoimmune disorders, including but not limited to Crohn's disease, colitis, inflammatory bowel disease, allergy, asthma, arthritis, atherosclerosis, osteoporosis, bone cancer pain, and multiple sclerosis.

### SUMMARY OF THE INVENTION

The present invention encompasses antibodies (including molecules comprising, or alternatively consisting of, antibody fragments or variants thereof) that specifically bind to a DR3 receptor polypeptide or polypeptide fragment or variant of a DR3 receptor. In particular, the invention encompasses antibodies (including molecules comprising, or alternatively consisting of, antibody fragments or variants thereof) that specifically bind to a polypeptide or polypeptide fragment or variant of human DR3 receptor (SEQ ID NO:2). The invention also encompasses antibodies (including molecules comprising, or alternatively consisting of, antibody fragments or variants thereof) that specifically bind to allelic variants of DR3 receptors. The invention also encompasses antibodies that specifically bind to DR3 receptors that are expressed on the surface of a cell.

Activation of the DR3 receptor by its cognate ligand, TL1A, is capable of inducing inflammatory responses such as T-cell activation, T-cell proliferation, T-cell infiltration, NF-κB activation and production of proinflammatory cytokines such as interferon gamma and GM-CSF. Thus, DR3 antagonists (e.g., antibodies or fragments thereof) that prevent TL1A binding to DR3 are useful in preventing such TL1A-mediated inflammatory responses.

Another function of the DR3 receptor is to induce programmed cell death by the association/cross-linking of DR3 death domains. Thus, agents (e.g., antibodies, fragments, or variants thereof) that prevent association/cross-linking of DR3 death domains will prevent DR3-mediated programmed cell death, and agents (e.g., antibodies) that induce association/cross-linking of DR3 death domains will induce DR3 mediated programmed cell death.

The present invention relates to methods and compositions for preventing, treating or ameliorating a disease or disorder comprising administering to an animal, preferably a human, an effective amount of one or more antibodies or fragments or variants thereof, or related molecules, that specifically bind to a DR3 receptor or a fragment or variant thereof. In highly preferred embodiments, the present invention relates to antibody fragments linked to a serum albumin polypeptide. In the most preferred embodiments, the present invention relates to a Fab fragment linked to a serum albumin polypeptide.

In specific embodiments, the present invention relates to methods and compositions for preventing, treating or ameliorating a disease or disorder associated with DR3 receptor function or DR3 receptor ligand function or aberrant DR3 receptor or DR3 receptor ligand expression, comprising administering to an animal, preferably a human, an effective amount of one or more antibodies or fragments or variants thereof, or related molecules, that specifically bind to a DR3 receptor or a fragment or variant thereof.

In highly preferred embodiments, the present invention related to antibodies and fragments or variants thereof, which are useful in the treatment and prevention of inflammatory and autoimmune disorders, including but not limited to Crohn's disease, colitis, inflammatory bowel disease, inflammatory bowel disease, allergy, asthma, arthritis, atherosclerosis, osteoporosis, bone cancer pain, and multiple sclerosis.

In highly preferred embodiments, the present invention relates to antagonist antibodies and fragments or variants thereof, which are capable of binding to a DR3 receptor and inhibiting DR3-mediated inflammatory responses. Such DR3-meditated inflammatory responses include, but are not limited to, T-cell activation, T-cell proliferation, T-cell infiltration, activation of cells of monocytic lineage, NF-κB activation, and production of proinflammatory cytokines such as interferon gamma and GM-CSF. In specific embodiments, antagonist antibodies of the present invention can be used to treat, prevent or ameliorate inflammatory disorders, including but not limited to arthritis (rheumatoid arthritis, osteoarthritis, septic arthritis, gout and pseudo-gout, juvenile idiopathic arthritis, Still's disease, and ankylosing spondylitis); asthma; allergic disorders; graft-versus-host disease; inflammatory bowel disease; ulcerative colitis; atherosclerosis; osteoporosis; bone cancer pain; and encephalomyelitis.

In additional preferred embodiments, antibodies of the invention can be used to treat, prevent or ameliorate an autoimmune disease (e.g., multiple sclerosis, rheumatoid arthritis, systemic lupus erhythematosus, idiopathic thrombocytopenia purpura, autoimmune hemolytic anemia, autoimmune neonatal thrombocytopenia, autoimmunocytopenia, hemolytic anemia, antiphospholipid syndrome, dermatitis, allergic encephalomyelitis, myocarditis, relapsing polychondritis, rheumatic heart disease, glomerulonephritis (e.g, IgA nephropathy), an immune-based rheumatologic disease (e.g., SLE, rheumatoid arthritis, CREST syndrome (a variant of scleroderma characterized by calcinosis, Raynaud's phenomenon, esophageal motility disorders, sclerodactyl), and telangiectasia.), Seronegative spondyloarthropathy (SpA), Polymyositis/dermatomyositis, Microscopic polyangiitis, Hepatitis C-associated arthritis, Takayasu's arteritis, and undifferentiated connective tissue disorder), Neuritis, Uveitis Ophthalmia, Polyendocrinopathies, Purpura (e.g., Henloch-Scoenlein purpura), Reiter's Disease, Stiff-Man Syndrome, Autoimmune Pulmonary Inflammation, Guillain-Barre Syndrome, insulin dependent diabetes mellitis, and autoimmune inflammatory eye, autoimmune thyroiditis, hypothyroidism (i.e., Hashimoto's thyroiditis, Goodpasture's syndrome, Pemphigus, Receptor autoimmunities such as, for example, (a) Graves' Disease, (b) Myasthenia Gravis, and (c) insulin resistance, autoimmune hemolytic anemia, autoimmune thrombocytopenic purpura, schleroderma with anti-collagen antibodies, mixed connective tissue disease, polymyositis/dermatomyositis, pernicious anemia, idiopathic Addison's disease, infertility, glomerulonephritis such as primary glomerulonephritis and IgA nephropathy, bullous pemphigoid, Sjogren's syndrome, diabetes millitus, and adrenergic drug resistance (including adrenergic drug resistance with asthma or cystic fibrosis), chronic active hepatitis, primary biliary cirrhosis, other endocrine gland failure, vitiligo, vasculitis, post-MI, cardiotomy syndrome, urticaria, atopic dermatitis, asthma, inflammatory myopathies, and other inflammatory, granulamatous, degenerative, and atrophic disorders) or conditions associated with an autoimmune disease. In a specific preferred embodiment, rheumatoid arthritis is treated, prevented, prognosed and/or diagnosed using antibodies of the invention.

In other preferred embodiments, the present invention relates to the use of antibodies and fragments or variants thereof, which are capable of binding to a DR3 receptor and activating DR3-mediated apoptosis. Such antibodies can be used for preventing, treating or ameliorating cancers and other hyperproliferative disorders (e.g., leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma).

The present invention also encompasses methods and compositions for detecting, diagnosing, or prognosing diseases or disorders comprising administering to an animal, preferably a human, an effective amount of one or more antibodies or fragments or variants thereof, or related molecules, that specifically bind to DR3 receptor or a fragment or variant thereof. In specific embodiments, the present invention also encompasses methods and compositions for detecting, diagnosing, or prognosing diseases or disorders associated with DR3 receptor function or DR3 receptor ligand function or aberrant DR3 receptor or DR3 receptor ligand expression, comprising administering to an animal, preferably a human, an effective amount of one or more antibodies or fragments or variants thereof, or related molecules, that specifically bind to DR3 receptor or a fragment or variant thereof. Other diseases and disorders which can be detected, diagnosed or prognosed with the antibodies of the invention include, but are not limited to, immune disorders (e.g., graft-versus-host disease, systemic lupus erythematosus, rheumatoid arthritis, multiple sclerosis, myasthenia gravis, and Hashimoto's disease), inflammatory disorders (e.g., asthma, allergic disorders, inflammatory bowel disease, ulcerative colitis, atherosclerosis, encephalomyelitis), and cancer.

Another embodiment of the present invention includes the use of the antibodies of the invention as a diagnostic tool to monitor the expression of DR3 receptor expression on cells.

The present inventors have generated a hybridoma cell line that produces an antibody that specifically binds DR3 receptor polypeptide (e.g., SEQ ID NO:2).

Further, the present invention encompasses the polynucleotides encoding the antibody expressed by this cell line, as well as the amino acid sequences encoding the antibody expressed by this cell line. Molecules comprising, or alternatively consisting of, fragments, variants, or derivatives of this antibody (e.g., heavy chains, VH domains, VH CDRs, light chains, VL domains, or VL CDRs having any one of the amino acid sequences referred to in Table 1), that specifically bind to a DR3 receptor or fragments or variants thereof are also encompassed by the invention, as are nucleic acid molecules that encode these antibodies and/or molecules. In highly preferred embodiments, the present invention encompasses antibodies, or fragments or variants thereof, that bind to the extracellular regions/domains of DR3 receptors or fragments and variants thereof.

The present invention also provides antibodies and fragments or variants thereof that bind DR3 receptor polypeptides which are coupled to a detectable label, such as an enzyme, a fluorescent label, a luminescent label, or a bioluminescent label. The present invention also provides antibodies and fragments or variants thereof that bind DR3 receptor polypeptides which are coupled to a therapeutic or cytotoxic agent. The present invention also provides antibodies and fragments or variants thereof that bind DR3 receptor polypeptides which are coupled to a radioactive material.

The present invention also provides for fusion proteins comprising an antibody (including molecules comprising, or alternatively consisting of, antibody fragments or variants thereof) of the invention, and a heterologous polypeptide (i.e., a polypeptide unrelated to an antibody or antibody domain). Nucleic acid molecules encoding these fusion proteins are also encompassed by the invention. A composition of the present invention may comprise, or alternatively consist of, one, two, three, four, five, ten, fifteen, twenty or more fusion proteins of the invention. Alternatively, a composition of the invention may comprise, or alternatively consist of, nucleic acid molecules encoding one, two, three, four, five, ten, fifteen, twenty or more fusion proteins of the invention. In other specific embodiments, the antibodies of the invention inhibit DR3 receptor ligand (e.g. TL1A) binding to a DR3 receptor. In highly preferred embodiments, antibodies or fragments thereof of the invention are genetically fused to a serum albumin polypeptide, such as human serum albumin (HSA).

The present invention also provides antibodies that bind one or more DR3 receptor polypeptides that act as either DR3 receptor agonists or DR3 receptor antagonists.

In specific embodiments, the antagonist antibodies and fragments or variants thereof of the invention inhibit proliferation of DR3 receptor expressing cells. In specific embodiments, the antagonist antibodies of the invention inhibit activation and proliferation of DR3 receptor expressing cells (e.g., T-cells). In specific embodiments, the antagonist antibodies of the invention inhibit DR3-mediated co-stimulation of T-cell activation. In other specific embodiments, the antagonist antibodies of the invention inhibit T-cell infiltration. In other specific embodiments, the antagonist antibodies of the invention inhibit inflammatory cytokine secretion. In a specific embodiment, the antagonist antibodies of the invention inhibit interferon gamma (IFN-γ) secretion. In another specific embodiment, the antagonist antibodies of the invention inhibit Granulocyte Colony Macrophage Stimulating Factor (GCMSF) secretion. In another specific embodiment, the antagonist antibodies of the invention inhbit DR3-mediated apoptotis of DR3 receptor expressing cells.

In other specific embodiments, the agonist antibodies and fragments or variants thereof, of the invention stimulate activation and proliferation of DR3 receptor expressing cells (e.g., T-cells, monocytes, etc.). In specific embodiments, the agonist antibodies of the invention stimulate DR3-mediated co-stimulation of T-cell activation. In other specific embodiments, the agonist antibodies of the invention stimulate DR3 receptor ligand binding to a DR3 receptor. In other specific embodiments, the agonist antibodies of the invention stimulate T-cell infiltration. In other specific embodiments, the agonist antibodies of the invention stimulate inflammatory cytokine secretion. In a specific embodiment, the agonist antibodies of the invention stimulate IFN-γ secretion. In other specific embodiments, the agonist antibodies of the invention stimulate GM-CSF secretion. In other specific embodiments, the agonist antibodies of the invention stimulate DR3-mediated apoptotis of DR3 receptor expressing cells.

In further embodiments, the antibodies of the invention have a dissociation constant (KD) of 10⁻⁶ M or less. In further embodiments, the antibodies of the invention have a dissociation constant (KD) of 10⁻⁷ M or less. In preferred embodiments, the antibodies of the invention have a dissociation constant (KD) of 10⁻⁹ M or less.

The present invention further provides antibodies and fragments or variants thereof, which inhibit activation and proliferation of DR3 receptor expressing cells better than an equal concentration of a soluble DR3 polypeptide.

The present invention further provides antibodies that stimulate proliferation of DR3 receptor expressing cells equally well in the presence or absence of antibody cross-linking reagents; and/or stimulate proliferation with equal or greater potency as an equal concentration of DR3 in the absence of a cross-linking antibody or other cross-linking agent.

In further embodiments, antibodies of the invention have an off rate (koff) of 10-3/sec or less. In preferred embodiments, antibodies of the invention have an off rate (koff) of 10-4/sec or less. In other preferred embodiments, antibodies of the invention have an off rate (koff) of 10-5/sec or less.

The present invention also provides panels of antibodies (including molecules comprising, or alternatively consisting of, antibody fragments or variants) wherein the panel members correspond to one, two, three, four, five, ten, fifteen, twenty, or more different antibodies of the invention (e.g., whole antibodies, Fabs, F(ab′)2 fragments, Fd fragments, disulfide-linked Fvs (sdFvs), anti-idiotypic (anti-Id) antibodies, and scFvs). The present invention further provides mixtures of antibodies, wherein the mixture corresponds to one, two, three, four, five, ten, fifteen, twenty, or more different antibodies of the invention (e.g., whole antibodies, Fabs, F(ab′)2 fragments, Fd fragments, disulfide-linked Fvs (sdFvs), anti-idiotypic (anti-Id) antibodies, and scFvs)). The present invention also provides for compositions comprising, or alternatively consisting of, one, two, three, four, five, ten, fifteen, twenty, or more antibodies of the present invention (including molecules comprising, or alternatively consisting of, antibody fragments or variants thereof). A composition of the invention may comprise, or alternatively consist of, one, two, three, four, five, ten, fifteen, twenty, or more amino acid sequences of one or more antibodies or fragments or variants thereof. Alternatively, a composition of the invention may comprise, or alternatively consist of nucleic acid molecules encoding one or more antibodies, fragments or variants thereof, of the invention.

The present invention also provides for a nucleic acid molecule(s), generally isolated, encoding an antibody (including molecules, such as scFvs, VH domains, or VL domains, that comprise, or alternatively consist of, an antibody fragment or variant thereof) of the invention. The present invention also provides a host cell transformed with a nucleic acid molecule of the invention and progeny thereof. The present invention also provides a method for the production of an antibody (including a molecule comprising, or alternatively consisting of, an antibody fragment or variant thereof) of the invention. The present invention further provides a method of expressing an antibody (including a molecule comprising, or alternatively consisting of, an antibody fragment or variant thereof) of the invention from a nucleic acid molecule. These and other aspects of the invention are described in further detail below.

### BRIEF DESCRIPTION OF THE FIGURES

FIGS. 1A and 1B. FIG. 1A shows the amino acid sequence alignment of the VH domain of murine 11H08 monoclonal antibody (SEQ ID NO:7) with the VH domains of humanized variant antibody fragments (11H08_H1, 11H08_H2, 11H08_H3, 11H08_H4, 11H08_H5; SEQ ID NOs:9, 11, 13, 15, and 17, respectively). The figure depicts both Kabat numbering and sequential amino acid numbering. The shaded regions show the CDRs of the antibody fragments. VHCDR1 comprises amino acid residues 31 to 35 (Kabat positions 31 to 35), VHCDR2 comprises amino acid residues 50 to 66 (Kabat positions 50 to 65), and VHCDR3 comprises amino acid residues 99 to 110 (Kabat positions 95 to 102). FIG. 1B shows the alignment of the amino acid sequences of additional humanized variants (11H08_H0.1, 11H08_H1.1, 11H08_H2.1, 11H08_H3.1, 11H08_H4.2, 11H08_H5.2, and 11H08_H5.3; SEQ ID NOs: 8, 10, 12, 14, 16, 18, and 19, respectively). For example, H0 was further altered at Kabat position 91 (amino acid position 95) by replacing an asparagine with a tyrosine to produce H0.1. The heavy chains of the invention are referred to herein both in relation to the 11H08 murine hybridoma (e.g., 11H08_H1) and by the heavy chain designation alone (e.g., H1); these designations are equivalent.

FIG. 2. Amino acid sequence alignment of the VL domain of murine 11H08 monoclonal antibody (SEQ ID NO:20) with the VL domains of humanized variant antibody fragments (SEQ ID NOs:21-24). The figure depicts both Kabat numbering and sequential amino acid numbering. The shaded regions show the CDRs of the antibody fragments. The VLCDR1 comprises amino acid residues 24-38 (Kabat positions 24 to 34), VLCDR2 comprises amino acid residues 54 to 60 (Kabat positions 50 to 56) and VLCDR3 comprises amino acid residues 93 to 101 (Kabat positions 89 to 97). The light chains of the invention are referred to herein both in relation to the 11H08 murine hybridoma (e.g., 11H08_L1) and by the heavy chain designation alone (e.g., L1); these designations are equivalent.

FIG. 3. Representation of a full-length immunoglobulin molecule (IgG1) consisting of a Fab fragment containing the VL, VH, CH1 and Cκ domains, and the Fc fragment containing the constant regions. This figure also includes a representation of the heavy-chain HSA (HC-HSA) molecule consisting of a Fab fragment with the carboxy-terminus of the CH1 domain of the heavy chain fused to a human serum albumin (HSA) polypeptide.

FIG. 4. Flow cytometry results of 11H08 binding to hDR3-expressing HEK293F cells. HEK293F cells transiently expressing human DR3 were used to test the ability of the whole 11H08 immunoglobulin molecule to inhibit human TL1A binding to DR3 (See, Example 2). HEK293F cells were incubated with 1 μg/mL of human TL1A in the presence of either an unrelated antibody (mIgG1) as an isotype control (known not to bind TL1A or DR3) or 11H08. Flow cytometry results show that the full-length anti-DR3 monoclonal antibody 11H08 inhibited binding of TL1A to DR3 expressed on the cell surface. The upper panels show binding of labeled TL1A either by itself or in the presence of an unrelated antibody (mIgG1). The bottom panel shows that addition of 11H08 inhibited binding of TL1A to DR3.

FIG. 5. Agonist activity of the whole 11H08 antibody. Example 3 describes how TF1 cells were treated with media and cycloheximide along with varying concentrations of the whole 11H08 immunoglobulin or 10 ng/mL of the positive control hTL1A protein, as indicated. This was compared with cells treated only with media and cycloheximide. Full length anti-DR3 antibodies act as TL1A agonists and induce cell death.

FIGS. 6A and B. Pharmacokinetics of mouse surrogate anti-DR3 molecules. As described in Example 7, serum concentrations of the anti-DR3 molecules were taken at the specified time points following a 1 mg/kg intravenous injection of the indicated molecules into Balb/c mice. The addition of MSA to the D06-Fab molecule, significantly improved the half-life of the Fab.

FIG. 7. D06-ScFv increases proliferation of murine T cells. As described in Example 8, murine CD4+ T-cells were treated with varying concentrations of mouse surrogate D06-scFv with or without 10 ng/mL mTL1A. Cells were also treated with media alone, 10 ng/mL mTL1A, and 1 μg/mL TACI-Fc plus 10 ng/mL mTL1A as controls as indicated. D06-scFv had an agonistic effect on proliferation of murine T cells. Even in the absence of mTL1A, treatment with D06-scFv increased proliferation, as measured by luminescence. The addition of 10 ng/mL to the lower concentrations of D06-scFv (0.11 μg/mL and 0.33 μg/mL) resulted in even greater proliferation of the murine T cells.

FIG. 8. Anti-DR3 Fab blocks TL1A-induced cell death. As described in Example 9, TF-1 cells were treated with 10 ng/mL hTL1A and cycloheximide, along with 1 μg/mL 11H08-Fab, 1 μg/mL mIgG1 (unrelated negative control antibody), or with various concentrations of 11H08-scFv-HSA as indicated. Cells were also treated with cycloheximide alone and 10 ng/mL hTL1A alone as further controls as indicated. The addition of hTL1A and cycloheximide to the cells caused a decrease in TF-1 cell viability (measured by luminescence) that was not blocked by the addition of any of the concentrations of the 11H08-scFv-HSA molecule. However, the 11H08-Fab molecule, which also binds DR3 receptor, blocked TL1A activity in this assay.

FIG. 9. 11H08-Fab molecules block TL1A-induced cell death. As described in Example 9, TF-1 cells were treated with 10 ng/mL hTL1A and cycloheximide, along with varying concentrations of the anti-DR3 Fab molecules H1L2-Fab, H5L2-Fab and 11H08-Fab, as indicated. As shown by the graph, H1L2-Fab, H5L2-Fab and 11H08-Fab inhibited TL1A-induced apoptosis in human TF-1 cells, showing a dose-dependent prevention of apoptosis in cycloheximide-treated TF-1 cells in the presence of TL1A.

FIG. 10. Anti-DR3 Fab-HSA molecules block TL1A-induced cell death. As described in Example 9, TF-1 cells were treated with 10 ng/mL hTL1A and cycloheximide, along with varying concentrations of the anti-DR3 Fab, HSA fusion molecules H1L2-Fab-HSA, H5L2-Fab-HSA and 11H08-Fab-HSA, as indicated. This graph shows that H1L2-Fab-HSA, H5L2-Fab-HSA and 11H08-Fab-HSA molecules inhibited TL1A-induced apoptosis in human TF-1 cells, showing a dose-dependent prevention of apoptosis in cycloheximide-treated TF-1 cells in the presence of TL1A.

FIG. 11. Anti-DR3 Fab-HSA molecules block TL1A-induced cell death. Summary of results from human TF-1 proliferation assays, as described in Example 9, showing that various anti-DR3-Fab-HSA molecules (H5L2-Fab-HSA, H1L2-Fab-HSA, H1L4-Fab-HSA, H2L2-Fab-HSA, H2L4-Fab-HSA, and 11H08-Fab-HSA) protected human TF-1 cells from TL1A-induced apoptosis in a dose-dependent manner. The dashed line across the graph indicates the baseline level of apoptosis induced by treatment with cycloheximide and TL1A with no anti-DR3 antibody molecules added. All of the molecules tested showed greater cell protection from apoptosis than this baseline.

FIG. 12. Anti-DR3 Fab-HSA molecules block TL1A signaling. The effects of 10 μg/mL H1L2-Fab-HSA (AB13350-C1) or H5L2-Fab-HSA (AB13351-C1) on NF-κB activity were tested using a SEAP assay in TF1 cells as described in Example 11. The TF1 cells were also treated with either 100 ng/mL or 1 μg/mL of hTL1A, as indicated. Controls were run with no additional antibody, 10 μg/mL DR3-Fc, as indicated. A set of cells was also treated with only 10 ng/mL hTL1A. Both H1L2-Fab-HSA and H5L2-Fab-HSA inhibited TL1A signaling through NF-κB at both hTL1A concentrations tested. Inhibition was comparable to the DR3-Fc control.

FIG. 13. Anti-DR3 Fab-HSA molecules block TL1A-induced cell proliferation. As described in Example 13, primary T cells were treated with 100 ng/mL hTL1A alone or in combination with 3 μg/mL of either 11H08-Fab-HSA or hDR3-Fc. A set of cells was also tested with 3 μg/mL 11H08 whole immunoglobulin molecule alone. 11H08-Fab-HSA and hDR3-Fc inhibited TL1A-induced cell proliferation of these cells. Conversely, the whole 11H08 immunoglobulin molecule induced TL1A-like activity.

FIG. 14. Anti-DR3 Fab-HSA molecules block TL1A-induced cytokine release. As described in Example 12, primary T cells were treated with 100 ng/mL hTL1A alone or in combination with 3 μg/mL of either 11H08-Fab-HSA or hDR3-Fc. A set of cells was also tested with 3 μg/mL 11H08 whole immunoglobulin molecule alone. The assay measured release of both human IFN-γ and GM-CSF. 11H08-Fab-HSA and hDR3-Fc inhibited TL1A-induced cytokine secretion in these cells, for both IFN-γ and GM-CSF. Conversely, the whole 11H08 immunoglobulin molecule induced TL1A-like activity.

FIG. 15A. D06-Fab-MSA treatment reduces arthritis symptoms in the murine collagen induced arthritis (CIA) model. As described in Example 13, mice with CIA were treated prophylactically with 200 μg intraperitoneal (i.p.) of D06-Fab-MSA or anti-TNF-α antibody. Control sets of mice with CIA were either left untreated or received 50 μg/mL dexamethasone. D06-Fab-MSA significantly reduced the symptoms of arthritis in the mouse model compared with the untreated CIA-mice.

FIG. 15B. Treatment with 2D9-Fab-MSA or 5D10-Fab-MSA reduces arthritis symptoms in the murine collagen induced arthritis (CIA) model. As described in Example 13, mice with CIA were treated prophylactically with 200 μg intraperitoneal (i.p.) of either of these therapeutics or an isotype control antibody (“IgG1”). Both 2D9-Fab-MSA and 5D10-Fab-MSA significantly reduced the symptoms of arthritis in the mouse model compared with the untreated CIA-mice. FIG. 16A. The joints of untreated CIA-mice demonstrated significant bone erosion compared to the joints of mice without CIA. This figure shows bone erosion in the murine collagen induced arthritis (CIA) model, as described in Example 13, from paws from untreated mice with CIA (upper panel) and mice without CIA (lower panel). The paws were stained with trichrome stain to detect bone erosion. Blue staining indicates that there is no bone erosion; red staining indicates bone erosion. The original color images were converted to gray-scale for purposes of presentation. On the modified images, black has replaced blue staining which indicates that there is no bone erosion; light gray has replaced red staining which indicates bone erosion. As expected, the mice without CIA showed no significant bone erosion while the bone erosion in the joints of the untreated CIA-mice is pronounced.

FIGS. 16A and B. The joints of D06-Fab-MSA treated CIA-mice did not demonstrate significant bone erosion compared to the joints of mice without CIA. This figure shows bone erosion in a murine CIA model, as described in Example 13, from paws from mice treated with D06-Fab-MSA that had a clinical score of 4 (upper panel) and mice treated with D06-Fab-MSA that had a clinical score of 0 (lower panels). The paws were stained with trichrome stain to detect bone erosion, as indicated for FIGS. 16A and 16B. As indicated in Table 5, the score is a measurement of clinical symptoms in the animal, with a maximum score of 4 per paw. A score of 0 indicates a normal paw, while a score of 4 indicates severe clinical symptoms of arthritis. The two top panels show the paws of CIA-mice treated with D06-Fab-MSA which demonstrated clinical manifestations of arthritis (score=4), but did not show the significant bone erosion in the joints observed in the untreated CIA-mice (See, FIG. 16A). The bottom panels show the paws of CIA-mice treated with D06-Fab-MSA which demonstrated no clinical manifestations of arthritis (score=0) and did not show significant bone erosion in the joints.

FIG. 17. There was no discernable difference in terminal collagen antibody serum levels from CIA mice treated with different therapies. As described in Example 14, the serum levels of anti-collagen antibodies did not significantly vary depending upon treatment. The CIA mice all showed increased systemic levels of anti-collagen antibodies regardless of treatment (D06-Fab-MSA, isotype control, anti-mTNF-α antibody, or dexamethasone) compared with naïve mice.

FIG. 18. D06-Fab-MSA treatment in the MOG-EAE murine model of multiple sclerosis reduced the severity of clinical symptoms. As described in Example 15, mice with MOG-EAE were intravenously treated prophylactically and/or therapeutically with 10 mg/kg of a negative control antibody (does not bind DR3), D06-Fab-MSA, or mDR3-Fc, as indicated. Therapeutic treatment with D06-Fab-MSA suppressed the severity of clinical EAE symptoms in the murine MOG-EAE model.

FIG. 19. D06-Fab-MSA treatment in the MOG-EAE murine model of multiple sclerosis reduced the severity of clinical symptoms. As described in Example 15, mice with MOG-EAE were intravenously treated therapeutically with 25 mg/kg of D06-Fab-MSA or mDR3-Fc, as indicated. Therapeutic treatment with D06-Fab-MSA reduced the onset and severity of clinical EAE symptoms in the murine MOG-EAE model.

FIG. 20. The D06-Fab-MSA therapeutic molecule was detected and sustained its systemic level D06-Fab-MSA in the MOG-EAE murine model of multiple sclerosis (as described in Example 15) over 6 days.

FIGS. 21A and B. 2D9-Fab-MSA treatment reduced bone erosion in the murine collagen induced arthritis (CIA) model. As described in Example 13, Micro CT scans and densitometry show that paws from mice treated with the isotype control (IgG1) had significantly more cortical roughness when compared to mice treated with 2D9-Fab-MSA.

FIGS. 22A and B. 2D9-Fab-MSA treatment reduced bone erosion in the murine collagen induced arthritis (CIA) model. As described in Example 13, Micro CT scans and densitometry show that knees from mice treated with the isotype control (IgG1) had significantly more cortical roughness when compared to mice treated with 2D9-Fab-MSA.

FIG. 23. 2D9-Fab-MSA treatment protects mice from weight loss in the murine DSS-induced colitis model. As described in Example 16, this figure shows that IV administration of 2D9-Fab-MSA (10 mg/kg) on days 0 and 6 protected mice from weight loss over 10 days. The DSS-colitis mice treated with 2D9-Fab-MSA lost significantly less weight than the untreated DSS-colitis mice. The group treated with 2D9-Fab-MSA maintained their body weight through day 7 and lost only 7% of their initial body weight by day 9, compared to the untreated group which lost 13% of their initial body weight by day 9.

FIGS. 24A and B: 2D9-Fab-MSA treatment affects the concentration of serum cytokines in the DSS-colitis model. As described in Example 16, serum levels of IL-10, IL-4, CXCL-1, and TNF-α were increased in animals with DSS-induced colitis compared with the naïve mice. Treatment with 2D9-Fab-MSA resulted in levels of IL-10 and TNF-α about 30% lower than in the Untreated animals. Outliers beyond the range of 2×SD were excluded in the analysis based on Chauvenet's criterion (n=7-9).

FIGS. 25A and B: 2D9-Fab-MSA treatment affects the production of gut cytokines in the DSS-colitis model. As described in Example 16, all nine cytokines were elevated 3 to 50 fold in mice with colitis compared to the naïve group. Treatment of the DSS-colitis mice with 2D9-Fab-MSA reduced most of the Th1/Th2 cytokines tested except IL-2. Reduction of cytokine levels ranged from 17% to 75% in response to 2D9-Fab-MSA treatment. Outliers beyond the range of 2×SD were excluded in the analysis based on Chauvenet's criterion (n=8-9).

FIG. 26: 2D9-Fab-MSA treatment protected mice from weight loss in the adoptive transfer colitis murine model. As described in Example 17, weekly IV administration of 2D9-Fab-MSA (10 mg/kg) starting from 2.5 weeks protected mice from weight loss over 7.5 weeks, whereas administration of the isotype control, AN02-Fab-MSA, (10 mg/kg) did not prevent colitis progression and weight loss.

FIGS. 27A and B: 2D9-Fab-MSA treatment affects the production of serum cytokines in the adoptive transfer colitis murine model. As described in Example 17, serum levels of all cytokines tested, except IL-5, were increased in animals with adoptive transfer colitis. Particularly, the IFN-γ and IL-12 levels of the animals with adoptive transfer colitis were four and two fold greater, respectively, than in naïve mice. Weekly IV administration of 2D9-Fab-MSA (10 mg/kg) reduced the serum levels of IL-10, IL-12, CXCL-1 and TNF-α compared to the isotype control (AN02-Fab-MSA) treated group. Outliers beyond the range of 2×SD were excluded in the analysis based on Chauvenet's criterion (n=8-9).

### DETAILED DESCRIPTION OF THE INVENTION

### Definitions

The term “antibody,” as used herein, refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments as well as variants (including derivatives) of antibodies and antibody fragments. Examples of molecules which are described by the term “antibody” herein include, but are not limited to: single chain Fvs (sdFvs), Fab fragments, Fab′ fragments, F(ab′)2, disulfide linked Fvs (sdFvs), Fvs, and fragments comprising or alternatively consisting of, either a VL or a VH domain. The term “single chain Fv” or “scFv” as used herein refers to a polypeptide comprising a VH domain of antibody linked to a VL domain of an antibody. The antibodies of the invention may further comprise a heterologous polypeptide, detectable label, or other molecule. Antibodies that specifically bind to a DR3 receptor may have cross-reactivity with other antigens. Preferably, antibodies that specifically bind to a DR3 receptor do not cross-react with other antigens (e.g., other members of the Tumor Necrosis Factor Receptor superfamily). Antibodies that specifically bind to a DR3 receptor can be identified, for example, by immunoassays or other techniques known to those of skill in the art, e.g., immunoassays.

Antibodies of the invention include, but are not limited to, monoclonal, multispecific, humanized, human or chimeric antibodies, single chain antibodies, Fab fragments, F(ab′) fragments, anti-idiotypic (anti-Id) antibodies (including, e.g., anti-Id antibodies to antibodies of the invention), intracellularly-made antibodies (i.e., intrabodies), and epitope-binding fragments of any of the above. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Preferably, an antibody of the invention comprises, or alternatively consists of, a VH domain, VH CDR, VL domain, or VL CDR having an amino acid sequence of any one of those referred to in Table 1, or a fragment or variant thereof. In a preferred embodiment, the immunoglobulin is an IgG1 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda forms. In another preferred embodiment, the antibody of the invention comprises a Fab fragment fused to a heterologous polypeptide.

The term “variant” as used herein refers to a polypeptide that possesses a similar or identical function as a an anti-DR3 receptor antibody or antibody fragment thereof, but does not necessarily comprise a similar or identical amino acid sequence of an anti-DR3 receptor antibody or antibody fragment thereof, or possess a similar or identical structure of an anti-DR3 receptor antibody or antibody fragment thereof. A variant having a similar amino acid sequence refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence an anti-DR3 receptor antibody or antibody fragment thereof (including a VH domain, VHCDR, VL domain, or VLCDR having an amino acid sequence of any one of those referred to in Table 1) described herein; (b) a polypeptide encoded by a nucleotide sequence, the complementary sequence of which hybridizes under stringent conditions to a nucleotide sequence encoding a an anti-DR3 receptor antibody or antibody fragment thereof (including a VH domain, VHCDR, VL domain, or VLCDR having an amino acid sequence of any one of those referred to in Table 1), described herein, of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99%, identical to the nucleotide sequence encoding an anti-DR3 receptor antibody or antibody fragment thereof (including a VH domain, VHCDR, VL domain, or VLCDR having an amino acid sequence of any one of those referred to in Table 1), described herein. A polypeptide with similar structure to an anti-DR3 receptor antibody or antibody fragment thereof, described herein refers to a polypeptide that has a similar secondary, tertiary or quaternary structure of an anti-DR3 receptor antibody, or antibody fragment thereof, described herein. The structure of a polypeptide can determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268 (1990), modified as in Karlin and Altschul Proc. Natl. Acad. Sci. USA 90:5873-5877 (1993). The BLASTn and BLASTx programs of Altschul, et al. J. Mol. Biol. 215:403-410 (1990) have incorporated such an alogrithm. BLAST nucleotide searches can be performed with the BLASTn program (score=100, wordlength=12) to obtain nucleotide sequences homologous to a nucleic acid molecules of the invention. BLAST protein searches can be performed with the BLASTx program (score=50, wordlength=3) to obtain amino acid sequences homologous to a protein molecules of the invention. To obtain gapped alignments for comparison purposes, Gapped BLAST can be utilized as described in Altschul et al. Nucleic Acids Res. 25:3589-3402 (1997). Alternatively, PSI-BLAST can be used to perform an iterated search which detects distant relationships between molecules (Id.). When utilizing BLAST, Gapped BLAST, and PSI-BLAST programs, the default parameters of the respective programs (e.g., BLASTx and BLASTn) can be used. (National Center for Biotechnology Information, National Institutes of Health, Bethesda, Md.).

Another example of a mathematical algorithm utilized for the comparison of sequences is the algorithm of Myers and Miller, CABIOS (1989). The ALIGN program (version 2.0) which is part of the GCG sequence alignment software package has incorporated such an alogrithm. Other algorithms for sequence analysis known in the art include ADVANCE and ADAM as described in Torellis and Robotti Comput. Appl. Biosci., 10:3-5 (1994); and FASTA described in Pearson and Lipman Proc. Natl. Acad. Sci. 85:2444-8 (1988). Within FASTA, ktup is a control option that sets the sensitivity and speed of the search.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

By a polypeptide having an amino acid sequence at least, for example, 95% “identical” to a reference amino acid sequence of a DR3 polypeptide is intended that the amino acid sequence of the polypeptide is identical to the reference sequence except that the polypeptide sequence may include up to five amino acid alterations per each 100 amino acids of the reference amino acid of the DR3 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:2 can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The term “derivative” as used herein, refers to a variant polypeptide of the invention that comprises, or alternatively consists of, an amino acid sequence of an antibody or fragment thereof of the invention that specifically binds to a DR3 receptor polypeptide, which has been altered by the introduction of amino acid residue substitutions, deletions or additions. The term “derivative” as used herein also refers to an antibody or fragment thereof that specifically binds to a DR3 receptor polypeptide which has been modified, e.g., by the covalent attachment of any type of molecule to the polypeptide. For example, but not by way of limitation, an anti-DR3 receptor antibody or fragment thereof, may be modified, e.g., by glycosylation, acetylation, pegylation, phosphorylation, amidation, derivatization by known protecting/blocking groups, proteolytic cleavage, linkage to a cellular ligand or other protein, etc. A derivative of an anti-DR3 receptor antibody or fragment thereof, may be modified by chemical modifications using techniques known to those of skill in the art, including, but not limited to, specific chemical cleavage, acetylation, formylation, metabolic synthesis of tunicamycin, etc. Further, a derivative of an anti-DR3 receptor antibody or fragment thereof, may contain one or more non-classical amino acids. A polypeptide derivative possesses a similar or identical function as an anti-DR3 receptor antibody or fragment thereof, described herein.

The term “epitopes” as used herein refers to portions of DR3 receptor having antigenic or immunogenic activity in an animal, preferably a mammal. An epitope having immunogenic activity is a portion of DR3 receptor that elicits an antibody response in an animal. An eptiope having antigenic activity is a portion of DR3 receptor to which an antibody specifically binds as determined by any method known in the art, for example, by the immunoassays described herein. Antigenic epitopes need not necessarily be immunogenic.

The term “fragment” as used herein refers to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of an anti-DR3 receptor antibody (including molecules such as scFvs or Fabs, that comprise, or alternatively consist of, antibody fragments or variants thereof) that specifically binds to DR3 receptor.

The term “fusion protein” as used herein refers to a polypeptide that comprises, or alternatively consists of, an amino acid sequence of an anti-DR3 receptor antibody of the invention or fragment or variant thereof and an amino acid sequence of a heterologous polypeptide (i.e., a polypeptide unrelated to an antibody or antibody domain).

By DR3 receptor “agonist” is intended naturally occurring and synthetic compounds capable of enhancing or potentiating DR3 mediated biological activities such as cellular proliferation and/or differentiation. By DR3 receptor “antagonist” is intended naturally occurring and synthetic compounds, including antibodies and fragements or variants thereof, capable of inhibiting ligand binding to the DR3 receptor and/or DR3 mediated biological activities such as cellular proliferation, activation, and/or cytokine secretion. Whether any candidate “agonist” or “antagonist” of the present invention can enhance or inhibit, respectively, cellular proliferation, activation, and/or cytokine secretion can be determined using art-known TNF-family ligand/receptor cellular response assays, including those described in more detail below.

The term “host cell” as used herein refers to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

Unless indicated, “DR3 proteins”, “DR3 receptors”, “DR3 receptor proteins” and “DR3 polypeptides” refer to all DR3 receptor polypeptides or fragments or variants thereof as well as proteins resulting from the alternate splicing of the genomic DNA sequences encoding proteins having regions of amino acid sequence identity and receptor activity which correspond to the protein shown in SEQ ID NO:2 as well as DR3 allellic variants.

### Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the heavy and the light chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J Immunol. 148:1547 1553 (1992). In addition, bispecific antibodies may be formed as “diabodies” (Holliger et al. “‘Diabodies’: small bivalent and bispecific antibody fragments” PNAS USA 90:6444-6448 (1993)) or “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int. J. Cancer Suppl. 7:51-52 (1992)).

Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

### Anti-DR3 Receptor Antibodies

The present invention is directed to humanized antibodies, generally isolated, that specifically bind one or more DR3 receptor polypeptides. A panel of humanized heavy and light chain variable domains was produced by applying string content optimization (see, e.g., U.S. Pat. No. 7,657,380, issued Feb. 2, 2010, and U.S. Publ. No. 2008-0167449, filed Oct. 31, 2007). This is a method of humanization based on a metric of antibody humanness, termed human string content (HSC), which quantifies the humanness of a sequence by comparison to the human antibody germline at the level of potential MHC/T-cell epitopes. Two different scores are generated which are optimized by making amino acid substitutions in the murine antibody using residues from structurally analgous positions within the human antibody germline repertoire. These techniques were utilized in accordance with the present invention for the preparation of antibodies specific to DR3 receptor polypeptides. The production of a hybridoma cell line that produces antibodies specific to DR3 receptor polypeptides is described herein. Further, the antibodies, fragments, and derivatives thereof produced by this cell line are characterized.

Preferred antibodies of the invention include antibodies expressed by the 11H08 cell line and the antibodies expressed by any subclones of this line or fragments or variants thereof, including humanized variants.

The present invention encompasses antibodies (including molecules comprising, or alternatively consisting of, antibody fragments or variants thereof) that specifically bind to a DR3 receptor polypeptide or a fragment, variant, or fusion protein thereof. A DR3 receptor polypeptide includes, but is not limited to, DR3 (SEQ ID NO:2). Antibodies of the invention may specifically bind DR3 receptors including, but not limited to, DR3 as well as fragments and variants thereof, and are described in more detail below.

### DR3 Receptor Polypeptides

The antibodies of the present invention specifically bind DR3 polypeptidefragments, variants (including species orthologs and allelic variants of DR3), multimers or modified forms thereof. For example, an antibody specific for DR3 may bind the DR3 moiety of a fusion protein comprising all or a portion of DR3.

DR3 proteins may be found as monomers or multimers (i.e., dimers, trimers, tetramers, and higher multimers). Certain members of the TNF family of proteins are believed to exist in trimeric form (Beutler and Huffel, Science 264:667, 1994; Banner et al., Cell 73:431, 1993). Thus, trimeric DR3 may offer the advantage of enhanced biological activity. Accordingly, the present invention relates to antibodies that bind DR3 proteins found as monomers or as part of multimers. In specific embodiments, antibodies of the invention bind DR3 monomers, dimers, trimers or tetramers. In additional embodiments, antibodies of the invention bind at least dimers, at least trimers, or at least tetramers containing one or more DR3 polypeptides.

Antibodies of the invention may bind DR3 homomers or heteromers. As used herein, the term homomer, refers to a multimer containing only DR3 proteins of the invention (including DR3 fragments, variants, and fusion proteins, as described herein). These homomers may contain DR3 proteins having identical or different polypeptide sequences. In a specific embodiment, a homomer of the invention is a multimer containing only DR3 proteins having an identical polypeptide sequence. In another specific embodiment, antibodies of the invention bind DR3 homomers containing DR3 proteins having different polypeptide sequences. In specific embodiments, antibodies of the invention bind a DR3 homodimer (e.g., containing DR3 proteins having identical or different polypeptide sequences) or a homotrimer (e.g., containing DR3 proteins having identical or different polypeptide sequences). In additional embodiments, antibodies of the invention bind at least a homodimer, at least a homotrimer, or at least a homotetramer of DR3.

As used herein, the term heteromer refers to a multimer containing heterologous proteins (i.e., proteins containing polypeptide sequences that do not correspond to a polypeptide sequences encoded by the DR3 gene) in addition to the DR3 proteins of the invention. In a specific embodiment, antibodies of the invention bind a heterodimer, a heterotrimer, or a heterotetramer. In additional embodiments, the antibodies of the invention bind at least a homodimer, at least a homotrimer, or at least a homotetramer containing one or more DR3 polypeptides.

Multimers bound by one or more antibodies of the invention may be the result of hydrophobic, hydrophilic, ionic and/or covalent associations and/or may be indirectly linked, by for example, liposome formation. Thus, in one embodiment, multimers bound by one or more antibodies of the invention, such as, for example, homodimers or homotrimers, are formed when DR3 proteins contact one another in solution. In another embodiment, heteromultimers bound by one or more antibodies of the invention, such as, for example, heterotrimers or heterotetramers, are formed when proteins of the invention contact antibodies to the DR3 polypeptides (including antibodies to the heterologous polypeptide sequence in a fusion protein) in solution. In other embodiments, multimers bound by one or more antibodies of the invention are formed by covalent associations with and/or between the DR3 proteins of the invention. Such covalent associations may involve one or more amino acid residues contained in the polypeptide sequence of the protein (e.g., the polypeptide sequence recited in SEQ ID NO:2. In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a DR3 fusion protein. In one example, covalent associations are between the heterologous sequence contained in a fusion protein (see, e.g., U.S. Pat. No. 5,478,925). In a specific example, the covalent associations are between the heterologous sequence contained in a DR3-Fc fusion protein (as described herein). In another specific example, covalent associations of fusion proteins are between heterologous polypeptide sequences from another TNF family ligand/receptor member that is capable of forming covalently associated multimers, such as for example, oseteoprotegerin (see, e.g., International Publication No. WO 98/49305, the contents of which are herein incorporated by reference in its entirety).

Antibodies that bind DR3 receptor polypeptides may bind them as isolated polypeptides or in their naturally occurring state. For, example antibodies of the present invention may bind recombinantly produced DR3 receptor polypeptides. In a specific embodiment, antibodies of the present invention bind a DR3 receptor polypeptide expressed on the surface of a cell wherein the cell comprises a polynucleotide encoding SEQ ID NO:1 operably associated with a regulatory sequence that controls gene expression.

Antibodies of the present invention may bind DR3 polypeptide fragments comprising or alternatively, consisting of, an amino acid sequence contained in SEQ ID NO:2, or encoded by nucleic acids which hybridize (e.g., under stringent hybridization conditions) to the nucleotide sequence or the complementary strand thereto. Protein fragments may be “free-standing,” or comprised within a larger polypeptide of which the fragment forms a part or region, most preferably as a single continuous region. Antibodies of the present invention may bind polypeptide fragments, including, for example, fragments that comprise or alternatively, consist of from about amino acid residues: 1 to 11, 22 to 45, 48 to 71, 85 to 101, 111 to 122, 150 to 166, 168 to 179, and/or 185 to 194 of SEQ ID NO:2. In this context “about” includes the particularly recited value, larger or smaller by several (5, 4, 3, 2, or 1) amino acids, at either extreme or at both extremes. Moreover, polypeptide fragments bound by the antibodies of the invention can be at least about 10, 20, 30, 40, 50, 60, 70, 80, 90, 100, 110, 120, 130, 140, 150, 175 or 200 amino acids in length. In this context “about” includes the particularly recited value, larger or smaller by several (5, 4, 3, 2, or 1) amino acids, at either extreme or at both extremes.

Preferably, antibodies of the present invention bind polypeptide fragments selected from the group: a polypeptide comprising or alternatively, consisting of, the DR3 receptor ligand binding domain (predicted to constitute amino acid residues from about 25 to about 201 in SEQ ID NO:2); a polypeptide comprising or alternatively, consisting of, a DR3 cysteine rich domains (which may be found in the protein fragment consisting of amino acid residues from about 47 to about 195 in SEQ ID NO:2); a polypeptide comprising or alternatively, consisting of, the DR3 receptor transmembrane domain (predicted to constitute amino acid residues from about 202 to about 224 in SEQ ID NO:2); a polypeptide comprising or alternatively, consisting of, the full length polypeptide (amino acid residues 1 to 417); a polypeptide comprising or alternatively, consisting of, fragment of the predicted mature DR3 polypeptide, wherein the fragment has a DR3 functional activity (e.g., antigenic activity or biological acitivity); a polypeptide comprising or alternatively, consisting of, the DR3 receptor intracellular domain (predicted to constitute amino acid residues from about 225 to about 417 in SEQ ID NO:2); and a polypeptide comprising, or alternatively, consisting of, one, two, three, four or more, epitope bearing portions of the DR3 receptor protein. In additional embodiments, the polypeptide fragments of the invention comprise, or alternatively, consist of, any combination of 1, 2, 3, 4, or all 5 of the above members. The amino acid residues constituting the DR3 receptor extracellular, transmembrane and intracellular domains have been predicted by computer analysis. Thus, as one of ordinary skill would appreciate, the amino acid residues constituting these domains may vary slightly (e.g., by about 1 to about 15 amino acid residues) depending on the criteria used to define each domain. Polynucleotides encoding these polypeptides are also encompassed by the invention.

It is believed that one of the extracellular cysteine rich motifs of DR3 is important for interactions between DR3 and DR3 ligand (e.g. TL1A). Accordingly, in highly preferred embodiments, antibodies of the present invention bind DR3 polypeptide fragments comprising, or alternatively consisting of amino acid residues from about 25 to about 201 in SEQ ID NO:2. In another preferred embodiment, antibodies of the present invention bind DR3 polypeptides comprising, or alternatively consisting the extracellular soluble domain of DR3 (amino acid residues 25-201 of SEQ ID NO:2) In highly preferred embodiments, the antibodies of the invention that bind all or a portion of the extracellular soluble domain of DR3 prevent DR3 ligand (e.g. TL1A) from binding to DR3. In other highly preferred embodiments, the antibodies of the invention that bind all or a portion of the extracellular soluble domain of DR3 antagonize the DR3 receptor. In other highly preferred embodiments, the antibodies of the invention that bind all or a portion of the extracellular soluble domain of DR3 inhibit proliferation of the cells expressing the DR3 receptor. In other highly preferred embodiments, the antibodies of the invention that bind all or a portion of the extracellular soluble domain of DR3 inhibit differentiation of the cells expressing the DR3 receptor (e.g. T-cells).

Antibodies of the invention may also bind fragments comprising, or alternatively, consisting of structural or functional attributes of DR3. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of complete (i.e., full-length) DR3. Certain preferred regions include, but are not limited to, regions of the aforementioned types identified by analysis of the amino acid sequence depicted in (SEQ ID NO:2), such preferred regions include; Garnier-Robson predicted alpha-regions, beta-regions, turn-regions, and coil-regions; Chou-Fasman predicted alpha-regions, beta-regions, and turn-regions; Kyte-Doolittle predicted hydrophilic regions; Eisenberg alpha and beta amphipathic regions; Emini surface-forming regions; and Jameson-Wolf high antigenic index regions, as predicted using the default parameters of these computer programs.

In another aspect, the invention provides an antibody that binds a peptide or polypeptide comprising an epitope-bearing portion of a polypeptide described herein. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983) Antibodies that react with predetermined sites on proteins. Science 2/9:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides are therefore useful to raise antibodies, including monoclonal antibodies, fragments or variants thereof, that bind to a DR3 polypeptide of the invention. See, for instance, Wilson et al., Cell 37:767-778 (1984) at 777. Antigenic epitope-bearing peptides and polypeptides preferably contain a sequence of at least seven, more preferably at least nine and most preferably between at least about 15 to about 30 amino acids contained within the amino acid sequence of SEQ ID NO:2.

Antibodies, fragments, or variants thereof of the invention may bind one or more antigenic DR3 polypeptides or peptides including, but not limited to: a polypeptide comprising amino acid residues from about 1 to about 11 of SEQ ID NO:2; a polypeptide comprising amino acid residues from about 22 to about 45 of SEQ ID NO:2; a polypeptide comprising amino acid residues from about 148 to about 71 of SEQ ID NO:2; a polypeptide comprising amino acid residues from about 85 to about 101 of SEQ ID NO:2; a polypeptide comprising amino acid residues from about 111 to about 122 of SEQ ID NO:2; a polypeptide comprising amino acid residues from about 150 to about 166 of SEQ ID NO:2; a polypeptide comprising amino acid residues from about 168 to about 179 of SEQ ID NO:2; and/or a polypeptide comprising amino acid residues from about 185 to about 194 of SEQ ID NO:2. In this context “about” includes the particularly recited range, larger or smaller by several (5, 4, 3, 2, or 1) amino acids, at either terminus or at both termini. As indicated above, the inventors have determined that the above polypeptide fragments are antigenic regions of the DR3 protein. Epitope-bearing DR3 peptides and polypeptides may be produced by any conventional means. Houghten, R. A., “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, DR3 polypeptides and the epitope-bearing fragments thereof described herein (e.g., corresponding to all or a portion of the extracellular domain such as, for example, amino acid residues 1 to 201 of SEQ ID NO:2 can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric DR3 protein or protein fragment alone (Fountoulakis et al., J Biochem 270:3958-3964 (1995)). Thus, antibodies of the invention may bind the DR3 moiety of fusion proteins that comprise all or a portion of a DR3 receptor polypeptide.

Thus, antibodies of the present invention may bind a fragment, derivative, allelic variant, or analog of the polypeptide of SEQ ID NO:2. Such fragments, variants or derivatives may be (i) one in which at least one or more of the amino acid residues are substituted with a conserved or non-conserved amino acid residue (preferably a conserved amino acid residue(s), and more preferably at least one but less than ten conserved amino acid residues) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

In specific embodiments, the number of substitutions, additions or deletions in the amino acid sequence of SEQ ID NO:2 and/or any of the polypeptide fragments described herein (e.g., the extracellular domain or intracellular domain) is 75, 70, 60, 50, 40, 35, 30, 25, 20, 15, 10, 9, 8, 7, 6, 5, 4, 3, 2, 1 or 30-20, 20-15, 20-10, 15-10, 10-1, 5-10, 1-5, 1-3 or 1-2.

In preferred embodiments, antibodies of the present invention bind regions of DR3 that are essential for DR3 function. In other preferred embodiments, antibodies of the present invention bind regions of DR3 that are essential for DR3 function and inhibit or abolish DR3 function. In other preferred embodiments, antibodies of the present invention bind regions of DR3 that are essential for DR3 function and enhance DR3 function.

Thus, the invention also encompasses antibodies that bind DR3 derivatives and analogs that have one or more amino acid residues deleted, added, or substituted to generate DR3 polypeptides that are better suited for expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognition sequences in the DR3 polypeptides and/or an amino acid deletion at the second position of any one or more such recognition sequences will prevent glycosylation of the DR3 at the modified tripeptide sequence (see, e.g., Miyajimo et al., EMBO J 5(6):1193-1197). Additionally, one or more of the amino acid residues of DR3 polypeptides (e.g., arginine and lysine residues) may be deleted or substituted with another residue to eliminate undesired processing by proteases such as, for example, furins or kexins.

### Antibodies of the Invention May Bind Modified DR3 Receptor Polypeptides

It is specifically contemplated that antibodies, fragments or variants thereof, of the present invention may bind modified forms of DR3 receptor proteins (e.g., SEQ ID NO:2).

In specific embodiments, antibodies, fragments or variants thereof, of the present invention bind DR3 receptor polypeptides (such as those described above) including, but not limited to naturally purified DR3 receptor polypeptides, DR3 receptor polypeptides produced by chemical synthetic procedures, and DR3 receptor polypeptides produced by recombinant techniques from a prokaryotic or eukaryotic host, including, for example, bacterial, yeast, higher plant, insect and mammalian cells using, for example, the recombinant compositions and methods described above. Depending upon the host employed in a recombinant production procedure, the polypeptides may be glycosylated or non-glycosylated. In addition, DR3 receptor polypeptides may also include an initial modified methionine residue, in some cases as a result of host-mediated processes.

The invention additionally encompasses antibodies that bind DR3 receptor polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin; etc.

Additional post-translational modifications to DR3 receptor polypeptides for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are antibodies that bind chemically modified derivatives of DR3 receptor polypeptide which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

As mentioned, the antibodies of the present invention may bind DR3 receptor polypeptides that are modified by either natural processes, such as posttranslational processing, or by chemical modification techniques which are well known in the art. It will be appreciated that the same type of modification may be present in the same or varying degrees at several sites in a given DR3 receptor polypeptide. DR3 receptor polypeptides may be branched, for example, as a result of ubiquitination, and they may be cyclic, with or without branching. Cyclic, branched, and branched cyclic DR3 receptor polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

### Anti-DR3 Receptor Antibodies

In one embodiment, the invention provides antibodies, fragments, or variants thereof that specifically bind a DR3 receptor polypeptide (e.g., SEQ ID NO:2) or fragments or variants thereof, wherein the amino acid sequence of the heavy chain and the amino acid sequence of the light chain are the same as, or variants of, the amino acid sequence of a heavy chain and a light chain expressed by the hybridoma cell line 11H08. The sequences of the VH and VL domains of the invention are listed in Table 1. Specific binding to DR3 receptor polypeptides may be determined by immunoassays known in the art or described herein for assaying specific antibody-antigen binding. Molecules comprising, or alternatively consisting of, fragments or variants of these antibodies that specifically bind to a DR3 receptor are also encompassed by the invention, as are nucleic acid molecules encoding these antibodies, molecules, fragments and/or variants.

TABLE 1

| | | SEQ ID | AAs of | AAs of | AAs of |
|---|---|---|---|---|---|
| Domain | Sequence Name | NO: | CDR1 | CDR2 | CDR3 |
| VH | 11H08_H0 | 7 | 31-35 | 50-66 | 99-110 |
| | 11H08_H0.1 | 8 | 31-35 | 50-66 | 99-110 |
| | 11H08_H1 | 9 | 31-35 | 50-66 | 99-110 |
| | 11H08_H1.1 | 10 | 31-35 | 50-66 | 99-110 |
| | 11H08_H2 | 11 | 31-35 | 50-66 | 99-110 |
| | 11H08_H2.1 | 12 | 31-35 | 50-66 | 99-110 |
| | 11H08_H3 | 13 | 31-35 | 50-66 | 99-110 |
| | 11H08_H3.1 | 14 | 31-35 | 50-66 | 99-110 |
| | 11H08_H4 | 15 | 31-35 | 50-66 | 99-110 |
| | 11H08_H4.2 | 16 | 31-35 | 50-66 | 99-110 |
| | 11H08_H5 | 17 | 31-35 | 50-66 | 99-110 |
| | 11H08_H5.2 | 18 | 31-35 | 50-66 | 99-110 |
| | 11H08_H5.3 | 19 | 31-35 | 50-66 | 99-110 |
| VL | 11H08_L0 | 20 | 24-38 | 54-60 | 93-101 |
| | 11H08_L1 | 21 | 24-38 | 54-60 | 93-101 |
| | 11H08_L2 | 22 | 24-38 | 54-60 | 93-101 |
| | 11H08_L3 | 23 | 24-38 | 54-60 | 93-101 |
| | 11H08_L4 | 24 | 24-38 | 54-60 | 93-101 |

Representative clones of the above sequences were deposited with the American Type Culture Collection (herein referred to as “ATCC®”). PTA-11939 comprising the cDNA encoding VL 11H08_L2 (SEQ ID NO:22) and PTA-11940 comprising the cDNA encoding VH 11H08_H1.1 (SEQ ID NO:10) were deposited with the ATCC® on Jun. 14, 2011. The ATCC® is located at 10801 University Boulevard, Manassas, Va. 20110-2209, USA. The ATCC® deposits were made pursuant to the terms of the Budapest Treaty on the international recognition of the deposit of microorganisms for the purposes of patent procedure.

In one embodiment of the present invention, antibodies, fragments, or variants thereof that specifically bind to a DR3 receptor or a fragment or variant thereof, comprise a polypeptide having the amino acid sequence of any one of the heavy chains referred to in Table 1 and/or any one of the light chains referred to in Table 1.

The present invention also provides antibodies that specifically bind to a polypeptide, or polypeptide fragment or variant of a DR3 receptor, wherein said antibodies comprise, or alternatively consist of, a polypeptide having an amino acid sequence of any one, two, or three of the VH CDRs contained in one or more of the VH domains referred to in Table 1. In particular, the invention provides antibodies that specifically bind a DR3 receptor, comprising, or alternatively consisting of, a polypeptide having the amino acid sequence of a VH CDR1 contained in one or more of the VH domains referred to in Table 1. In another embodiment, antibodies that specifically bind a DR3 receptor, comprise, or alternatively consist of, a polypeptide having the amino acid sequence of a VH CDR2 contained in one or more of the VH domains referred to in Table 1. In a preferred embodiment, antibodies that specifically bind a DR3 receptor, comprise, or alternatively consist of a polypeptide having the amino acid sequence of a VH CDR3 contained in one or more of the VH domains referred to in Table 1. Molecules comprising, or alternatively consisting of, these antibodies, or antibody fragments or variants thereof, that specifically bind to DR3 receptor or a DR3 receptor fragment or variant thereof are also encompassed by the invention, as are nucleic acid molecules encoding these antibodies, molecules, fragments and/or variants.

The present invention also provides antibodies that specifically bind to a polypeptide, or polypeptide fragment or variant of a DR3 receptor, wherein said antibodies comprise, or alternatively consist of, a polypeptide having an amino acid sequence of any one, two, or three of the VL CDRs contained in one or more of the VL domains referred to in Table 1. In particular, the invention provides antibodies that specifically bind a DR3 receptor, comprising, or alternatively consisting of, a polypeptide having the amino acid sequence of a VL CDR1 contained in one or more of the VL domains referred to in Table 1. In another embodiment, antibodies that specifically bind a DR3 receptor, comprise, or alternatively consist of, a polypeptide having the amino acid sequence of a VL CDR2 contained in one or more of the VL domains referred to in Table 1. In a preferred embodiment, antibodies that specifically bind a DR3 receptor, comprise, or alternatively consist of a polypeptide having the amino acid sequence of a VL CDR3 contained in one or more of the VL domains referred to in Table 1. Molecules comprising, or alternatively consisting of, these antibodies, or antibody fragments or variants thereof, that specifically bind to DR3 receptor or a DR3 receptor fragment or variant thereof are also encompassed by the invention, as are nucleic acid molecules encoding these antibodies, molecules, fragments and/or variants.

The present invention also provides antibodies (including molecules comprising, or alternatively consisting of, antibody fragments or variants) that specifically bind to a DR3 receptor polypeptide or polypeptide fragment or variant of a DR3 receptor, wherein said antibodies comprise, or alternatively consist of, one, two, or three VH CDRs and one, two, or three VL CDRs, as contained in one or more of the VH domains or VL domains referred to in Table 1. In particular, the invention provides for antibodies that specifically bind to a polypeptide or polypeptide fragment or variant of a DR3 receptor, wherein said antibodies comprise, or alternatively consist of, a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof, of the VH CDRs and VL CDRs contained in one or more of the VH domains or VL domains referred to in Table 1. Molecules comprising, or alternatively consisting of, fragments or variants of these antibodies, that specifically bind to DR3 receptor are also encompassed by the invention, as are nucleic acid molecules encoding these antibodies, molecules, fragments or variants.

### Nucleic Acid Molecules Encoding Anti-DR3 Receptor Antibodies

The present invention also provides for nucleic acid molecules, generally isolated, encoding an antibody of the invention (including molecules comprising, or alternatively consisting of, antibody fragments or variants thereof). In specific embodiments, the nucleic acid molecules comprise, or alternatively consist of nucleic acid molecuels encoding an antibody of the invention or fragments or variants thereof.

In a specific embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains referred to in Table 1 and a VL domain having an amino acid sequence of any one of the VL domains referred to in Table 1. In another embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains referred to in Table 1 or a VL domain having an amino acid sequence of any one of the VL domains referred to in Table 1.

The present invention also provides antibodies that comprise, or alternatively consist of, variants (including derivatives) of the antibody molecules (e.g., the VH domains and/or VL domains) described herein, which antibodies specifically bind to a DR3 receptor or fragment or variant thereof. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain, VHCDR1, VHCDR2, VHCDR3, VL domain, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a DR3 receptor).

The polynucleotide variants of the invention may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, polypeptide variants in which less than 50, less than 40, less than 30, less than 20, less than 10, or 5-50, 5-25, 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host, such as, yeast or E. coli).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybriodma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g, improvements in antigen binding activity or change in antibody specificity). Following mutagenesis, the encoded protein may routinely be expressed and the functional and/or biological activity of the encoded protein, (e.g., ability to specifically bind a DR3 receptor) can be determined using techniques described herein or by routinely modifying techniques known in the art.

In a specific embodiment, an antibody of the invention (including a molecule comprising, or alternatively consisting of, an antibody fragment or variant thereof), that specifically binds DR3 receptor polypeptides or fragments or variants thereof, comprises, or alternatively consists of, an amino acid sequence encoded by a nucleotide sequence that hybridizes to a nucleotide sequence that is complementary to that encoding at least one of the VH or VL domains referred to in Table 1. under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3). Nucleic acid molecules encoding these antibodies are also encompassed by the invention.

It is well known within the art that polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that specifically binds to a DR3 receptor polypeptide or fragments or variants of a DR3 receptor polypeptide, comprises, or alternatively consists of, a VH domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of at least one of the VH domains referred to in Table 1.

In another embodiment, an antibody (including a molecule comprising, or alternatively consisting of, an antibody fragment or variant thereof), that specifically binds to a DR3 receptor polypeptide or fragments or variants of a DR3 receptor polypeptide, comprises, or alternatively consists of, a VL domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of at least one of the VL domains referred to in Table 1.

### Methods of Producing Antibodies

Monoclonal antibodies specific for DR3 receptor polypeptides were prepared using hybridoma technology. Typical procedures for generating a mouse monoclonal antibody are well-known in the art and are described by Kohler and Millstein (1975) and others. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981)). Briefly, mice are immunized with DR3-Fc. After immunization, the splenocytes of such mice are extracted and fused with a suitable myeloma cell line. Any suitable myeloma cell line may be employed in accordance with the present invention. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the DR3 receptor polypeptides.

In one embodiment, the present invention provides hybridoma cell lines expressing an antibody of the invention. In a preferred embodiment, the hybridoma cell line is 11H08. In another preferred embodiment, the invention provides humanized variants of the hybridoma 11H08. In another preferred embodiment, the invention provides humanized variants of the hybridoma 11H08 fused to a heterologous polypeptide.

### Additional Methods of Producing Antibodies

Antibodies in accordance with the invention may also be prepared using a phage scFv display library. Technologies utilized for achieving the same are disclosed in the patents, applications, and references disclosed herein.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH and VL domains are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest (i.e., a DR3 receptor polypeptide or a fragment thereof) can be selected or identified with antigen, e.g., using labeled antigen or antigen bound or captured to a solid surface or bead. Examples of phage display methods that can be used to make the antibodies of the present invention include, but are not limited to, those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/O1 134; PCT publications WO 90/02809; WO 91/10737; WO 92/01047; WO 92/18719; WO 93/1 1236; WO 95/15982; WO 95/20401; WO97/13844; and U.S. Pat. Nos. 5,698,426; 5,223,409; 5,403,484; 5,580,717; 5,427,908; 5,750,753; 5,821,047; 5,571,698; 5,427,908; 5,516,717; 5,780,225; 5,658,727; 5,735,743 and 5,969,108; each of which is incorporated herein by reference in its entirety.

For some uses, such as for in vitro affinity maturation of an antibody of the invention, it may be useful to express one or more of the VH and VL domains referred to in Table 1 as single chain antibodies or Fab fragments in a phage display library. For example, the cDNAs encoding the VH and VL domains referred to in Table 1 may be expressed in all possible combinations using a phage display library, allowing for the selection of VH/VL combinations that bind a DR3 receptor polypeptides with preferred binding characteristics such as improved affinity or improved off rates. Additionally, VH and VL segments—the CDR regions of the VH and VL domains \referred to in Table 1, in particular, may be mutated in vitro. Expression of VH and VL domains with “mutant” CDRs in a phage display library allows for the selection of VH/VL combinations that bind a DR3 receptor polypeptides with preferred binding characteristics such as improved affinity or improved off rates. Antibodies of the invention (including antibody fragments or variants) can be produced by any method known in the art. For example, it will be appreciated that antibodies in accordance with the present invention can be expressed in cell lines other than hybridoma cell lines. Sequences encoding the cDNAs or genomic clones for the particular antibodies can be used for transformation of a suitable mammalian or nonmammalian host cells or to generate phage display libraries, for example. Additionally, polypeptide antibodies of the invention may be chemically synthesized or produced through the use of recombinant expression systems.

One way to produce the antibodies of the invention would be to clone the VH and/or VL domains referred to in Table 1. In order to isolate the VH and VL domains from hybridoma cell lines, PCR primers complementary to VH or VL nucleotide sequences may be used to amplify the VH and VL sequences contained in total RNA isolated from hybridoma cell lines. The PCR products may then be cloned using vectors, for example, which have a PCR product cloning site consisting of a 5′ and 3′ single T nucleotide overhang, that is complementary to the overhanging single adenine nucleotide added onto the 5′ and 3′ end of PCR products by many DNA polymerases used for PCR reactions. The VH and VL domains can then be sequenced using conventional methods known in the art.

The cloned VH and VL genes may be placed into one or more suitable expression vectors. By way of non-limiting example, PCR primers including VH or VL nucleotide sequences, a restriction site, and a flanking sequence to protect the restriction site may be used to amplify the VH or VL sequences. Utilizing cloning techniques known to those of skill in the art, the PCR amplified VH domains may be cloned into vectors expressing the appropriate immunoglobulin constant region, e.g., the human IgG1 or IgG4 constant region for VH domains, and the human kappa or lambda constant regions for kappa and lambda VL domains, respectively. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into a single vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art (See, for example, Guo et al., J. Clin. Endocrinol. Metab. 82:925-31 (1997), and Ames et al., J. Immunol. Methods 184:177-86 (1995) which are herein incorporated in their entireties by reference).

The invention provides polynucleotides comprising, or alternatively consisting of, a nucleotide sequence encoding an antibody of the invention (including molecules comprising, or alternatively consisting of, antibody fragments or variants thereof). The invention also encompasses polynucleotides that hybridize under high stringency, or alternatively, under intermediate or lower stringency hybridization conditions, e.g., as defined supra, to polynucleotides complementary to nucleic acids having a polynucleotide sequence that encodes an antibody of the invention or a fragment or variant thereof.

The polynucleotides may be obtained, and the nucleotide sequence of the polynucleotides determined, by any method known in the art. If the amino acid sequences of the VH domains, VL domains and CDRs thereof, are known, nucleotide sequences encoding these antibodies can be determined using methods well known in the art, i.e., the nucleotide codons known to encode the particular amino acids are assembled in such a way to generate a nucleic acid that encodes the antibody, of the invention. Such a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody (including molecules comprising, or alternatively consisting of, antibody fragments or variants thereof) may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells or Epstein Barr virus transformed B cell lines that express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody (including molecules comprising, or alternatively consisting of, antibody fragments or variants thereof) is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the VH and VL domains of heavy and light chains referred to in Table 1, or fragments or variants thereof, are inserted within antibody framework regions using recombinant DNA techniques known in the art. In a specific embodiment, one, two, three, four, five, six, or more of the CDRs of the heavy and light chains referred to in Table 1, or fragments or variants thereof, is inserted within antibody framework regions using recombinant DNA techniques known in the art. The framework regions may be naturally occurring or consensus antibody framework regions, and preferably human antibody framework regions (see, e.g., Chothia et al., J. Mol. Biol. 278: 457-479 (1998) for a listing of human antibody framework regions, the contents of which are hereby incorporated by reference in its entirety). Preferably, the polynucleotides generated by the combination of the antibody framework regions and CDRs encode an antibody (including molecules comprising, or alternatively consisting of, antibody fragments or variants thereof) that specifically binds to a DR3 receptor. Preferably, as discussed supra, polynucleotides encoding variants of antibodies or antibody fragments having one or more amino acid substitutions may be made within the framework regions, and, preferably, the amino acid substitutions do not significantly alter binding of the antibody to its antigen. Additionally, such methods may be used to make amino acid substitutions or deletions of one or more variable region cysteine residues participating in an intrachain disulfide bond to generate antibody molecules, or antibody fragments or variants, lacking one or more intrachain disulfide bonds. Other alterations to the polynucleotide are encompassed by the present invention and fall within the ordinary skill of the art.

A chimeric antibody is a molecule in which different portions of the antibody are derived from different immunoglobulin molecules such as antibodies having a variable region derived from a human antibody and a non-human (e.g., murine) immunoglobulin constant region or vice versa. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entirety. Chimeric antibodies comprising one or more CDRs from human species and framework regions from a non-human immunoglobulin molecule (e.g., framework regions from a murine, canine or feline immunoglobulin molecule) (or vice versa) can be produced using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352). In a preferred embodiment, chimeric antibodies comprise a human CDR3 having an amino acid sequence of any one of the VH CDR3s or VL CDR3s of a VH or VL domain of a heavy or light chain referred to in Table 1, or a variant thereof. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 352:323 (1988), which are incorporated herein by reference in their entireties.)

Intrabodies are antibodies, often scFvs, that are expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies of the invention can be produced using methods known in the art, such as those disclosed and reviewed in Chen et al., Hum. Gene Ther. 5:595-601 (1994); Marasco, W. A., Gene Ther. 4:11-15 (1997); Rondon and Marasco, Annu. Rev. Microbiol. 51:257-283 (1997); Proba et al., J. Mol. Biol. 275:245-253 (1998); Cohen et al., Oncogene 17:2445-2456 (1998); Ohage and Steipe, J. Mol. Biol. 291:1119-1128 (1999); Ohage et al., J. Mol. Biol. 291:1129-1134 (1999); Wirtz and Steipe, Protein Sci. 8:2245-2250 (1999); Zhu et al., J. Immunol. Methods 231:207-222 (1999); and references cited therein.

Recombinant expression of an antibody of the invention (including antibody fragments or variants thereof (e.g., a heavy or light chain of an antibody of the invention), requires construction of an expression vector(s) containing a polynucleotide that encodes the antibody. Once a polynucleotide encoding an antibody molecule (e.g., a whole antibody, a heavy or light chain of an antibody, or portion thereof (preferably, but not necessarily, containing the heavy or light chain variable domain)), of the invention has been obtained, the vector(s) for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention (e.g., a whole antibody, a heavy or light chain of an antibody, a heavy or light chain variable domain of an antibody, or a portion thereof, or a heavy or light chain CDR, a single chain Fv, or fragments or variants thereof), operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464, the contents of each of which are hereby incorporated by reference in its entirety) and the variable domain of the antibody may be cloned into such a vector for expression of the entire heavy chain, the entire light chain, or both the entire heavy and light chains.

The expression vector(s) is(are) transferred to a host cell by conventional techniques and the transfected cells are then cultured by conventional techniques to produce an antibody of the invention. Thus, the invention includes host cells containing polynucleotide(s) encoding an antibody of the invention (e.g., whole antibody, a heavy or light chain thereof, or portion thereof, or a single chain antibody, or a fragment or variant thereof), operably linked to a heterologous promoter. In a preferred embodiment, for the expression of antibody fragments, vectors encoding both the heavy and light chains may be co-expressed in the host cell for expression of the antibody fragment. In another preferred embodiment, for the expression of entire antibody molecules, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include, but are not limited to, bacteriophage particles engineered to express antibody fragments or variants thereof (single chain antibodies), microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3, NS0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990); Bebbington et al., Bio/Techniques 10:169 (1992); Keen and Hale, Cytotechnology 18:207 (1996)). These references are incorporated in their entirities by reference herein.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., EMBO 1. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) may be used as a vector to express foreign genes. The virus grows in Spodoptera frugiperda cells. Antibody coding sequences may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, Proc. Natl. Acad. Sci. USA 8 1:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, NSO, MDCK, 293, 3T3, W138, and in particular, breast cancer cell lines such as, for example, BT483, Hs578T, HTB2, BT2O and T47D, and normal mammary gland cell line such as, for example, CRL7O3O and HsS78Bst.

For long-term, high-yield production of recombinant proteins, stable expression is preferred. For example, cell lines which stably express the antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:8 17 (1980)) genes can be employed in tk−, hgprt− or aprt− cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62: 191-217 (1993); TIB TECH 11(5):155-2 15 (May, 1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, “The use of vectors based on gene amplification for the expression of cloned genes in mammalian cells” in DNA Cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the coding sequence of the antibody, production of the antibody will also increase (Crouse et al., Mol. Cell. Biol. 3:257 (1983)).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NS0) which are glutamine synthase negative. Glutamine synthase expression systems can also function in glutamine synthase expressing cells (e.g. Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/01036; WO89/10404; and WO91/06657 which are incorporated in their entireties by reference herein. Additionally, glutamine synthase expression vectors that may be used according to the present invention are commercially available from suppliers, including, for example Lonza Biologics, Inc. (Portsmouth, N.H.). Expression and production of monoclonal antibodies using a GS expression system in murine myeloma cells is described in Bebbington et al., Bio/technology 10:169 (1992) and in Biblia and Robinson Biotechnol. Prog. 11:1 (1995) which are incorporated in their entirities by reference herein.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain is preferably placed before the heavy chain to avoid an excess of toxic free heavy chain (Proudfoot, Nature 322:52 (1986); Kohler, Proc. Natl. Acad. Sci. USA 77:2 197 (1980)). The coding sequences for the heavy and light chains may comprise cDNA or genomic DNA.

Once an antibody molecule of the invention (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, such as, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art, to facilitate purification.

While searching for antibodies that antagonize DR3 activity, whole immunoglobulin antibody molecules were tested. Of the antibodies tested that bound DR3 and blocked TL1A ligand binding to the DR3 receptor, all proved to be agonistic and resulted in activation of and consequent signaling through DR3. In an effort to identify antagonist molecules, monomeric antibody molecules, including scFv and Fab molecules, were generated. Suprisingly, the scFv molecules tested also were found to have agonistic properties and activate DR3 signaling. However, unlike the whole immunoglobulin antibodies and the scFv molecules, Fab fragments of one of the anti-DR3 antibodies functioned antagonistically, demonstrating inhibition of the DR3 receptor.

Although whole immunoglobulin antibodies have a significant half-life in vivo after administration to an organism, the half-life of antibody fragments such as Fab fragments can be much shorter. It is known in the art that certain molecules can be added to a protein to extend their effective half-life in vivo (discussed in greater detail herein). To lengthen the half-life of the Fab molecules in vivo while still retaining antagonist activity, a portion of serum albumin protein was fused to the Fab fragment.

### Characterization of Anti-DR3 Receptor Antibodies

Antibodies of the present invention (including molecules comprising, or alternatively consisting of, antibody fragments or variants thereof) may also be described or specified in terms of their binding to DR3 receptor polypeptides or fragments or variants of DR3 receptor polypeptides. In specific embodiments, antibodies of the invention bind DR3 receptor polypeptides, or fragments or variants thereof, with a dissociation constant or K_{D} of less than or equal to 5×10⁻² M, 10⁻² M, 5×10⁻³ M, 10⁻³ M, 5×10⁻⁴ M, 10⁻⁴ M, 5×10⁻⁵ M, or 10⁻⁵ M. More preferably, antibodies of the invention bind DR3 receptor polypeptides or fragments or variants thereof with a dissociation constant or K_{D} less than or equal to 5×10⁻⁶ M, 10⁻⁶ M, 5×10⁻⁷ M, 10⁻⁷M, 5×10⁻⁸ M, or 10⁻⁸ M. Even more preferably, antibodies of the invention bind DR3 receptor polypeptides or fragments or variants thereof with a dissociation constant or K_{D} less than or equal to 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×⁻¹³ M, 10⁻¹³ M, 5×10⁻¹⁴ M, 10⁻¹⁴ M, 5×10⁻¹⁵ M, or 10⁻¹⁵ M. The invention encompasses antibodies that bind DR3 receptor polypeptides with a dissociation constant or K_{D} that is within any one of the ranges that are between each of the individual recited values.

In specific embodiments, antibodies of the invention bind DR3 receptor polypeptides or fragments or variants thereof with an off rate (k_{off}) of less than or equal to 5×10⁻² sec⁻¹, 10⁻² sec⁻¹, 5×10⁻³ sec⁻¹ or 10⁻³ sec⁻¹. More preferably, antibodies of the invention bind DR3 receptor polypeptides or fragments or variants thereof with an off rate (k_{off}) less than or equal to 5×10⁻⁴ sec⁻¹, 10⁻⁴ sec⁻¹, 5×10⁻⁵ sec⁻¹, or 10⁻⁵ sec⁻¹⁵×10⁻⁶ sec⁻¹, 10⁻⁶ sec⁻¹, 5×10⁻⁷ sec⁻¹ or 10⁻⁷ sec⁻¹. The invention encompasses antibodies that bind DR3 receptor polypeptides with an off rate (k_{off}) that is within any one of the ranges that are between each of the individual recited values.

In other embodiments, antibodies of the invention bind DR3 receptor polypeptides or fragments or variants thereof with an on rate (kₒₙ) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, antibodies of the invention bind DR3 receptor polypeptides or fragments or variants thereof with an on rate (kₒₙ) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷M⁻¹ sec⁻¹. The invention encompasses antibodies that bind DR3 receptor polypeptides with on rate (kₒₙ) that is within any one of the ranges that are between each of the individual recited values.

The antibodies of the invention (including molecules comprising, or alternatively consisting of, antibody fragments or variants thereof) specifically bind to a polypeptide or polypeptide fragment or variant of human DR3 receptor polypeptide (SEQ ID NO:2). In another embodiment, the antibodies of the invention specifically bind to a polypeptide or polypeptide fragment or variant of simian DR3 receptor polypeptides. In yet another embodiment, the antibodies of the invention specifically bind to a polypeptide or polypeptide fragment or variant of murine DR3 receptor polypeptides. In one embodiment, the antibodies of the invention bind specifically to human and simian DR3 receptor polypeptides. In another embodiment, the antibodies of the invention bind specifically to human DR3 receptor polypeptides and murine DR3 receptor polypeptides. More preferably, antibodies of the invention, preferentially bind to human DR3 receptor polypeptides compared to murine DR3 receptor polypeptides.

In preferred embodiments, the antibodies of the present invention (including molecules comprising, or alternatively consisting of, antibody fragments or variants thereof), specifically bind to DR3 receptor polypeptides and do not cross-react with any other antigens. In preferred embodiments, the antibodies of the invention specifically bind to DR3 receptor polypeptide (e.g., SEQ ID NO:2 or fragments or variants thereof) and do not cross-react with one or more additional members of the Tumor Necrosis Factor Receptor Family polypeptides (e.g., BCMA, TACI, CD30, CD27, OX40, 4-1BB, CD40, NGFR, TNFR1, TNFR2, Fas, and NGFR).

In another embodiment, the antibodies of the present invention (including molecules comprising, or alternatively consisting of, antibody fragments or variants thereof), specifically bind to DR3 receptor polypeptides and cross-react with other antigens. In other embodiments, the antibodies of the invention specifically bind to DR3 receptor polypeptide (e.g., SEQ ID NO:2 or fragments or variants thereof) and cross-react with one or more additional members of the Tumor Necrosis Factor Receptor Family polypeptides (e.g., BCMA, TACI, CD30, CD27, OX40, 4-1BB, CD40, NGFR, TNFR1, TNFR2, Fas, and NGFR).

The invention also encompasses antibodies (including molecules comprising, or alternatively consisting of, antibody fragments or variants thereof) that have one or more of the same biological characteristics as one or more of the antibodies described herein. By “biological characteristics” is meant, the in vitro or in vivo activities or properties of the antibodies, such as, for example, the ability to bind to DR3 receptor polypeptides (e.g., DR3 receptors expressed on the surface of a cell), the ability to inhibit DR3 receptor mediated biological activity (e.g., to inhibit proliferation and/or activation of DR3 receptor expressing cells); the ability to substantially block binding of a DR3 receptor ligand (e.g. TL1A), or a fragment, variant or fusion protein thereof, to DR3 receptor; or the ability to downregulate DR3 receptor expression on the surface of cells. Other biological activities that antibodies against DR3 receptor polypeptides may have, include, but are not limited to, the ability to stimulate DR3 receptor mediated biological activity (e.g., to stimulate proliferation and/or activation of DR3 receptor expressing cells (e.g., T-cells)) or the ability to upregulate DR3 receptor expression on the surface of cells. Optionally, the antibodies of the invention will bind to the same epitope as at least one of the antibodies specifically referred to herein. Such epitope binding can be routinely determined using assays known in the art.

The present invention also provides for antibodies (including molecules comprising, or alternatively consisting of, antibody fragments or variants thereof), that inhibit one or more DR3 receptor polypeptide mediated biological activities. In one embodiment, an antibody that inhibits one or more DR3 receptor polypeptide mediated biological activities comprises, or alternatively consists of at least one of the VH and/or VL domains of a heavy chain and/or light chain, respectively, referred to in Table 1, or fragment or variant thereof. In a specific embodiment, an antibody that inhibits one or more DR3 receptor polypeptide mediated biological activities comprises, or alternatively consists of at least one of the VH and VL domains of a heavy chain and a light chain, respectively, referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that inhibit proliferation of DR3 receptor expressing cells, for example T-cells. In one embodiment, an antibody that inhibits proliferation of DR3 receptor expressing cells comprises, or alternatively consists of at least one of the VH and/or VL domains of a heavy chain and/or a light chain, respectively, referred to in Table 1, or fragment or variant thereof. In a specific embodiment, an antibody that inhibits proliferation of DR3 receptor expressing cells comprises, or alternatively consists of at least one of the VH and VL domains of a heavy chain and/or a light chain, respectively, referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that inhibit activation of DR3 receptor expressing cells (e.g., T-cells). In one embodiment, an antibody that inhibits activation of DR3 receptor expressing cells comprises, or alternatively consists of at least one of the VH and/or VL domains of a heavy chain and/or a light chain, respectively, referred to in Table 1, or fragment or variant thereof. In a specific embodiment, an antibody that inhibits differentiation of DR3 receptor expressing cells comprises, or alternatively consists of at least one of the VH and VL domains of a heavy chain and/or a light chain, respectively, referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that inhibit T lymphocyte infiltration. In one embodiment, an antibody that inhibits T lymphocyte infiltration comprises, or alternatively consists of at least one of the VH and/or VL domains of a heavy chain and/or a light chain, respectively, referred to in Table 1, or fragment or variant thereof. In a specific embodiment, an antibody that inhibits T lymphocyte infiltration comprises, or alternatively consists of at least one of the VH and VL domains of a heavy chain and/or a light chain, respectively, referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that block or inhibit the binding of DR3 receptor ligand to a DR3 receptor polypeptide. In one embodiment, an antibody that blocks or inhibits the binding of DR3 receptor ligand to a DR3 receptor polypeptide comprises, or alternatively consists of at least one of the VH and VL domains of a heavy chain and/or a light chain, respectively, referred to in Table 1, or fragment or variant thereof. In a specific embodiment, an antibody that blocks or inhibits the binding of DR3 receptor ligand to a DR3 receptor polypeptide comprises, or alternatively consists of at least one of the VH and VL domains of a heavy chain and/or a light chain, respectively, referred to in Table 1, or fragment or variant thereof. In one specific embodiment the DR3 receptor ligand is TL1A. Nucleic acid molecules encoding these antibodies are also encompassed by the invention.

The present invention also provides for antibodies (including molecules comprising, or alternatively consisting of, antibody fragments or variants thereof), that stimulate proliferation of DR3 receptor expressing cells (e.g., T-cells). In one embodiment, an antibody that stimulates proliferation of DR3 receptor expressing cells comprises, or alternatively consists of at least one of the VH and VL domains of a heavy chain and/or a light chain, respectively, referred to in Table 1, or fragment or variant thereof. In a specific embodiment, an antibody that stimulates proliferation of DR3 receptor expressing cells comprises, or alternatively consists of at least one of the VH and VL domains of a heavy chain and/or a light chain, respectively, referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that stimulate activation of DR3 receptor expressing cells (e.g., T-cells). In one embodiment, an antibody that stimulates activation of DR3 receptor expressing cells comprises, or alternatively consists of at least one of the VH and VL domains of a heavy chain and/or a light chain, respectively, referred to in Table 1, or fragment or variant thereof. In a specific embodiment, an antibody that stimulates activation of DR3 receptor expressing cells comprises, or alternatively consists of at least one of the VH and VL domains of a heavy chain and/or a light chain, respectively, referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that stimulate T-lymphocyte infiltration. In one embodiment, an antibody that stimulates T-lymophocyte infiltration comprises, or alternatively consists of at least one of the VH and VL domains of a heavy chain and/or a light chain, respectively, referred to in Table 1, or fragment or variant thereof. In a specific embodiment, an antibody that stimulates T-lymphocyte infiltration comprises, or alternatively consists of at least one of the VH and VL domains of a heavy chain and/or a light chain, respectively, referred to in Table 1, or fragment or variant thereof. Nucleic acid molecules encoding these antibodies are also encompassed by the invention.

Antibodies of the present invention (including antibody fragments or variants thereof) may be characterized in a variety of ways. In particular, antibodies and related molecules of the invention may be assayed for the ability to specifically bind to DR3 receptor polypeptides or a fragment or variant of DR3 receptor polypeptides using techniques described herein or routinely modifying techniques known in the art. Assays for the ability of the antibodies of the invention to specifically bind DR3 receptor polypeptides or a fragment of DR3 receptor polypeptides may be performed in solution (e.g., Houghten, Bio/Techniques 13:412-421 (1992)), on beads (e.g., Lam, Nature 354:82-84 (1991)), on chips (e.g., Fodor, Nature 364:555-556 (1993)), on bacteria (e.g., U.S. Pat. No. 5,223,409), on spores (e.g., U.S. Pat. Nos. 5,571,698; 5,403,484; and 5,223,409), on plasmids (e.g., Cull et al., Proc. Natl. Acad. Sci. USA 89:1865-1869 (1992)) or on phage (e.g., Scott and Smith, Science 249:386-390 (1990); Devlin, Science 249:404-406 (1990); Cwirla et al., Proc. Natl. Acad. Sci. USA 87:7178-7182 (1990); and Felici, J. Mol. Biol. 222:301-310 (1991)) (each of these references is incorporated herein in its entirety by reference). Antibodies that have been identified to specifically bind to DR3 receptor polypeptides or a fragment or variant of DR3 receptor polypeptides can then be assayed for their specificity and affinity for DR3 receptor polypeptides or a fragment of DR3 receptor polypeptides using or routinely modifying techniques described herein or otherwise known in the art.

The antibodies of the invention may be assayed for specific binding to DR3 receptor polypeptides and cross-reactivity with other antigens by any method known in the art. Immunoassays which can be used to analyze specific binding and cross-reactivity include, but are not limited to, competitive and non-competitive assay systems using techniques such as BIAcore analysis, FACS (fluorescence activated cell sorter) analysis, immunofluorescence, immunocytochemistry, radioimmunoassays, ELISA (enzyme linked immunosorbent assay), “sandwich” immunoassays, immunoprecipitation assays, western blots, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

ELISAs comprise preparing antigen, coating the well of a 96-well microtiter plate with the antigen, washing away antigen that did not bind the wells, adding the antibody of interest conjugated to a detectable compound such as an enzymatic substrate (e.g., horseradish peroxidase or alkaline phosphatase) to the wells and incubating for a period of time, washing away unbound antibodies or non-specifically bound antibodies, and detecting the presence of the antibodies specifically bound to the antigen coating the well. In ELISAs, the antibody of interest does not have to be conjugated to a detectable compound; instead, a second antibody (which recognizes the antibody of interest) conjugated to a detectable compound may be added to the well. Alternatively, the antigen need not be directly coated to the well; instead the ELISA plates may be coated with an anti-Ig Fc antibody, and the antigen in the form or a DR3-Fc fusion protein, may be bound to the anti-Ig Fc coated to the plate. This may be desirable so as to maintain the antigen protein (e.g., the DR3 receptor polypeptides) in a more native conformation than it may have when it is directly coated to a plate. In another alternative, instead of coating the well with the antigen, the antibody may be coated to the well. In this case, the detectable molecule could be the antigen conjugated to a detectable compound such as an enzymatic substrate (e.g., horseradish peroxidase or alkaline phosphatase). One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The binding affinity of an antibody (including an scFv or other molecule comprising, or alternatively consisting of, antibody fragments or variants thereof) to an antigen and the off-rate of an antibody-antigen interaction can be determined by competitive binding assays. One example of a competitive binding assay is a radioimmunoassay comprising the incubation of labeled antigen (e.g., antigen labeled with ³H or ¹²⁵I), or fragment or variant thereof with the antibody of interest in the presence of increasing amounts of unlabeled antigen, and the detection of the antibody bound to the labeled antigen. The affinity of the antibody of the present invention for DR3 receptor and the binding off-rates can be determined from the data by Scatchard plot analysis. Competition with a second antibody can also be determined using radioimmunoassays. In this case, DR3 receptor polypeptide is incubated with an antibody of the present invention conjugated to a labeled compound (e.g., compound labeled with ³H or ¹²⁵I) in the presence of increasing amounts of an unlabeled second anti-DR3 receptor antibody. This kind of competitive assay between two antibodies, may also be used to determine if two antibodies bind the same or different epitopes.

In a preferred embodiment, BIAcore kinetic analysis is used to determine the binding on and off rates of antibodies (including antibody fragments or variants thereof) to a DR3 receptor, or fragments of a DR3 receptor. BIAcore kinetic analysis comprises analyzing the binding and dissociation of antibodies from chips with immobilized DR3 receptors on their surface.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40 degrees C., adding protein A and/or protein G sepharose beads to the cell lysate, incubating for about an hour or more at 40 degrees C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1.

### Antibody Conjugates and Fusion Proteins

The present invention encompasses antibodies (including antibody fragments or variants thereof), recombinantly fused or chemically conjugated (including both covalent and non-covalent conjugations) to molecules including, but not limited to, polymers, heterologous polypeptides, marker sequences, diagnostic agents and/or therapeutic agents. Additionally, the present invention encompasses antibodies (including antibody fragments or variants thereof), modified by natural processes, such as posttranslational processing, or by chemical modification techniques, which are well known in the art and discussed further herein.

In a specific embodiment, the present invention encompasses antibodies (including antibody fragments or variants thereof), that are chemically modified. This chemical modification may provide additional advantages such as increased solubility, stability and circulating time of the molecule, or decreased immunogenicity. The chemical moieties for derivitization may be selected from water soluble polymers such as polyethylene glycol, ethylene glycol/propylene glycol copolymers, carboxymethycellulose, dextran, polyvinyl alcohol and the like. The antibodies may be modified at random positions within the molecule, or at predetermined positions within the molecule and may include one, two, three, or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the antibody with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to antibodies via linkage to any of a number of amino acid residues. For example, polyethylene glycol can be linked to a proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire antibodies chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the antibody at the N-terminus with a carbonyl group containing polymer is achieved.

As indicated above, pegylation of the antibodies of the invention may be accomplished by any number of means. For example, polyethylene glycol may be attached to the antibody either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of antibodies without an intervening linker employs tresylated MPEG, which is produced by the modification of monmethoxy polyethylene glycol (MPEG) using tresylchloride (ClSO₂CH₂CF₃). Upon reaction of protein with tresylated MPEG, polyethylene glycol is directly attached to amine groups of the protein. Thus, the invention includes protein-polyethylene glycol conjugates produced by reacting antibodies of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p nitrophenolcarbonate, and various MPEG-succinate derivatives. A number additional polyethylene glycol derivatives and reaction chemistries for attaching polyethylene glycol to proteins are described in WO 98/32466, the entire disclosure of which is incorporated herein by reference. Pegylated protein products produced using the reaction chemistries set out herein are included within the scope of the invention.

The number of polyethylene glycol moieties attached to each anti-DR3 receptor antibody (i.e., the degree of substitution) may also vary. For example, the pegylated antibodies of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

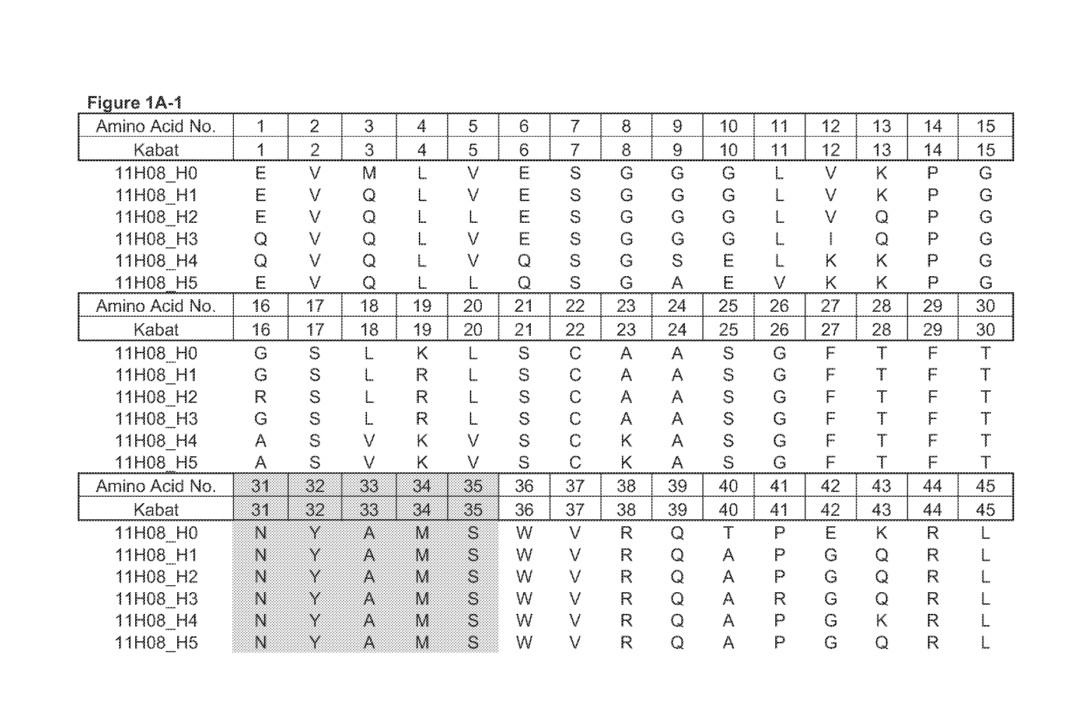

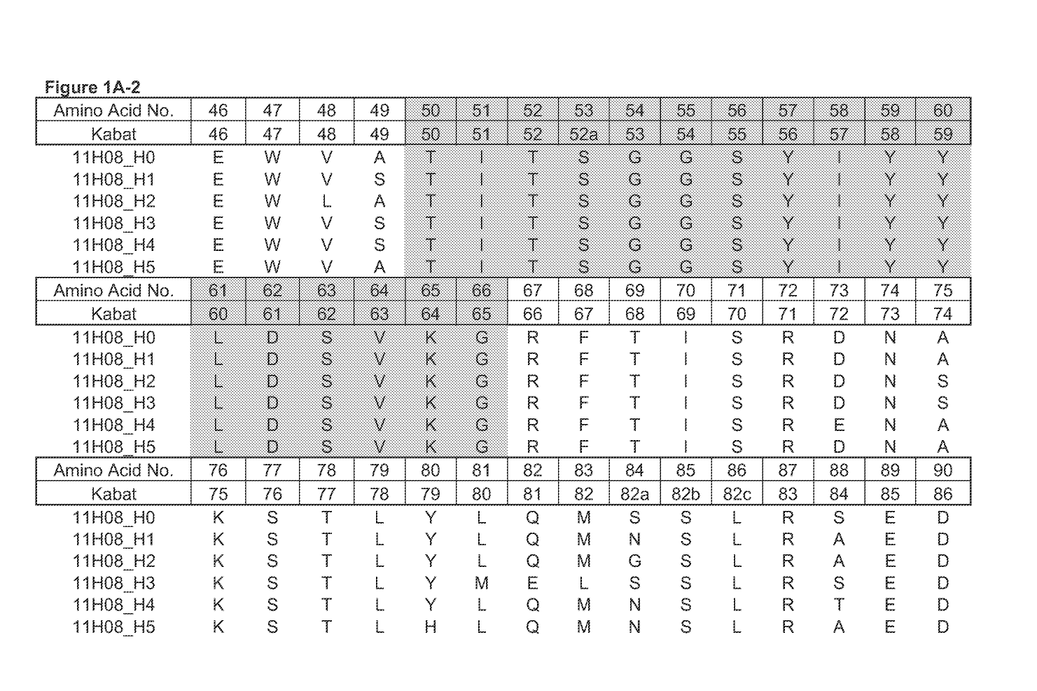

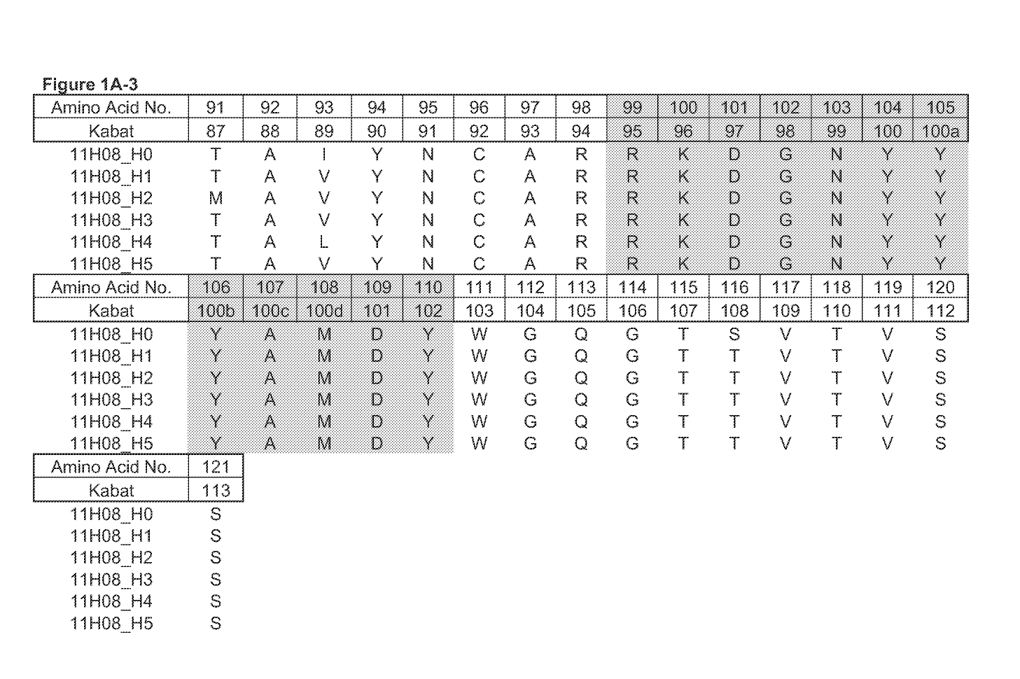

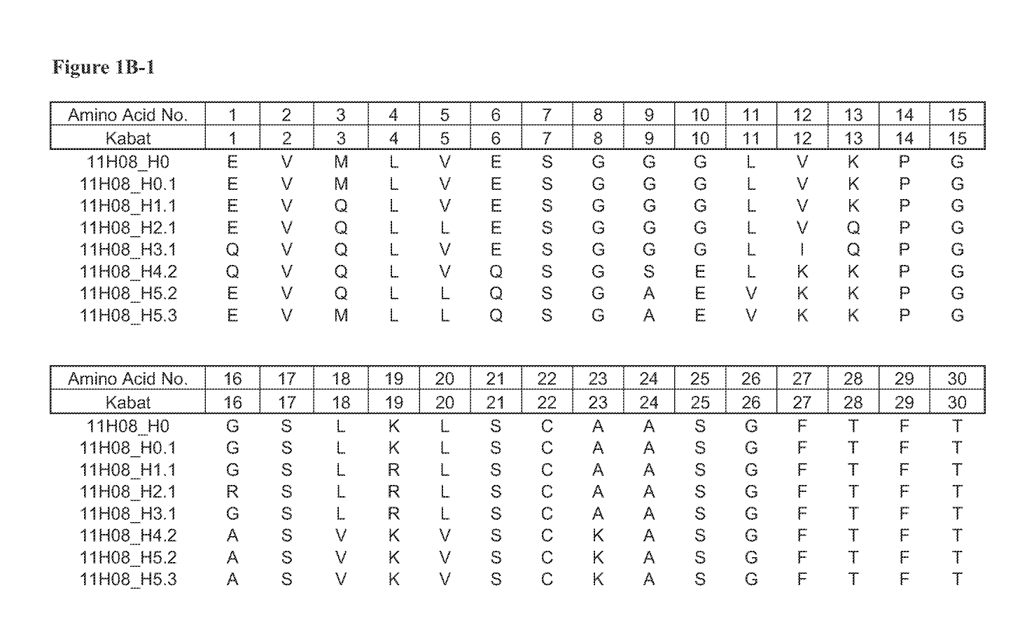

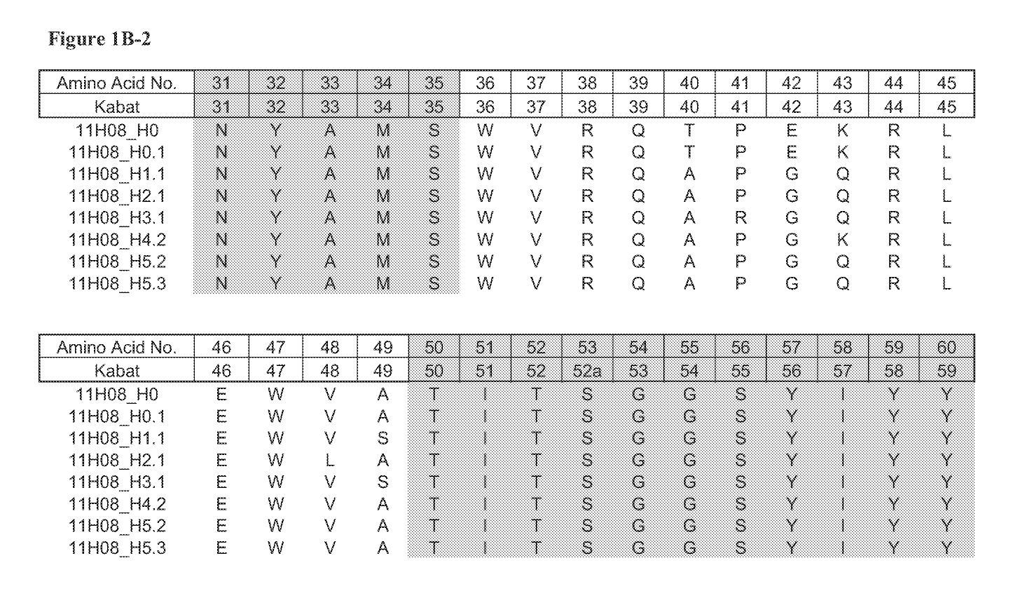

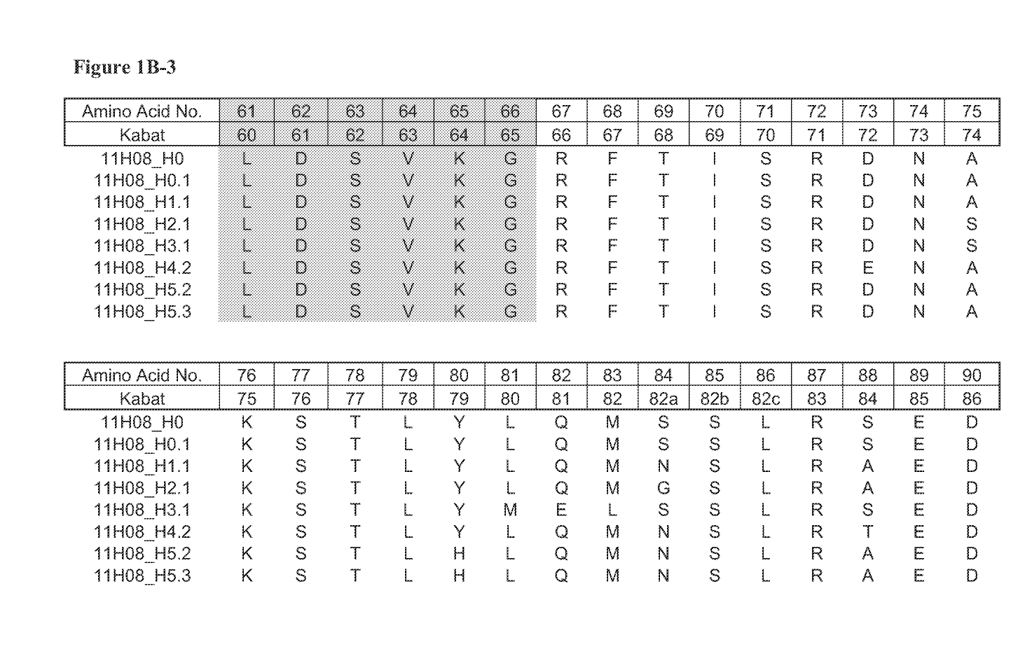

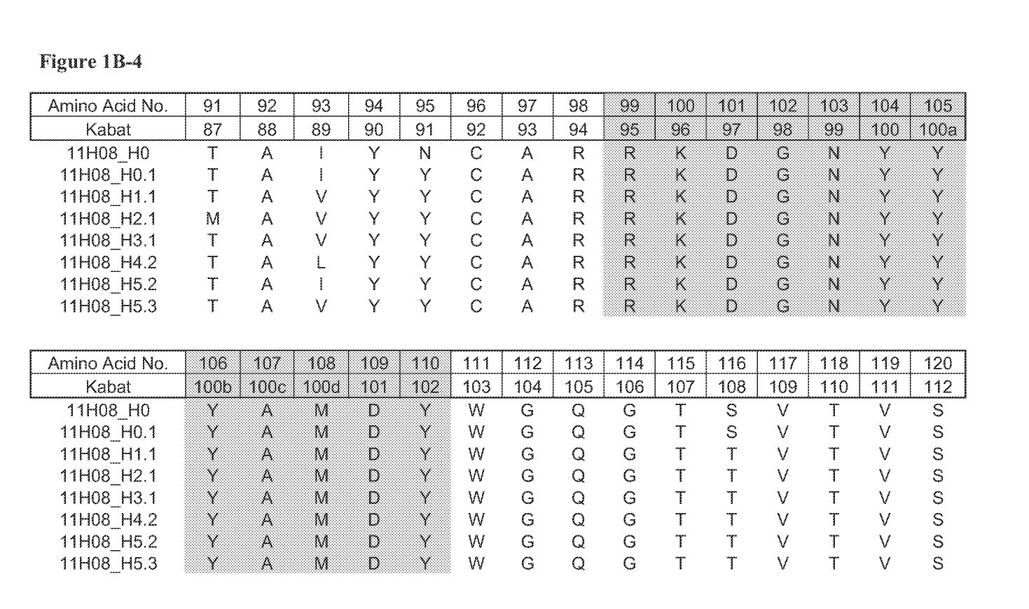

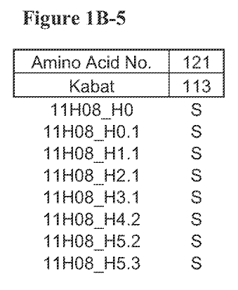

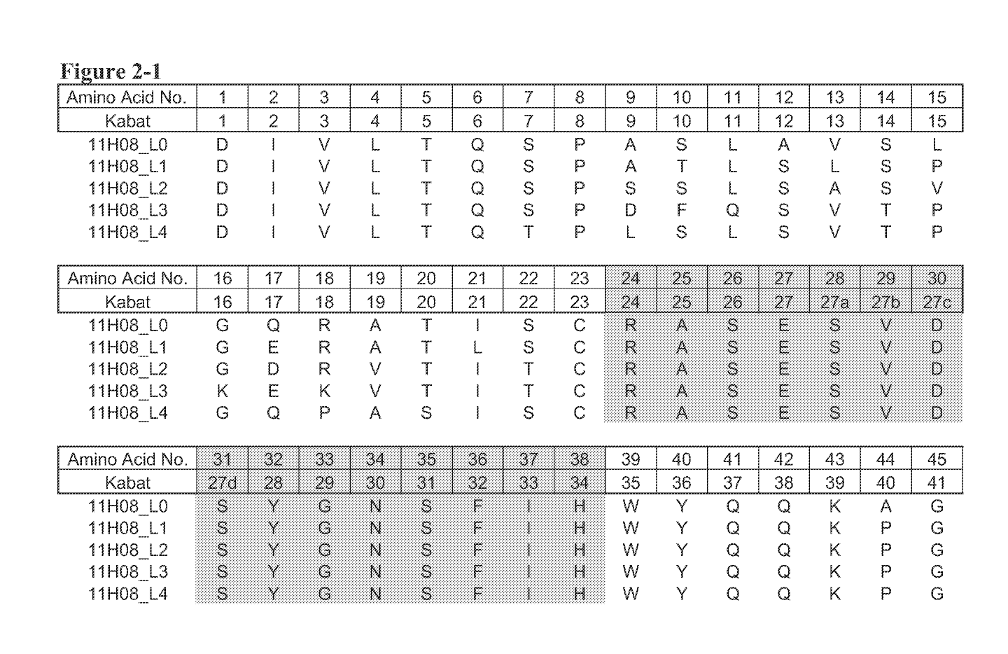

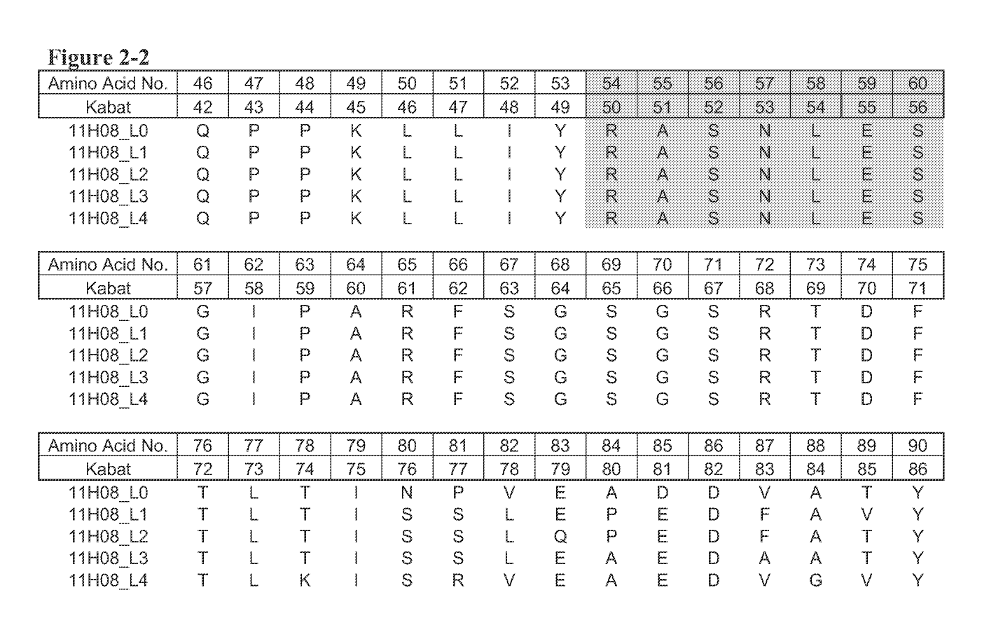

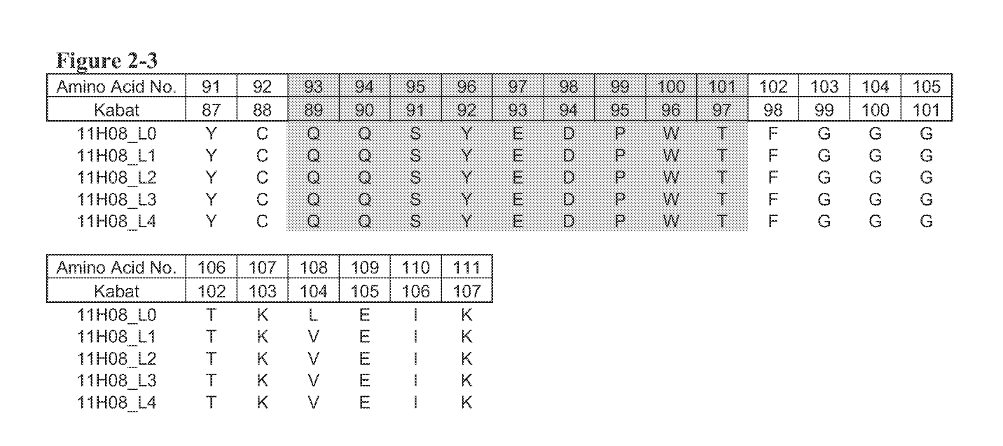

The present invention encompasses antibodies (including antibody fragments or variants thereof), recombinantly fused or chemically conjugated (including both covalent and non-covalent conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. For example, antibodies of the invention may be used to target heterologous polypeptides to particular cell types (e.g., cancer cells), either in vitro or in vivo, by fusing or conjugating the heterologous polypeptides to antibodies of the invention that are specific for particular cell surface antigens or which bind antigens that bind particular cell surface receptors. Antibodies of the invention may also be fused to albumin (including but not limited to recombinant human serum albumin (see, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)), resulting in chimeric polypeptides. In a preferred embodiment, antibodies of the present invention (including fragments or variants thereof) are fused with polypeptide fragments comprising, or alternatively consisting of, amino acid residues of human serum albumin. In a preferred embodiment, antibodies of the present invention (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety.

In addition, as described in U.S. Pat. No. 7,521,424, which is incorporated by reference in its entirety, fragments of serum albumin polypeptides corresponding to an albumin protein portion of an albumin fusion protein, include the full length protein as well as polypeptides having one or more residues deleted from the amino terminus of the amino acid sequence of the reference polypeptide (i.e., serum albumin, or a serum albumin portion of an albumin fusion protein). In preferred embodiments, N-terminal deletions may be described by the general formula m to 585, where 585 is a whole integer representing the total number of amino acid residues in mature human serum albumin (SEQ ID NO:4), and m is defined as any integer ranging from 2 to 579. Polynucleotides encoding these polypeptides are also encompassed by the invention.

In addition, as described in U.S. Pat. No. 7,521,424, which is incorporated by reference in its entirety, the present invention provides polypeptides having one or more residues deleted from the carboxy terminus of the amino acid sequence of an albumin protein corresponding to an albumin protein portion of an albumin fusion protein (e.g., serum albumin or an albumin protein portion of an albumin fusion protein). In particular, C-terminal deletions may be described by the general formula I to n, where n is any whole integer ranging from 6 to 584, where 584 is the whole integer representing the total number of amino acid residues in mature human serum albumin (SEQ ID NO:4) minus 1. Polynucleotides encoding these polypeptides are also encompassed by the invention.

Antibodies of the present invention (including fragments or variants thereof) may be fused to either the N- or C-terminal end of the heterologous polypeptide (e.g., human serum albumin polypeptide). Heterologous polypeptides may be fused to the heavy chain or light chain constant domains of the antibodies of the invention. In a preferred embodiment, the heterologous polypeptide is fused to the CH1 or Cκ domains. In another preferred embodiment, the heterologous polypeptide is fused to the CH1 domain. In a preferred embodiment, the heterologous polypeptide is from human serum albumin. Polynucleotides encoding fusion proteins of the invention are also encompassed by the invention. Such fusion proteins may, for example, facilitate purification and may increase half-life in vivo. Antibodies fused or conjugated to heterologous polypeptides may also be used in in vitro immunoassays and purification methods using methods known in the art. See e.g., Harbor et al., supra, and PCT publication WO 93/2 1232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991), which are incorporated by reference in their entireties.

The present invention further includes compositions comprising, or alternatively consisting of, heterologous polypeptides fused or conjugated to antibody fragments. For example, the heterologous polypeptides may be fused or conjugated to a Fab fragment, Fd fragment, Fv fragment, F(ab)₂ fragment, or a portion thereof. Methods for fusing or conjugating polypeptides to antibody portions are known in the art. See, e.g., U.S. Pat. Nos. 5,356,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88: 10535-10539 (1991); Zheng et al., J. Immunol. 154:5590-5600 (1995); and Vil et al., Proc. Natl. Acad. Sci. USA 89:11357-11341 (1992) (said references incorporated by reference in their entireties).

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of antibodies (including molecules comprising, or alternatively consisting of, antibody fragments or variants thereof), such methods can be used to generate antibodies with altered activity (e.g., antibodies with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-35 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, polynucleotides encoding antibodies of the invention may be altered by being subjected to random mutagenesis by error-prone PCR, random nucleotide insertion or other methods prior to recombination. In another embodiment, one or more portions of a polynucleotide encoding an antibody which portions specifically bind to DR3 receptor may be recombined with one or more components, motifs, sections, parts, domains, fragments, etc. of one or more heterologous molecules.

Moreover, the antibodies of the present invention (including antibody fragments or variants thereof), can be fused to marker sequences, such as a polypeptides to facilitate purification. In preferred embodiments, the marker amino acid sequence is a hexa-histidine polypeptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., Cell 37:767 (1984)) and the FLAG® tag (Stratagene, La Jolla, Calif.).

The present invention further encompasses antibodies (including antibody fragments or variants thereof), conjugated to a diagnostic or therapeutic agent. The antibodies can be used diagnostically to, for example, monitor or prognose the development or progression of a tumor as part of a clinical testing procedure to, e.g., determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include, but are not limited to, horseradish peroxidase, alkaline phosphatase, beta-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin; and examples of suitable radioactive material include, but are not limited to, iodine (¹²¹I, ¹²³I, ¹²⁵I, ¹³¹I), carbon (¹⁴C), sulfur (³⁵S), tritium (³H), indium (¹¹¹In, ¹¹²In, ^{113m}In, ^{115m}In), technetium (⁹⁹Tc, ^{99m}Tc), thallium (201Ti), gallium (⁶⁸Ga, ⁶⁷Ga), palladium (¹⁰³Pd), molybdenum (⁹⁹Mo), xenon (¹³⁵Xe), fluorine (18F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

Further, an antibody of the invention (including an scFv or other molecule comprising, or alternatively consisting of, antibody fragments or variants thereof), may be coupled or conjugated to a therapeutic moiety such as a cytotoxin, e.g., a cytostatic or cytocidal agent, a therapeutic agent or a radioactive metal ion, e.g., alpha-emitters such as, for example, ²¹³Bi, or other radioisotopes such as, for example, 103Pd, ¹³⁵Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ⁹⁰Y, ¹¹⁷Tin, ¹⁸⁶Re, ¹⁸⁸Re and ¹⁶⁶Ho. In specific embodiments, an antibody or fragment thereof is attached to macrocyclic chelators that chelate radiometal ions, including but not limited to, ¹⁷⁷Lu, ⁹⁰Y, ¹⁶⁶Ho, and ¹⁵³Sm, to polypeptides. In specific embodiments, the macrocyclic chelator is 1,4,7,10-tetraazacyclododecane-N,N′,N″,N′″-tetraacetic acid (DOTA). In other specific embodiments, the DOTA is attached to the an antibody of the invention or fragment thereof via a linker molecule. Examples of linker molecules useful for conjugating DOTA to a polypeptide are commonly known in the art—see, for example, DeNardo et al., Clin Cancer Res. 4(10):2483-90, 1998; Peterson et al., Bioconjug. Chem. 10(4):553-7, 1999; and Zimmerman et al., Nucl. Med. Biol. 26(8):943-50, 1999 which are hereby incorporated by reference in their entirety.

A cytotoxin or cytotoxic agent includes any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and fragments, variants or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques known in the art may be applied to label antibodies of the invention. Such techniques include, but are not limited to, the use of bifunctional conjugating agents (see e.g., U.S. Pat. Nos. 5,756,065; 5,714,711; 5,696,239; 5,652,371; 5,505,931; 5,489,425; 5,435,990; 5,428,139; 5,342,604; 5,274,119; 4,994,560; and 5,808,003; the contents of each of which are hereby incorporated by reference in its entirety) and direct coupling reactions (e.g., Bolton-Hunter and Chloramine-T reaction).

The antibodies of the invention which are conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, but are not limited to, for example, a toxin such as abrin, ricin A, alpha toxin, pseudomonas exotoxin, or diphtheria toxin, saporin, momordin, gelonin, pokeweed antiviral protein, alpha-sarcin and cholera toxin; a protein such as tumor necrosis factor, alpha-interferon, beta-interferon, nerve growth factor, platelet derived growth factor, tissue plasminogen activator, an apoptotic agent, e.g., TNF-alpha (TNF-α), TNF-beta, AIM I (see, International Publication No. WO 97/35899), AIM II (see, International Publication No. WO 97/34911), Fas Ligand (Takahashi et al., Int. Immunol., 6:1567-1574 (1994)), VEGI (see, International Publication No. WO 99/23105), a thrombotic agent or an anti-angiogenic agent, e.g., angiostatin or endostatin; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Antibodies of the invention (including antibody fragments or variants thereof), may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating a therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody of the invention can be conjugated to a second antibody to form an antibody heteroconjugate as described by Segal in U.S. Pat. No. 4,676,980, which is incorporated herein by reference in its entirety.

An antibody of the invention (including an other molecules comprising, or alternatively consisting of, an antibody fragment or variant thereof), with or without a therapeutic moiety conjugated to it, administered alone or in combination with cytotoxic factor(s) and/or cytokine(s) can be used as a therapeutic.

Additionally, antibodies of the invention may be modified by post-translational modifications including but not limited to, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of procaryotic host cell expression.

Antibodies may be modified by natural processes, such as posttranslational processing, or by chemical modification techniques, which are well known in the art. It will be appreciated that the same type of modification may be present in the same or varying degrees at several sites in a given antibody. Also, a given antibody may contain many types of modifications.

Modifications may include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

### Uses of Antibodies of the Invention

Antibodies of the present invention may be used, for example, but not limited to, to purify, detect, and target DR3 polypeptides, including both in vitro and in vivo diagnostic and therapeutic methods. For example, the antibodies have use in immunoassays for qualitatively and quantitatively measuring levels of DR3 receptor polypeptides in biological samples. See, e.g., Harlow et al., Antibodies: A Laboratory Manual, (Cold Spring Harbor Laboratory Press, 2nd ed. 1988) (incorporated by reference herein in its entirety).

### Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types, such as T cells (e.g., activated T cells). In other embodiments, the antibodies of the invention may be useful as tumor and/or cancer cell markers. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

### Epitope Mapping

The present invention provides antibodies (including antibody fragments or variants thereof), that can be used to identify epitopes of a DR3 receptor polypeptide. In particular, the antibodies of the present invention can be used to identify epitopes of a human DR3 receptor polypeptide (e.g., SEQ ID NO:2) or a DR3 receptor polypeptide expressed on human cells; a murine DR3 receptor or a DR3 receptor polypeptide expressed on murine cells; a rat DR3 receptor polypeptide receptor or a DR3 receptor polypeptide expressed on rat cells; or a monkey DR3 receptor polypeptide or a DR3 receptor polypeptide expressed on monkey cells, using techniques described herein or otherwise known in the art. Fragments which function as epitopes may be produced by any conventional means. (See, e.g., Houghten, Proc. Natl. Acad. Sci. USA 82:5131-5135 (1985), further described in U.S. Pat. No. 4,711,211.) Identified epitopes of antibodies of the present invention may, for example, be used as vaccine candidates, i.e., to immunize an individual to elicit antibodies against the naturally occurring forms of DR3 receptor polypeptides.

### Diagnostic Uses of Antibodies

Labeled antibodies of the invention (including molecules comprising, or alternatively consisting of, antibody fragments or variants thereof) which specifically bind to a DR3 receptor polypeptide can be used for diagnostic purposes to detect, diagnose, prognose, or monitor diseases and/or disorders. In specific embodiments, labeled antibodies of the invention (including molecules comprising, or alternatively consisting of, antibody fragments or variants thereof) which specifically bind to a DR3 receptor polypeptide can be used for diagnostic purposes to detect, diagnose, prognose, or monitor diseases and/or disorders associated with the aberrant expression and/or activity of a DR3 receptor polypeptide.

The invention provides for the detection of expression of a DR3 receptor polypeptide comprising: (a) assaying the expression of a DR3 receptor polypeptide in a biological sample from an individual using one or more antibodies of the invention that specifically binds to a DR3 receptor polypeptide; and (b) comparing the level of a DR3 receptor polypeptide with a standard level of a DR3 receptor polypeptide, (e.g., the level in normal biological samples).

The invention provides for the detection of aberrant expression of a DR3 receptor polypeptide comprising: (a) assaying the expression of a DR3 receptor polypeptide in a biological sample from an individual using one or more antibodies of the invention that specifically binds to a DR3 receptor polypeptide; and (b) comparing the level of a DR3 receptor polypeptide with a standard level of a DR3 receptor polypeptide, e.g., in normal biological samples, whereby an increase or decrease in the assayed level of a DR3 receptor polypeptide compared to the standard level of a DR3 receptor polypeptide is indicative of aberrant expression.

By “biological sample” is intended any fluids and/or cells obtained from an individual, body fluid, body tissue, body cell, cell line, tissue culture, or other source which may contain a DR3 receptor polypeptide protein or mRNA. Body fluids include, but are not limited to, sera, plasma, urine, synovial fluid, spinal fluid, saliva, and mucous. Tissues samples may be taken from virtually any tissue in the body. Tissue samples may also be obtained from autopsy material. Methods for obtaining tissue biopsies and body fluids from mammals are well known in the art. Where the biological sample is to include mRNA, a tissue biopsy is the preferred source.

One aspect of the invention is the detection and diagnosis of a disease or disorder associated with aberrant expression of a DR3 receptor polypeptide in an animal, preferably a mammal and most preferably a human. In one embodiment, diagnosis comprises: a) administering (for example, parenterally, subcutaneously, or intraperitoneally) to a subject an effective amount of a labeled antibody of the invention (including molecules comprising, or alternatively consisting of, antibody fragments or variants thereof) that specifically binds to a DR3 receptor polypeptide; b) waiting for a time interval following the administering for permitting the labeled antibody to preferentially concentrate at sites in the subject where DR3 receptor polypeptide is expressed (and for unbound labeled molecule to be cleared to background level); c) determining background level; and d) detecting the labeled antibody in the subject, such that detection of labeled antibody or fragment thereof above the background level and above or below the level observed in a person without the disease or disorder indicates that the subject has a particular disease or disorder associated with aberrant expression of a DR3 receptor polypeptide or a DR3 receptor polypeptide receptor. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In one embodiment, monitoring of the disease or disorder is carried out by repeating the method for diagnosing the disease or disorder, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### Therapeutic Uses of Antibodies

One or more antibodies of the present invention (including molecules comprising, or alternatively consisting of, antibody fragments or variants thereof) that specifically bind to DR3 receptor may be used locally or systemically in the body as a therapeutic. The present invention is further directed to antibody-based therapies which involve administering antibodies of the invention (including molecules comprising, or alternatively consisting of, antibody fragments or variants thereof) to an animal, preferably a mammal, and most preferably a human, for preventing or treating one or more of the disclosed diseases, disorders, or conditions. Therapeutic compounds of the invention include, but are not limited to, antibodies of the invention and nucleic acids encoding antibodies (and anti-idiotypic antibodies) of the invention as described herein. In one embodiment, the antibodies of the invention can be used to treat, ameliorate or prevent diseases, disorders or conditions, including, but not limited to, any one or more of the diseases, disorders, or conditions described herein. The treatment and/or prevention of diseases, disorders, or conditions includes, but is not limited to, alleviating symptoms associated with those diseases, disorders or conditions. Antibodies of the invention may be provided in pharmaceutically acceptable compositions as known in the art or as described herein. In certain embodiments, properties of the antibodies of the present invention make the antibodies better therapeutic agents than previously described DR3 receptor binding antibodies. In highly preferred embodiments, antagonistic antibodies, or fragments thereof, of the invention that bind a DR3 receptor polypeptide can be used to treat inflammatory and autoimmune diseases, including but not limited to Crohn's disease, colitis, inflammatory bowel disease, allergy, asthma, arthritis, atherosclerosis, osteoporosis, bone cancer pain, and multiple sclerosis.

### Therapeutic Uses of Antibodies for Treating Autoimmune Disorders

In highly preferred embodiments, antibodies of the invention that bind a DR3 receptor polypeptide and inhibit differentiation of DR3 receptor expressing cells are used to treat, prevent or ameliorate autoimmune diseases, disorders, or conditions associated with such diseases or disorders. In specific embodiments, antibodies of the invention are used to inhibit the progression of an autoimmune response and other related disorders. Antagonistic antibodies, of the present invention may be useful in treating, preventing, and/or diagnosing diseases, disorders, and/or conditions of the immune system, by, for example, activating or inhibiting the proliferation, activation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune diseases, disorders, and/or conditions may be genetic, somatic, such as cancer and some autoimmune diseases, acquired (e.g., by chemotherapy or toxins), or infectious diseases. Moreover, Antibodies of the present invention can be used as a marker or detector of a particular immune system disease or disorder.

Furthermore, autoimmune disorders result from inappropriate recognition of self as foreign material by immune cells. This inappropriate recognition results in an immune response leading to the destruction of the host tissue. Therefore, the administration of antagonistic antibodies of the invention that can inhibit an immune response, particularly the proliferation, differentiation, or chemotaxis of T-cells or cells of monocytic lineage, may be an effective therapy in preventing autoimmune disorders.

Autoimmune diseases or disorders that may be treated, prevented, and/or diagnosed by antagonistic antibodies of the present invention include, but are not limited to, one or more of the following: multiple sclerosis, rheumatoid arthritis (often characterized, e.g., by immune complexes in joints), autoimmune hemolytic anemia, autoimmune neonatal thrombocytopenia, idiopathic thrombocytopenia purpura, autoimmunocytopenia, hemolytic anemia, antiphospholipid syndrome, dermatitis, allergic encephalomyelitis, myocarditis, relapsing polychondritis, rheumatic heart disease, Neuritis, Uveitis Ophthalmia, Polyendocrinopathies, Purpura (e.g., Henloch-Scoenlein purpura), Reiter's Disease, Stiff-Man Syndrome, Autoimmune Pulmonary Inflammation, Autism, Guillain-Barre Syndrome, insulin dependent diabetes mellitis, and autoimmune inflammatory eye disorders, autoimmune thyroiditis, hypothyroidism (i.e., Hashimoto's thyroiditis, systemic lupus erhythematosus, Goodpasture's syndrome, Pemphigus, Receptor autoimmunities such as, for example, (a) Graves' Disease, (b) Myasthenia Gravis, and (c) insulin resistance, autoimmune thrombocytopenic purpura, rheumatoid arthritis, schleroderma with anti-collagen antibodies, mixed connective tissue disease, polymyositis/dermatomyositis, pernicious anemia, idiopathic Addison's disease, infertility, glomerulonephritis such as primary glomerulonephritis and IgA nephropathy, bullous pemphigoid, Sjogren's syndrome, adrenergic drug resistance (including adrenergic drug resistance with asthma or cystic fibrosis), chronic active hepatitis, primary biliary cirrhosis, other endocrine gland failure, vitiligo, vasculitis, post-MI, cardiotomy syndrome, urticaria, atopic dermatitis, inflammatory myopathies, and other inflammatory, granulamatous, degenerative, and atrophic disorders.

Additional autoimmune disorders that may be treated, prevented, and/or diagnosed with the compositions of the invention include, but are not limited to scleroderma with anti-collagen antibodies (often characterized, e.g., by nucleolar and other nuclear antibodies), mixed connective tissue disease (often characterized, e.g., by antibodies to extractable nuclear antigens (e.g., ribonucleoprotein)), polymyositis (often characterized, e.g., by nonhistone ANA), pernicious anemia (often characterized, e.g., by antiparietal cell, microsomes, and intrinsic factor antibodies), idiopathic Addison's disease (often characterized, e.g., by humoral and cell-mediated adrenal cytotoxicity, infertility (often characterized, e.g., by antispermatozoal antibodies), glomerulonephritis (often characterized, e.g., by glomerular basement membrane antibodies or immune complexes), bullous pemphigoid (often characterized, e.g., by IgG and complement in basement membrane), Sjogren's syndrome (often characterized, e.g., by multiple tissue antibodies, and/or a specific nonhistone ANA (SS-B)), diabetes millitus (often characterized, e.g., by cell-mediated and humoral islet cell antibodies), and adrenergic drug resistance (including adrenergic drug resistance with asthma or cystic fibrosis) (often characterized, e.g., by beta-adrenergic receptor antibodies).

Additional autoimmune disorders (that are possible) that may be treated, prevented, and/or diagnosed with the compositions of the invention include, but are not limited to, chronic active hepatitis (often characterized, e.g., by smooth muscle antibodies), primary biliary cirrhosis (often characterized, e.g., by mitchondrial antibodies), other endocrine gland failure (often characterized, e.g., by specific tissue antibodies in some cases), vitiligo (often characterized, e.g., by melanocyte antibodies), vasculitis (often characterized, e.g., by Ig and complement in vessel walls and/or low serum complement), post-MI (often characterized, e.g., by myocardial antibodies), cardiotomy syndrome (often characterized, e.g., by myocardial antibodies), urticaria (often characterized, e.g., by IgG and IgM antibodies to IgE), atopic dermatitis (often characterized, e.g., by IgG and IgM antibodies to IgE), asthma (often characterized, e.g., by IgG and IgM antibodies to IgE), and many other inflammatory, granulamatous, degenerative, and atrophic disorders.

In a specific preferred embodiment, arthritis is treated, prevented, and/or diagnosed using antagonistic antibodies, of the present invention. In another specific preferred embodiment, Crohn's disease is treated, prevented, and/or diagnosed using antagonistic antibodies of the present invention. In another specific preferred embodiment, colitis is treated, prevented, and/or diagnosed using antagonistic antibodies of the present invention. In another specific preferred embodiment atherosclerosis is treated, prevented, and/or diagnosed using antagonistic antibodies of the present invention. In another specific preferred embodiment Multiple Sclerosis is treated, prevented, and/or diagnosed using antagonistic antibodies of the present invention. In a preferred embodiment, the autoimmune diseases and disorders and/or conditions associated with the diseases and disorders recited above are treated, prevented, and/or diagnosed using antibodies against the protein of the invention.

Similarly, allergic reactions and conditions, such as asthma (particularly allergic asthma) or other respiratory problems, may also be treated, prevented, and/or diagnosed using antagonistic antibodies of the invention. Moreover, these molecules can be used to treat, prevent, and/or diagnose anaphylaxis, hypersensitivity to an antigenic molecule, or blood group incompatibility.

### Therapeutic Uses of Antibodies for Treating Inflammatory Disorders

In particularly preferred embodiments, antagonistic antibodies of the present invention are used in the diagnosis, prognosis, prevention, and/or treatment of inflammatory conditions. For example, since antagonistic antibodies of the invention may inhibit the activation, proliferation and/or infiltration of cells involved in an inflammatory response, these molecules can be used to diagnose, prognose, prevent, and/or treat chronic and acute inflammatory conditions. Such inflammatory conditions include, but are not limited to, for example, inflammation associated with infection (e.g., septic shock, sepsis, or systemic inflammatory response syndrome), osteoporosis, bone cancer pain, ischemia-reperfusion injury, endotoxin lethality, complement-mediated hyperacute rejection, nephritis, cytokine or chemokine induced lung injury, inflammatory bowel disease, colitis, Crohn's disease, over production of cytokines (e.g., TNF or IL-1.), respiratory disorders (such as, e.g., asthma and allergy); gastrointestinal disorders (such as, e.g., inflammatory bowel disease); cancers (such as, e.g., gastric, ovarian, lung, bladder, liver, and breast); CNS disorders (such as, e.g., multiple sclerosis; ischemic brain injury and/or stroke; traumatic brain injury; neurodegenerative disorders, such as, e.g., Parkinson's disease and Alzheimer's disease; AIDS-related dementia; and prion disease); cardiovascular disorders (such as, e.g., atherosclerosis, myocarditis, cardiovascular disease, and cardiopulmonary bypass complications); as well as many additional diseases, conditions, and disorders that are characterized by inflammation (such as, e.g., hepatitis, rheumatoid arthritis, collagen-induced arthritis, gout, trauma, septic shock, pancreatitis, sarcoidosis, dermatitis, renal ischemia-reperfusion injury, Grave's disease, systemic lupus erythematosis, diabetes mellitus, atherosclerosis, and allogenic transplant rejection).

Because inflammation is a fundamental defense mechanism, inflammatory disorders can effect virtually any tissue of the body. Therefore, in particularly preferred embodiments, antagonistic antibodies of the invention are used in the treatment of tissue-specific inflammatory disorders, including, but not limited to, adrenalitis, alveolitis, angiocholecystitis, appendicitis, atherosclerosis, balanitis, blepharitis, bronchitis, bursitis, carditis, cellulitis, cervicitis, cholecystitis, chorditis, cochlitis, colitis, conjunctivitis, cystitis, dermatitis, diverticulitis, encephalitis, endocarditis, esophagitis, eustachitis, fibrositis, folliculitis, gastritis, gastroenteritis, gingivitis, glossitis, hepatosplenitis, keratitis, labyrinthitis, laryngitis, lymphangitis, mastitis, media otitis, meningitis, metritis, mucitis, myocarditis, myosititis, myringitis, nephritis, neuritis, orchitis, osteochondritis, otitis, pericarditis, peritendonitis, peritonitis, pharyngitis, phlebitis, poliomyelitis, prostatitis, pulpitis, retinitis, rhinitis, salpingitis, scleritis, sclerochoroiditis, scrotitis, sinusitis, ankylosing spondylitis, steatitis, stomatitis, synovitis, syringitis, tendonitis, tonsillitis, urethritis, and vaginitis.

In specific embodiments, antagonistic antibodies, or fragments thereof, are useful to treat, diagnose, and/or prevent transplantation rejections, graft-versus-host disease, experimental allergic and hyperacute xenograft rejection. Organ rejection occurs by host immune cell destruction of the transplanted tissue through an immune response. Similarly, an immune response is also involved in GVHD, but, in this case, the foreign transplanted immune cells destroy the host tissues. Antagonistic antibodies of the invention that inhibit an immune response, particularly the activation, proliferation, infiltration, or chemotaxis of T-cells or cells of monocytic lineage, may be an effective therapy in preventing organ rejection or GVHD.

### Therapeutic Uses of Antibodies for Treating Cancers

In highly preferred embodiments, antibodies, fragments or variants thereof of the invention that bind a DR3 receptor polypeptide and inhibit proliferation of DR3 receptor expressing cells are used to treat, prevent or ameliorate cancer. Diseases associated with increased cell survival or the inhibition of apoptosis that could be treated, prevented, diagnosed, and/or prognosed using antibodies, fragments or variants thereof of the present invention, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer)

In preferred embodiments, antibodies, fragments or variants thereof of the invention are used to inhibit growth, progression, and/or metasis of cancers, in particular those listed above.

Additional diseases or conditions associated with increased cell survival that could be treated or detected by antibodies, fragments or variants thereof, or agonists or antagonists of the present invention include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Hyperproliferative diseases and/or disorders that could be detected and/or treated by antibodies, fragments or variants thereof of the invention, include, but are not limited to neoplasms located in the: liver, abdomen, bone, breast, digestive system, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by antibodies, fragments or variants thereof of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

In a specific embodiment, antibodies, fragments or variants thereof, of the present invention may be used to treat, diagnose, and/or prevent (1) cancers or neoplasms and (2) immune cell or tissue-related cancers or neoplasms. In a preferred embodiment, antibodies, fragments or variants thereof, of the present invention conjugated to a toxin or a radioactive isotope, as described herein, may be used to treat, diagnose, and/or prevent acute myelogeneous leukemia. In a further preferred embodiment, antibodies, fragments or variants thereof, of the present invention conjugated to a toxin or a radioactive isotope, as described herein, may be used to treat, diagnose, and/or prevent, chronic myelogeneous leukemia, multiple myeloma, non-Hodgkins lymphoma, and/or Hodgkins disease.

### Additional Therapeutic Uses of Antibodies

Additionally, antagonistic antibodies may be used to treat or prevent IgE-mediated allergic reactions. Such allergic reactions include, but are not limited to, asthma, rhinitis, and eczema.

Antagonists of the invention would be expected to reverse many of the activities of the ligand described above as well as find clinical or practical application as:

As a means of regulating secreted cytokines that are elicited by activation of the DR3 receptor.

A means of blocking various aspects of immune responses to foreign agents or self. Examples include autoimmune disorders such as lupus, and arthritis, as well as immunoresponsiveness to skin allergies, inflammation, bowel disease, injury and pathogens.

A therapy for preventing the B cell proliferation and Ig secretion associated with autoimmune diseases such as idiopathic thrombocytopenic purpura, systemic lupus erythramatosus and MS.

An inhibitor of B and/or T cell migration in endothelial cells. This activity disrupts tissue architecture or cognate responses and is useful, for example in disrupting immune responses, and blocking sepsis.

An inhibitor of graft versus host disease or transplant rejection.

An inhibitor of osteoclast function to prevent osteoporosis or bone cancer pain.

An inhibitor of activation of cells of monocytic lineage such as macrophages or dendritic cells, to prevent autoimmune disease.

An inhibitor of osteoblast activation to prevent aberrant responses in anabolism of bone.

A therapy for B cell and/or T cell malignancies such as ALL, Hodgkins disease, non-Hodgkins lymphoma, Chronic lymphocyte leukemia, plasmacytomas, multiple myeloma, Burkitt's lymphoma, and EBV-transformed diseases.

A therapy for chronic hypergammaglobulinemeia evident in such diseases as monoclonalgammopathy of undetermined significance (MGUS), Waldenstrom's disease, related idiopathic monoclonalgammopathies, and plasmacytomas.

A therapy for decreasing cellular proliferation of Large B-cell Lymphomas.

A means of decreasing the involvement of B cells and Ig associated with Chronic Myelogenous Leukemia.

An immunosuppressive agent(s).

Administration of antagonist antibodies of the present invention may be used to modulate IgE concentrations in vitro or in vivo. These antibodies may be used to treat or prevent IgE-mediated allergic reactions including, but not limited to, asthma, rhinitis, and eczema.

The antibodies may be employed in a composition with a pharmaceutically acceptable carrier, e.g., as described herein.

The antagonist antibodies, fragments or variants thereof, may be employed for instance to inhibit polypeptide chemotaxis and activation of macrophages and their precursors, and of neutrophils, basophils, B lymphocytes and T lymphocytes, in certain auto-immune and chronic inflammatory and infective diseases. Examples of autoimmune diseases are described herein and include multiple sclerosis, and insulin-dependent diabetes. The antagonist antibodies, fragments or variants thereof, may also be employed to treat infectious diseases including silicosis, sarcoidosis, idiopathic pulmonary fibrosis by, for example, preventing the recruitment and activation of mononuclear phagocytes. They may also be employed to treat idiopathic hyper-eosinophilic syndrome by, for example, preventing eosinophil production and migration. The antagonist antibodies, fragments or variants thereof, may also be employed for treating atherosclerosis, for example, by preventing monocyte infiltration in the artery wall.

Antibodies, fragments or variants thereof against polypeptides of the invention may be employed to treat ARDS (acute respiratory distress syndrome).

Diseases associated with increased apoptosis that could be treated or detected by polynucleotides, polypeptides, antibodies, fragments or variants thereof, of the invention, include; neurodegenerative disorders (such as Alzheimer's disease, Parkinson's disease, Amyotrophic lateral sclerosis, Retinitis pigmentosa, Cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

Diseases associated with bone loss, bone thinning, and aberrant bone remodeling that could be treated or detected by polynucleotides, polypeptides, antibodies, fragments or variants thereof, of the present invention include: osteoporosis (e.g., juvenile, postmenopausal, senile, severe, glucocorticoid-induced, drug-induced, as a result of ethanol abuse, as a result of testosterone deficiency, as result of Vitamin D deficiency, or as a result of malnutrition), osteopenia, osteomalacia, Paget's disease, andynamic bone disease, osteoectasia with hyperphosphatasia, osteogenesis imperfect, osteopetroses, osteosclerosis, rickets, Kashin-Beck disease, brittle bone disease, Progressive osseous heteroplasia, and osteodystrophy. The polynucleotides, polypeptide, antibodies, fragments or variants thereof, of the present invention are useful as agents in the regulation and proliferation of oseteoclasts and osteoblasts involved in bone remodeling.

All of the above described applications may apply to veterinary medicine.

In another embodiment, the invention provides methods and compositions for stimulating the proliferation, infiltration, and/or activation of DR3 receptor expressing cells (e.g. T-cells), comprising, or alternatively consisting of, administering to an animal in which such proliferation, infiltration, and/or activation of DR3 receptor expressing cells is desired, agonistic antibody or agonistic antibody compositions of the invention (e.g., antibody fragments and variants, antibody mixtures, antibody multimers, fusion proteins of the invention, and antibodies in combination with other therapeutic compounds such as antiviral agents) in an amount effective to stimulate proliferation, infiltration, and/or activation of DR3 receptor expressing cells.

In one aspect, the present invention is directed to a method for inhibiting proliferation, infiltration, and/or activation induced by a DR3 receptor ligand (e.g., TL1A), which involves administering to a cell which expresses a DR3 receptor polypeptide an effective amount of an antibody of the invention, preferably an antagonistic anti-DR3 antibody or fragment thereof, capable of inhibiting or decreasing DR3 receptor mediated signaling. Preferably, DR3 receptor mediated signaling is decreased or inhibited by an antibody of the invention to treat a disease wherein decreased proliferation, infiltration, or decreased cytokine and adhesion molecule expression is exhibited.

In a further aspect, the present invention is directed to a method for stimulating proliferation, infiltration and/or activation induced by a DR3 receptor ligand (e.g., TL1A), which involves administering to a cell which expresses a DR3 receptor polypeptide, an effective amount of an antibody of the invention, preferably an agonistic anti-DR3 antibody or fragment thereof, capable of increasing DR3 receptor mediated signaling. Preferably, DR3 receptor mediated signaling is increased to treat a disease wherein increased proliferation, infiltration, or activation is exhibited.

Further, antibodies of the present invention (including molecules comprising, or alternatively consisting of, antibody fragments or variants thereof) which inhibit DR3 receptor-mediated biological activities (e.g., the induction of proliferation in DR3 receptor expressing cells) can be administered to an animal to treat, prevent or ameliorate a disease or disorder described herein, particularly cancers and other hyperproliferative disorders, T-cell activation, autoimmune disorders, as well as graft rejection and graft-versus-host disease. These antibodies may inhibit either all or a subset of the biological activities of DR3 receptor, for example, by inducing a conformational change in DR3 receptor. In a specific embodiment, an antibody of the present invention that decreases DR3 receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to DR3 receptor activity in absence of the antibody is administered to an animal to treat, prevent or ameliorate a disease or disorder. In another embodiment, a combination of antibodies, a combination of antibody fragments, a combination of antibody variants, or a combination of antibodies, antibody fragments and/or antibody variants that decrease DR3 receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to DR3 receptor activity in absence of the said antibodies or antibody fragments and/or antibody variants is administered to an animal to treat, prevent or ameliorate a disease or disorder.

Further, antibodies of the present invention (including molecules comprising, or alternatively consisting of, antibody fragments or variants thereof) which inhibit DR3 receptor-mediated biological activities (e.g., the induction of profileration in DR3 receptor expressing cells) can be administered to an animal to treat, prevent or ameliorate a disease or disorder associated with aberrant DR3 receptor expression, excessive DR3 receptor function, aberrant DR3 receptor ligand expression, or excessive DR3 receptor ligand function. These antibodies may inhibit either all or a subset of the biological activities of DR3 receptor, for example, by inducing a conformational change in DR3 receptor. In a specific embodiment, an antibody of the present invention that decreases DR3 receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to DR3 receptor activity in absence of the antibody is administered to an animal to treat, prevent or ameliorate a disease or disorder associated with aberrant DR3 receptor expression, excessive DR3 receptor function, aberrant DR3 receptor ligand expression, or excessive DR3 receptor ligand function. In another embodiment, a combination of antibodies, a combination of antibody fragments, a combination of antibody variants, or a combination of antibodies, antibody fragments and/or antibody variants that increase DR3 receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to DR3 receptor activity in absence of the said antibodies or antibody fragments and/or antibody variants is administered to an animal to treat, prevent or ameliorate a disease or disorder associated with aberrant DR3 receptor expression or excessive DR3 receptor function or aberrant DR3 receptor ligand expression or excessive DR3 receptor ligand function.

Antibodies of the present invention (including molecules comprising, or alternatively consisting of, antibody fragments or variants thereof) that function as agonists or antagonists of a DR3 receptor, preferably of DR3 receptor signal transduction, can be administered to an animal to treat, prevent or ameliorate a disease or disorder associated with aberrant DR3 receptor expression, excessive DR3 receptor function, aberrant DR3 receptor ligand expression, or excessive DR3 receptor ligand function. For example, antibodies of the invention which mimic the action of DR3 binding to the DR3 receptor, in full or in part, (e.g. antibodies that act as DR3 receptor agonists), may be administered to an animal to treat, prevent or ameliorate a disease or disorder associated with aberrant DR3 receptor expression, excessive DR3 receptor function, aberrant DR3 receptor ligand expression, or excessive DR3 receptor ligand function. As an alternative example, antibodies of the invention which disrupt or prevent the interaction between DR3 receptor and its ligand or stimulate or increase signal transduction through one or more DR3 receptors, may be administered to an animal to treat, prevent or ameliorate a disease or disorder associated with aberrant DR3 receptor expression, excessive DR3 receptor function, aberrant DR3 receptor ligand expression, or excessive DR3 receptor ligand function. Antibodies of the invention which do not prevent a DR3 receptor from binding its ligand but stimulate or upregulate DR3 receptor signal transduction can be administered to an animal to treat, prevent or ameliorate a disease or disorder associated with aberrant DR3 receptor expression, excessive DR3 receptor function, aberrant DR3 receptor ligand expression, or excessive DR3 receptor ligand function. The ability of an antibody of the invention to enhance, inhibit, upregulate or downregulate DR3 receptor signal transduction may be determined by techniques described herein or otherwise known in the art. For example, DR3-induced receptor activation and the activation of signaling molecules can be determined by detecting the association of adaptor proteins such as TRADD with the DR3 receptors, by immunoprecipitation followed by western blot analysis.

Further, antibodies of the present invention (including molecules comprising, or alternatively consisting of, antibody fragments or variants thereof) which inhibit DR3 receptor-mediated biological activities (e.g., the induction of proliferation in DR3 receptor expressing cells) can be administered to an animal to treat, prevent or ameliorate a disease or disorder associated with aberrant DR3 receptor expression, excessive DR3 receptor function, aberrant DR3 receptor ligand expression, or excessive DR3 receptor ligand function. These antibodies may inhibit or deactivate either all or a subset of the biological activities of DR3 receptor, for example, by inducing a conformational change in DR3 receptor. In a specific embodiment, an antibody of the present invention that decreases DR3 receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to DR3 receptor activity in absence of the antibody is administered to an animal to treat, prevent or ameliorate a disease or disorder associated with aberrant DR3 receptor expression, excessive DR3 receptor function, aberrant DR3 receptor ligand expression, or excessive DR3 receptor ligand function. In another embodiment, a combination of antibodies, a combination of antibody fragments, a combination of antibody variants, or a combination of antibodies, antibody fragments and/or antibody variants that increase DR3 receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to DR3 receptor activity in absence of the said antibodies or antibody fragments and/or antibody variants is administered to an animal to treat, prevent or ameliorate a disease or disorder associated with aberrant DR3 receptor expression or lack of DR3 receptor function or aberrant DR3 receptor ligand expression or lack of DR3 receptor ligand function.

In a specific embodiment, an antibody of the present invention (including molecules comprising, or alternatively consisting of, antibody fragments or variants thereof) that inhibits or downregulates, in full or in part, DR3 receptor activity (e.g., stimulation of proliferation) by at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to DR3 receptor activity in absence of the antibody is administered to an animal to treat, prevent or ameliorate a disease or disorder associated with aberrant DR3 receptor expression, lack of DR3 receptor function, aberrant DR3 receptor ligand expression, or lack of DR3 receptor ligand function. In another embodiment, a combination of antibodies, a combination of antibody fragments, a combination of antibody variants, or a combination of antibodies, antibody fragments, and/or variants that stimulate or upregulate DR3 receptor activity by at least 95%, at least 90%, at least 85%, at least 80%, at least 75%, at least 70%, at least 65%, at least 60%, at least 55%, at least 50%, at least 45%, at least 40%, at least 45%, at least 35%, at least 30%, at least 25%, at least 20%, or at least 10% relative to DR3 receptor activity in absence of said antibodies, antibody fragments, and/or antibody variants are administered to an animal to treat, prevent or ameliorate a disease or disorder associated with aberrant DR3 receptor expression, lack of DR3 receptor function, aberrant DR3 receptor ligand expression, or lack of DR3 receptor ligand function.

Therapeutic or pharmaceutical compositions of the invention, may also be administered to treat, prevent, or ameliorate organ rejection or graft-versus-host disease (GVHD) and/or conditions associated therewith. Organ rejection occurs by host immune cell destruction of the transplanted tissue through an immune response. Similarly, an immune response is also involved in GVHD, but, in this case, the foreign transplanted immune cells destroy the host tissues. Thus, the administration of antibodies of the invention, (e.g., antagonistic anti-DR3 receptor antibodies of the invention), may be an effective therapy in preventing organ rejection or GVHD.

In another embodiments, antibodies and antibody compositions of the present invention are used to treat, prevent, or ameliorate diseases associated with increased apoptosis including, but not limited to, neurodegenerative disorders (such as Alzheimer's disease, Parkinson's disease, Amyotrophic lateral sclerosis, Retinitis pigmentosa, Cerebellar degeneration), brain tumor or prion associated disease); autoimmune disorders (such as, multiple sclerosis, Rheumatoid Arthritis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

In additional embodiments, antibodies of the present invention, particularly agonistic anti-DR3 receptor antibodies, are administered in combination with other stimulators of T cell differentiation.

Suitable agents, which also block binding of DR3 ligand (e.g., TL1A) to a DR3 receptor that may be administered with the antibodies of the present invention include, but are not limited to, soluble DR3 receptor polypeptides; multimeric forms of soluble DR3 receptor polypeptides; and DR3 receptor antibodies that bind the DR3 receptor without transducing the biological signal that results in proliferation and/or differentiation, anti-DR3 antibodies that block binding of DR3 to one or more DR3 receptors, and muteins of DR3 that bind DR3 receptors but do not transduce the biological signal that results in proliferation and/or differentiation.

Antibodies and antibody compositions of the invention may be useful for treating inflammatory diseases, such as rheumatoid arthritis, osteoarthritis, psoriasis, septicemia, and inflammatory bowel disease.

In addition, due to lymphoblast expression of DR3 receptor polypeptides, antibodies and antibody compositions of the invention may be used to treat this form of cancer. Further, antibodies and antibody compositions of the invention may be used to treat various chronic and acute forms of inflammation such as rheumatoid arthritis, osteoarthritis, psoriasis, septicemia, and inflammatory bowel disease.

Antibodies and antibody compositions of the invention are useful in the diagnosis and treatment or prevention of a wide range of diseases and/or conditions. Such diseases and conditions include, but are not limited to, cancer (e.g., immune cell related cancers, breast cancer, prostate cancer, ovarian cancer, follicular lymphoma, cancer associated with mutation or alteration of p53, brain tumor, bladder cancer, uterocervical cancer, colon cancer, colorectal cancer, non-small cell carcinoma of the lung, small cell carcinoma of the lung, stomach cancer, etc.), lymphoproliferative disorders (e.g., lymphadenopathy), atherosclerosis, pain, cardiovascular disorders (e.g., neovascularization, hypovascularization or reduced circulation (e.g., ischemic disease (e.g., myocardial infarction, stroke, etc.))), allergy, inflammation, neurodegenerative disease (e.g., Alzheimer's disease, Parkinson's disease, amyotrophic lateral sclerosis, pigmentary retinitis, cerebellar degeneration, etc.), graft rejection (acute and chronic), graft vs. host disease, diseases due to osteomyelodysplasia (e.g., aplastic anemia, etc.), joint tissue destruction in rheumatism, liver disease (e.g., acute and chronic hepatitis, liver injury, and cirrhosis), autoimmune disease (e.g., multiple sclerosis, rheumatoid arthritis, systemic lupus erythematosus, autoimmune lymphoproliferative syndrome (ALPS), immune complex glomerulonephritis, autoimmune diabetes, autoimmune thrombocytopenic purpura, Grave's disease, Hashimoto's thyroiditis, etc.), cardiomyopathy (e.g., dilated cardiomyopathy), diabetes, diabetic complications (e.g., diabetic nephropathy, diabetic neuropathy, diabetic retinopathy), influenza, asthma, psoriasis, glomerulonephritis, septic shock, osteoporosis, and ulcerative colitis.

More generally, antibodies and antibody compositions of the invention are useful in regulating (i.e., elevating or reducing) immune response. For example, antibodies and antibody compositions of the invention are useful as immunosuppressive agents, for example in the treatment or prevention of autoimmune disorders. In specific embodiments, antibodies and antibody compositions of the invention are used to treat or prevent chronic inflammatory, allergic or autoimmune conditions, such as those described herein or are otherwise known in the art.

### Therapeutic/Prophylactic Compositions and Administration

The invention provides methods of treatment, inhibition and prophylaxis by administration to a subject of an effective amount of antibody (or fragment or variant thereof) or pharmaceutical composition of the invention, preferably an antibody of the invention. In a preferred aspect, an antibody or fragment or variant thereof is substantially purified (i.e., substantially free from substances that limit its effect or produce undesired side-effects). The subject is preferably an animal, including but not limited to, animals such as cows, pigs, horses, chickens, cats, dogs, etc., and is preferably a mammal, and most preferably a human.

### Formulation

The invention also provides methods of treatment and/or prevention of diseases or disorders (such as, for example, any one or more of the diseases or disorders disclosed herein) by administration to a subject of an effective amount of a Therapeutic. By therapeutic is meant antibodies of the invention (including fragments and variants) in combination with a pharmaceutically acceptable carrier type (e.g., a sterile carrier).

Formulations and methods of administration that can be employed when the compound comprises a nucleic acid or an immunoglobulin are described above; additional appropriate formulations and routes of administration can be selected from among those described herein below.

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Various delivery systems are known and can be used to administer antibody or fragment or variant thereof of the invention, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

Therapeutics can be are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics include suitable polymeric materials (such as, for example, semi-permeable polymer matrices in the form of shaped articles, e.g., films, or microcapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly(2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

Sustained-release Therapeutics also include liposomally entrapped Therapeutics of the invention (see generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing the Therapeutic are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The Therapeutic is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

Combination Therapies with Anti-DR3 Receptor Antibodies

The Therapeutics of the invention may be administered alone or in combination with other therapeutic agents. Therapeutic agents that may be administered in combination with the Therapeutics of the invention, include but are not limited to, steroidal and non-steroidal anti-inflammatory agents, immunosupressive agents, immunomodulatory agents, chemotherapeutic agents, somatostatic agents, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In a preferred embodiment, the Therapeutics of the invention are administered alone or in combination with an anti-inflammatory agent. Anti-inflammatory agents that may be administered with the Therapeutics of the invention include, but are not limited to, corticosteroids (e.g. betamethasone, budesonide, cortisone, dexamethasone, hydrocortisone, methylprednisolone, prednisolone, prednisone, and triamcinolone), nonsteroidal anti-inflammatory drugs (e.g., balsalazide, celecoxib, diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, olsalazine, oxaprozin, phenylbutazone, piroxicam, salsalate, sulindac, tenoxicam, tiaprofenic acid, and tolmetin.), as well as acetaminophen, antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives (e.g. sulfasalazone, and mesalamine), thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Conventional nonspecific immunosuppressive agents, that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, natalizumab, and other immunosuppressive agents that act by suppressing the function of responding T cells.

In specific embodiments, Therapeutics of the invention are administered in combination with immunosuppressants. Immunosuppressants preparations that may be administered with the Therapeutics of the invention include, but are not limited to, ORTHOCLONE™ (OKT3), SANDIMMUNE™/NEORAL™/SANGDYA™ (cyclosporin), PROGRAF™ (tacrolimus), CELLCEPT™ (mycophenolate), Azathioprine, glucorticosteroids, AVONEX™ (interferon-beta 1A), and RAPAMUNE™ (sirolimus). In a specific embodiment, immunosuppressants may be used to prevent rejection of organ or bone marrow transplantation.

In another embodiment, compostions of the invention are administered in combination with a chemotherapeutic agent. Chemotherapeutic agents that may be administered with the Therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, epinephrine, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, Therapeutics of the invention are administered alone or in combination with a bisphosphonate. Bisphosphonates that may be administered with the Therapeutics of the invention include, but are not limited to, alendronate (FOSAMAX™), risedronate (ACTONEL™), ibandronate (BONIVA™), zoledronic acid (RECLAST™) and etidronate (DIDRONEL™).

In an additional embodiment, Therapeutics of the invention are administered alone or in combination with hormones and other hormone-like medications. Hormones and other hormone-like medications that may be administered with the Therapeutics of the invention include, but are not limited to, natural and synthetic forms of estrogen, testosterone, androgens, calcitonin (CALCIMAR™, MIACALCIN™, or FORTICAL™), and parathyroid hormone (teriparatide, FORTEO™).

In an additional embodiment, Therapeutics of the invention are administered alone or in combination with selective estrogen receptor modulators (SERMs). SERMs that may be administered with therapeutics of the invention include, but are not limited to, raloxifene (EVISTA™), tamoxifen (NOLVADEX™), and toremifene (FARESTON™).

In another embodiment, compostions of the invention are administered in combination with an anti-diarrheal agent. Anti-diarreal agents that may be administered with the Therapeutics of the invention include, but are not limited to, loperamide (e.g. IMODIUM™), and bismuth subsalicylate.

In another embodiment, compositions of the invention are administered in combination with a TNF-alpha antagonist. TNF antagonists that may be administered with the Therapeutics of the invention include, but are not limited to, infliximab (REMICADE™), adalimumab (HUMIRA™), certolizumab pegol (CIMZIA™), golimumab (SIMPONI™), etanercept (ENBREL™), xanthine deriviatives (e.g. pentoxifylline) and bupropion (WELLBUTRIN™, ZYBAN™).

In another embodiment, compositions of the invention are administered in combination with a RANK ligand (RANK-L) antagonist. RANK-L antagonists that may be administered with the Therapeutics of the invention include, but are not limited to, denosumab (PROLIA™).

In another embodiment, compositions of the invention are administered in combination with an anticholinergic agent. Anticholinergic agents that may be administered with the Therapeutics of the invention include, but are not limited to, clidimium/chlordiazepoxide (LIBRAX™), cyproheptadine, dicyclomine (BENTYL™), glycopyrrolate (ROBINUL™), (LEVSIN™), hyoscyamine sulfate (LEVBID™, SYMAX™, DUOTAB™), and ipratropium.

In an additional embodiment, Therapeutics of the invention are administered alone or in combination with a bronchiodilator. Bronchodilator agents that may be administered with the Therapeutics of the invention include, but are not limited to, fromoterol, levalbuterol, metaproterenol, pirbuterol, tertbtaline, and salmeterol.

In an additional embodiment, Therapeutics of the invention are administered alone or in combination with a leukotrine antagonist. Leukotrine antagonist agents that may be administered with the Therapeutics of the invention include, but are not limited to, montelukast (SINGULAIR™) and zafirlukast (ACCOLTE™).

In an additional embodiment, Therapeutics of the invention are administered alone or in combination with a muscle relaxant. Muscle relaxant agents that may be administered with the Therapeutics of the invention include, but are not limited to, baclofen (LIORESAL™), tizanidine (ZANAFLEX™), and dantrolene (DANTRIUM™).

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR™, IVEEGAM™, SANDOGLOBULIN™, GAMMAGARD S/D™, and GAMIMUNE™. In a specific embodiment, Therapeutics of the invention are administered in combination with intravenous immune globulin preparations in transplantation therapy (e.g., bone marrow transplant).

In a specific embodiment, Therapeutics of the invention are administered in combination with CHOP (cyclophosphamide, doxorubicin, vincristine, and prednisone) or any combination of the components of CHOP. In another embodiment, Therapeutics of the invention are administered in combination with Rituximab. In a further embodiment, Therapeutics of the invention are administered with Rituximab and CHOP, or Rituximab and any combination of the components of CHOP.

In an additional embodiment, the Therapeutics of the invention are administered in combination with cytokines Cytokines that may be administered with the Therapeutics of the invention include, but are not limited to, IL2, IL3, IL4, IL5, IL6, IL7, IL10, IL12, IL13, IL15, anti-CD40, CD40L, IFN-γ and TNF-α. In another embodiment, Therapeutics of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21.

The invention also encompasses combining the antibodies of the invention with other proposed or conventional hematopoietic therapies. Thus, for example, the antibodies of the invention can be combined with compounds that singly exhibit erythropoietic stimulatory effects, such as erythropoietin, testosterone, progenitor cell stimulators, insulin-like growth factor, prostaglandins, serotonin, cyclic AMP, prolactin, and triiodothyzonine. Also encompassed are combinations of the antibody and antibody compositions of the invention with compounds generally used to treat aplastic anemia, such as, for example, methenolene, stanozolol, and nandrolone; to treat iron-deficiency anemia, such as, for example, iron preparations; to treat malignant anemia, such as, for example, vitamin B₁₂ and/or folic acid; and to treat hemolytic anemia, such as, for example, adrenocortical steroids, e.g., corticoids. See e.g., Resegotti et al., Panminerva Medica, 23:243-248 (1981); Kurtz, FEBS Letters, 14a:105-108 (1982); McGonigle et al., Kidney Int., 25:437-444 (1984); and Pavlovic-Kantera, Expt. Hematol., 8(supp. 8) 283-291 (1980), the contents of each of which are hereby incorporated by reference in their entireties.

Compounds that enhance the effects of or synergize with erythropoietin are also useful as adjuvants herein, and include but are not limited to, adrenergic agonists, thyroid hormones, androgens, hepatic erythropoietic factors, erythrotropins, and erythrogenins, See for e.g., Dunn, “Current Concepts in Erythropoiesis”, John Wiley and Sons (Chichester, England, 1983); Kalmani, Kidney Int., 22:383-391 (1982); Shahidi, New Eng. J. Med., 289:72-80 (1973); Urabe et al., J. Exp. Med., 149:1314-1325 (1979); Billat et al., Expt. Hematol., 10:135-140 (1982); Naughton et al., Acta Haemat, 69:171-179 (1983); Cognote et al. in abstract 364, Proceedings 7th Intl. Cong. of Endocrinology (Quebec City, Quebec, Jul. 1-7, 1984); and Rothman et al., 1982, J. Surg. Oncol., 20:105-108 (1982). Methods for stimulating hematopoiesis comprise administering a hematopoietically effective amount (i.e., an amount which effects the formation of blood cells) of a pharmaceutical composition containing polynucleotides and/or polypeptides of the invention (and/or agonists or antagonists thereof) to a patient. The antibody of the invention is administered to the patient by any suitable technique, including but not limited to, parenteral, sublingual, topical, intrapulmonary and intranasal, and those techniques further discussed herein. The pharmaceutical composition optionally contains one or more members of the group consisting of erythropoietin, testosterone, progenitor cell stimulators, insulin-like growth factor, prostaglandins, serotonin, cyclic AMP, prolactin, triiodothyzonine, methenolene, stanozolol, and nandrolone, iron preparations, vitamin B₁₂, folic acid and/or adrenocortical steroids.

In an additional embodiment, the antibody and antibody compositions of the invention are administered in combination with hematopoietic growth factors. Hematopoietic growth factors that may be administered with the antibody and antibody compositions of the invention include, but are not limited to, LEUKINE™ (SARGRAMOSTIM™) and NEUPOGEN™ (FILGRASTIM™).

In one embodiment, the antibody and antibody compositions of the invention are administered in combination with one or more chemokines. In specific embodiments, the antibody and antibody compositions of the invention are administered in combination with an α(C×C) chemokine selected from the group consisting of gamma-interferon inducible protein-10 (γIP-10), interleukin-8 (IL-8), platelet factor-4 (PF4), neutrophil activating protein (NAP-2), GRO-α, GRO-β, GRO-γ, neutrophil-activating peptide (ENA-78), granulocyte chemoattractant protein-2 (GCP-2), and stromal cell-derived factor-1 (SDF-1, or pre-B cell stimulatory factor (PBSF)); and/or a β(CC) chemokine selected from the group consisting of: RANTES (regulated on activation, normal T expressed and secreted), macrophage inflammatory protein-1 alpha (MIP-1α), macrophage inflammatory protein-1 beta (MIP-β), monocyte chemotactic protein-1 (MCP-1), monocyte chemotactic protein-2 (MCP-2), monocyte chemotactic protein-3 (MCP-3), monocyte chemotactic protein-4 (MCP-4) macrophage inflammatory protein-1 gamma (MIP-1γ), macrophage inflammatory protein-3 alpha (MIP-3α), macrophage inflammatory protein-3 beta (MIP-3β), macrophage inflammatory protein-4 (MIP-4/DC-CK-1/PARC), eotaxin, Exodus, and I-309; and/or the γ(C) chemokine, lymphotactin.

In another embodiment, the antibody and antibody compositions of the invention are administered with chemokine beta-8, chemokine beta-1, and/or macrophage inflammatory protein-4. In a preferred embodiment, the antibody and antibody compositions of the invention are administered with chemokine beta-8.

In an additional embodiment, the antibody and antibody compositions of the invention are administered in combination with an IL-4 antagonist. IL-4 antagonists that may be administered with the antibody and antibody compositions of the invention include, but are not limited to: soluble IL-4 receptor polypeptides, multimeric forms of soluble IL-4 receptor polypeptides; anti-IL-4 receptor antibodies that bind the IL-4 receptor without transducing the biological signal elicited by IL-4, anti-IL-4 antibodies that block binding of IL-4 to one or more IL-4 receptors, and muteins of IL-4 that bind IL-4 receptors but do not transduce the biological signal elicited by IL-4. Preferably, the antibodies employed according to this method are monoclonal antibodies (including antibody fragments, such as, for example, those described herein).

In an additional embodiment, the Therapeutics of the invention are administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In another embodiment, the Therapeutics of the invention are administered in combination with members of the TNF family. TNF, TNF-related or TNF-like molecules that may be administered with the Therapeutics of the invention include, but are not limited to, soluble forms of TNF-α, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892), TR10 (International Publication No. WO 98/54202), 312C2 (International Publication No. WO 98/06842), and TR12, and soluble forms CD154, CD70, and CD153.

In additional embodiments, the Therapeutics of the invention are administered in combination with other therapeutic or prophylactic regimens, such as, for example, radiation therapy.

### Demonstration of Therapeutic or Prophylactic Utility of a Composition

The compounds of the invention are preferably tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a patient tissue sample is grown in culture, and exposed to or otherwise administered an antibody or composition of the present invention, and the effect of such an antibody or composition of the present invention upon the tissue sample is observed. In various specific embodiments, in vitro assays can be carried out with representative cells of cell types involved in a patient's disorder, to determine if an antibody or composition of the present invention has a desired effect upon such cell types. Preferably, the antibodies or compositions of the invention are also tested in in vitro assays and animal model systems prior to administration to humans.

Antibodies or compositions of the present invention for use in therapy can be tested for their toxicity in suitable animal model systems, including but not limited to rats, mice, chicken, cows, monkeys, and rabbits. For in vivo testing of an antibody or composition's toxicity any animal model system known in the art may be used.

Antibodies or compositions of the invention can be tested for their ability to reduce tumor formation in in vitro, ex vivo and in vivo assays. Antibodies or compositions of the invention can also be tested for their ability to inhibit cell proliferation or activation in in vitro and in vivo assays. Antibodies or compositions of the invention can also be tested for their ability to alleviate of one or more symptoms associated with cancer, an immune disorder (e.g., an inflammatory disease), or a neurological disorder. Further, antibodies or compositions of the invention can be tested for their ability to increase the survival period of animals suffering from disease or disorder, including cancer, an immune disorder or an infectious disease. Techniques known to those of skill in the art can be used to analyze the function of the antibodies or compositions of the invention in vivo.

Antibodies or compositions of the invention can be tested for their ability to modulate the biological activity of immune cells by contacting immune cells, preferably human immune cells (e.g., T-cells, B-cells, and Natural Killer cells), with an antibody or composition of the invention or a control compound and determining the ability of the antibody or composition of the invention to modulate (i.e, increase or decrease) the biological activity of immune cells. The ability of an antibody or composition of the invention to modulate the biological activity of immune cells can be assessed by detecting the expression of antigens, detecting the proliferation of immune cells (i.e., T-cell proliferation), detecting the activation of signaling molecules, detecting the effector function of immune cells, or detecting the differentiation of immune cells. Techniques known to those of skill in the art can be used for measuring these activities. For example, cellular proliferation can be assayed by ³H-thymidine incorporation assays and trypan blue cell counts. Antigen expression can be assayed, for example, by immunoassays including, but not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs). In a preferred embodiment, the ability of an antibody or composition of the invention to inhibit or induce T-cell proliferation is measured. In another preferred embodiment, the ability of an antibody or composition of the invention to modulate cytokine release is measured.

### Panels/Mixtures

The present invention also provides for mixtures of antibodies (including scFvs and other molecules comprising, or alternatively consisting of, antibody fragments or variants thereof) that specifically bind to DR3 receptor or a fragment or variant thereof, wherein the mixture has at least one, two, three, four, five or more different antibodies of the invention. In specific embodiments, the invention provides mixtures of at least 2, preferably at least 4, at least 6, at least 8, at least 10, at least 12, at least 15, at least 20, or at least 25 different antibodies that specifically bind to DR3 receptor or fragments or variants thereof, wherein at least 1, at least 2, at least 4, at least 6, or at least 10, antibodies of the mixture is an antibody of the invention. In a specific embodiment, each antibody of the mixture is an antibody of the invention.

The present invention also provides for panels of antibodies (including scFvs and other molecules comprising, or alternatively consisting of, antibody fragments or variants thereof) that specifically bind to DR3 receptor or a fragment or variant thereof, wherein the panel has at least one, two, three, four, five or more different antibodies of the invention. In specific embodiments, the invention provides for panels of antibodies that have different affinities for DR3 receptor, different specificities for DR3 receptor, or different dissociation rates. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000, antibodies. Panels of antibodies can be used, for example, in 96 well plates for assays such as ELISAs.

The present invention further provides for compositions comprising, one or more antibodies (including molecules comprising, or alternatively consisting of antibody fragments or variants of the invention). In one embodiment, a composition of the present invention comprises, one, two, three, four, five, or more antibodies that comprise or alternatively consist of, a polypeptide having an amino acid sequence of any one or more of the VH domains referred to in Table 1 or a variant thereof. In another embodiment, a composition of the present invention comprises, one, two, three, four, five, or more antibodies that comprise, or alternatively consist of, a polypeptide having an amino acid sequence of any one or more of the VH CDR1s of a heavy chain referred to in Table 1 or a variant thereof. In another embodiment, a composition of the present invention comprises, one, two, three, four, five or more antibodies that comprise, or alternatively consist of, a polypeptide having an amino acid sequence of any one or more of the VH CDR2s of a heavy chain referred to in Table 1, or a variant thereof. In a preferred embodiment, a composition of the present invention comprises, one, two, three, four, five, or more antibodies that comprise, or alternatively consist of, a polypeptide having an amino acid sequence of any one or more of the VH CDR3s as of a heavy chain referred to in Table 1, or a variant thereof.

Other embodiments of the present invention providing for compositions comprising, one or more antibodies (including molecules comprising, or alternatively consisting of antibody fragments or variants of the invention) are listed below. In another embodiment, a composition of the present invention comprises, one, two, three, four, five, or more antibodies that comprise, or alternative consist of, a polypeptide having an amino acid sequence of any one or more of the VL domains of a light chain referred to in Table 1, or a variant thereof. In another embodiment, a composition of the present invention comprises, one, two, three, four, five, or more antibodies that comprise, or alternatively consist of, a polypeptide having an amino acid sequence of any one or more of the VL CDR1s of a light chain referred to in Table 1, or a variant thereof. In another embodiment, a composition of the present invention comprises, one, two, three, four, five, or more antibodies that comprise, or alternatively consist of, a polypeptide having an amino acid sequence of any one or more of the VL CDR2s of a light chain referred to in Table 1, or a variant thereof. In a preferred embodiment, a composition of the present invention comprises, one, two, three, four, five, or more antibodies that comprise, or alternatively consist of, a polypeptide having an amino acid sequence of any one or more of the VL CDR3s of a light chain referred to in Table 1, or a variant thereof.

### Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an antibody of the invention, preferably a purified antibody, in one or more containers. In an alternative embodiment, a kit comprises an antibody fragment that specifically binds to DR3 receptor polypeptides or fragments or variants thereof. In a specific embodiment, the kits of the present invention contain a substantially isolated DR3 receptor polypeptide or fragment or variant thereof as a control. Preferably, the kits of the present invention further comprise a control antibody which does not react with any, some or all DR3 receptors. In another specific embodiment, the kits of the present invention contain a means for detecting the binding of an antibody to DR3 receptor polypeptides (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized DR3 receptor. The DR3 receptor provided in the kit may also be attached to a solid support. In a more specific embodiment the detecting means of the above-described kit includes a solid support to which DR3 receptor is attached. Such a kit may also include a non-attached reporter-labeled anti-human antibody. In this embodiment, binding of the antibody to DR3 receptor can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with a DR3 receptor, and means for detecting the binding of DR3 receptor polypeptides to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having surface-bound DR3 receptors obtained by the methods of the present invention. After DR3 receptor polypeptides bind to a specific antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-DR3 receptor antibody on the solid support. Typically, the reporter is an enzyme which is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant DR3 receptor, and a reporter-labeled anti-human antibody for detecting surface-bound anti-DR3 receptor antibody.

### EXAMPLES

### Example 1

### Generation of a Mouse Anti-Human DR3 Monoclonal Antibody

A mouse monoclonal antibody that specifically binds the extracellular domain of human DR3 was generated by hybridoma technology. Typical procedures for generating a mouse monoclonal antibody are well-known in the art and are described by Kohler and Millstein (1975) and others. Briefly, mice are immunized with the target antigen to induce the generation of antibodies. After 4 to 12 weeks of immunization, the splenocytes from the immunized animals are isolated and fused with a suitable myeloma cell line. Any suitable myeloma cell line known in the art may be employed in accordance with the present invention. After fusion, the resulting hybridoma cells are seeded to a density of one cell per well and maintained in selective HAT (hypoxanthine-aminopterin-thymidine) medium. Seven days post-fusion, the cells are re-fed with HT (hypoxanthine-thymidine) medium and then cloned by limiting dilution as described by Wands et al. (Gastroenterology 80:225-232 (1981)). The hybridoma cells obtained through such selection are then assayed via techniques such as ELISA and FACS to identify which clones secrete antibodies capable of selectively binding the antigen target.

To generate a DR3 monoclonal antibody, mice were immunized with a fragment of the human DR3 polypeptide fused with a human Fc resulting in a hybridoma capable of producing anti-DR3 antibodies. Preferred fragments of the human DR3 polypeptide would include the extracellular domain of the polypeptide or a portion thereof. The hybridoma cells were then assayed to identify which clones secrete antibodies capable of selectively binding the extracellular domain of human DR3. The 11H08 clone (mouse isotype IgG1κ) was selected for further testing and humanization of its sequence.

### Example 2

### TL1A Binding Inhibition Assay Using 11H08

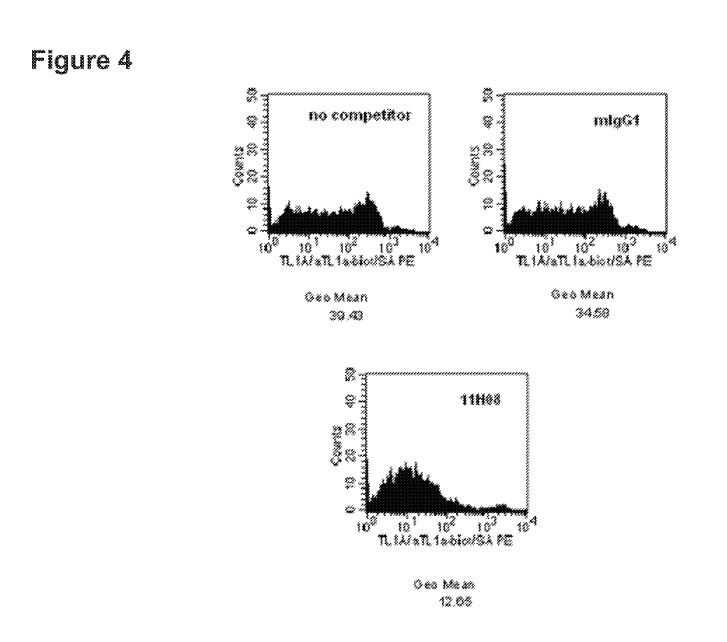

The anti-DR3 monoclonal antibody 11H08 was assayed for its ability to inhibit human TL1A binding to human DR3 using techniques known to those skilled in the art. Briefly, HEK293F cells transiently expressing human DR3 were incubated with 1 μg/mL of human TL1A in the presence of either an unrelated antibody (mIgG1) as an isotype control that was known not to bind TL1A or DR3, or an anti-DR3 monoclonal antibody (11H08), for 20 minutes at room temperature. Binding was detected using an anti-TL1A-biotin polyclonal antibody followed by Streptavidin PE. The ability of the anti-DR3 monoclonal antibody to inhibit binding of TL1A to DR3 was measured by flow cytometry. As shown in FIG. 4, addition of the 11H08 monoclonal antibodies inhibited human TL1A binding to the human DR3 receptor.

### Example 3

### Cloning of VH and VL domains

VH and VL domains can be isolated, cloned, subcloned, and sequenced using methods commonly known in the art. For example, VH and VL domains can be isolated and cloned from cell lines expressing a particular antibody using RT-PCR with VH and VL specific primers on cDNA made from the antibody expressing cell lines. Briefly, RNA is isolated from the cell lines and used as a template for RT-PCR designed to amplify the VH and VL domains of the antibodies. Cells are lysed in the TRIzol® reagent (Life Technologies, Rockville, Md.) and extracted with one fifth volume of chloroform. After addition of chloroform, the solution is allowed to incubate at room temperature for 10 minutes, and the centrifuged at 14,000 rpm for 15 minutes at 4° C. in a tabletop centrifuge. The supernatant is collected and RNA is precipitated using an equal volume of isopropanol. Precipitated RNA is pelleted by centrifuging at 14,000 rpm for 15 minutes at 4° C. in a tabletop centrifuge. Following centrifugation, the supernatant is discarded and washed with 75% ethanol. Following washing, the RNA is centrifuged again at 800 rpm for 5 minutes at 4° C. The supernatant is discarded and the pellet allowed to air dry. RNA is the dissolved in DEPC-treated water and heated to 60° C. for 10 minutes. Quantities of RNA can be determined using optical density measurements at 260 nm. cDNA is synthesized from 1.5-2.5 micrograms of RNA using reverse transciptase and random hexamer primers.

cDNA is used as a template for PCR amplification of VH and VL domains. Primers used to amplify murine VH and VL genes are well-known in the art and are commercially available (e.g., EMD Chemicals, Inc., Gibbstown, N.J.). In addition, N-terminal sequencing can also be performed on the antibody to determine the sequence of the VH domain, in order to identify N-terminal primers more specific for the N-terminus of the VH domain for use in PCR. Typically, a PCR reaction makes use of a single 5′ primer and a single 3′ primer. Sometimes, when the amount of available RNA template is limiting, or for greater efficiency, groups of 5′ and/or 3′ primers may be used. For example, sometimes all 5′ primers and all 3′ primers are used in a single PCR reaction. The PCR reaction is carried out in a 50 microliter volume containing 1×PCR buffer, 2 mM of each dNTP, 0.7 units of High Fidelity Taq polymerse, 5′ primer mix, 3′ primer mix and 7.5 microliters of cDNA. The 5′ and 3′ primer mix of both VH and VL can be made by pooling together 22 pmole and 28 pmole, respectively, of each of the individual primers. PCR conditions are: 96° C. for 5 minutes; followed by 25 cycles of 94° C. for 1 minute, 50° C. for 1 minute, and 72° C. for 1 minute; followed by an extension cycle of 72° C. for 10 minutes. After the reaction is completed, sample tubes are stored 4° C. PCR samples are then electrophoresed on a 1.3% agarose gel. DNA bands of the expected sizes (−330 base pairs for VH and VL domains) are cut out of the gel and purified using methods well known in the art. Purified PCR products are ligated into a PCR cloning vector (TA vector from Invitrogen Inc., Carlsbad, Calif.). Individual cloned PCR products are isolated after transfection of E. coli and blue/white color selection. Cloned PCR products are sequenced using methods commonly known in the art.

The VH and VL domains from the antibody expressed by the 11H08 hybridoma (SEQ ID NO:7 and SEQ ID NO:20, respectively) were isolated, cloned, sequenced, and subcloned into an appropriate expression vector using standard techniques known in the art. Novagen® Mouse Ig-Primer Set (Cat. No. 69831; EMD Chemicals, Inc., Gibbstown, N.J.) were used to isolate, clone, and subclone the VH and VL domains of the 11H08 antibody.

### Example 4

### Humanization of the 11H08 Antibody

Although the 11H08 monoclonal antibody was effective in in vitro assays, in order to reduce potential immunogenicity from the mouse monoclonal antibody when administered into a human subject, the sequence of the VH and VL domains of 11H08 were humanized to generate variants that would have reduced immunogenic properties yet retain similar binding properties and potency.

A panel of humanized heavy and light chain variable domains were produced by applying string content optimization (see, e.g., U.S. Pat. No. 7,657,380, issued Feb. 2, 2010, and U.S. Publ. No. 2008-0167449, filed Oct. 31, 2007) to the murine anti-DR3 antibody 11H08 produced in Example 1. String content optimization is a method of humanization based on a metric of antibody hummanness, termed human string content (HSC), which quantifies the “humanness” of a sequence by comparison to the human antibody germline at the level of potential MHC/T-cell epitopes. Two different scores were generated for each sequence using this method, HSC as mentioned above and N9, a count of the number of perfect 9-mer matches. These values were optimized by making amino acid substitutions in the murine antibody using residues from structurally analogous positions within the human antibody germline repertoire.

Humanization of the murine 11H08 heavy chain variable domain (H0) was accomplished by making mutations, typically between 10-19, to generate the humanized sequences (H1-H5.3; SEQ ID NOs:9-19, respectively). Humanization of the murine light chain (L0) was accomplished by making mutations, typically between 15-18, resulting in the humanized sequences (L1-L4; SEQ ID NOs:21-24, respectively). A rare amino acid was identified at amino acid position 95 (Kabat position 91) of the 11H08 heavy chain sequence (H0; SEQ ID NO:7). Usually a tyrosine, there was only one occurrence of an asparagine at this position in the Kabat antibody sequence database (Martin, 1996). This is a structurally important position at the VH/VL interface, and is “masked” during HSC optimization. In addition, because the tyrosine at this position was found in most human sequences, it was expected that changing this residue to a tyrosine would result in a “more human” sequence. Therefore, the asparagine to tyrosine mutation at amino acid position 95 (Kabat position 91) of, for example, SEQ ID NO:8, was included in many of the disclosed humanized variants (H1.1, H2.1, H3.1, H4.2, H5.2, H5.3; SEQ ID NOs:10, 12, 14, 16, 18, 19, respectively).

FIG. 1A shows sequence alignments for murine 11H08 heavy chain H0 (SEQ ID NO:7) and humanized heavy chains H1-H5 (SEQ ID NOs:9, 11, 13, 15, and 17, respectively). FIG. 1B shows sequence alignments for murine 11H08 heavy chain H0 (SEQ ID NO:7) and humanized heavy chains H1.1-H5.3 (SEQ ID NOs:10, 12, 14, 16, 18, and 19, respectively). FIG. 2 shows sequence alignments for murine 11H08 light chain L0 (SEQ ID NO:20) and humanized light chains L1-L4. (SEQ ID NOs:21-24, respectively). In both figures, the CDRs are represented by shading.

### Example 5

### TF-1 Assay Using 11H08

Treatment of TF-1 cells with human TL1A (the ligand for DR3) in the presence of cycloheximide induces cell death (Migone et al., Immunity 16:479-492 (2002)). A TF-1 assay was used to test the ability of 11H08 to block TL1A activity through the DR3 receptor.

### TF-1 Assay Method

A TF-1 assay can be performed according to methods known in the art. Briefly, premyeloid TF-1 cells are maintained in 10% FBS (fetal bovine serum), 1×RPMI, 1% Pen-Strep with Glutamine, and 2 ng/mL GM-CSF. The cells are grown until log phase, washed three times in PBS, and incubated with the antibodies to be tested for one hour. After this pre-incubation, between 50,000 and 100,000 cells are seeded per well. The cells are combined with 10 ng/mL hTL1A, 10 μg/mL cycloheximide (Sigma-Aldrich, cat #C1988-1G) and the antibodies to be tested and then incubated for 24 to 72 hours. Cell viability is measured using Cell Titer Glo (Promega, cat #G7570). This assay allows cell viability to be determined by measuring the luminescent signal which is proportional to the amount of ATP present. The ATP is indicative of metabolically active cells and higher luminescence signals reflect greater cell viability.

### The 11H08 Monoclonal Antibody Acts as an Agonist of TL1A Activity

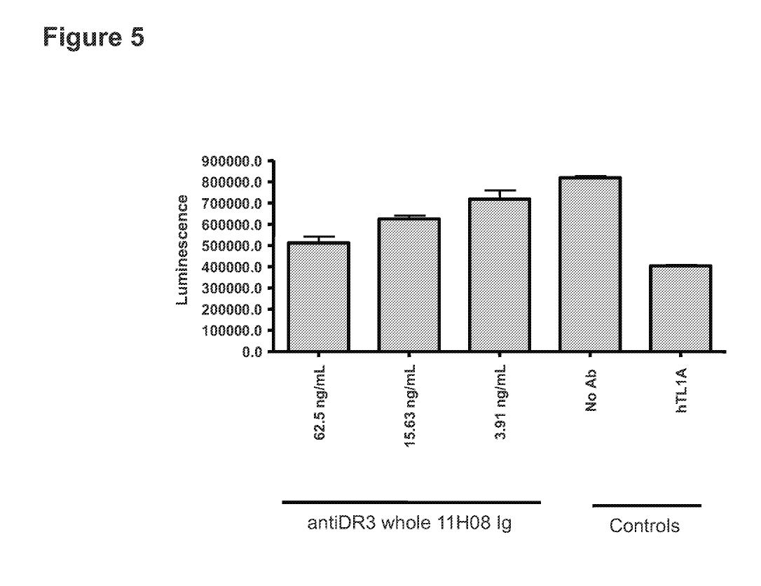

As shown in FIG. 5, various concentrations of 11H08 and the TL1A ligand both induce apoptosis. The addition of TL1A and cycloheximide or the whole 11H08 antibody and cycloheximide to the cells caused a decrease in TF-1 cell viability (measured by luminescence). These results indicate that the full length 11H08 IgG1 antibody activated the DR3 receptor, thereby inducing TL1A-like activity.

While the 11H08 monoclonal antibody would be useful due to its agonistic activities, in order to also generate anti-DR3 molecules that would inhibit the activity of TL1A through the DR3 receptor, monomeric anti-DR3 fragments were generated. In addition, whole IgG molecules generally have a longer in vivo half-life than smaller peptides, such as antibody Fab fragments. To extend the half-life of peptides, various molecules well-known in the art can be conjugated to the peptides. To this end, the monomeric anti-DR3 fragments were fused to serum albumin molecules (e.g., Human Serum Albumin or Mouse Serum Albumin).

### Example 6

### Generation of Constructs for Expression of Specific Anti-Human DR3 Monoclonal Antibody Fragments

### Fab Constructs

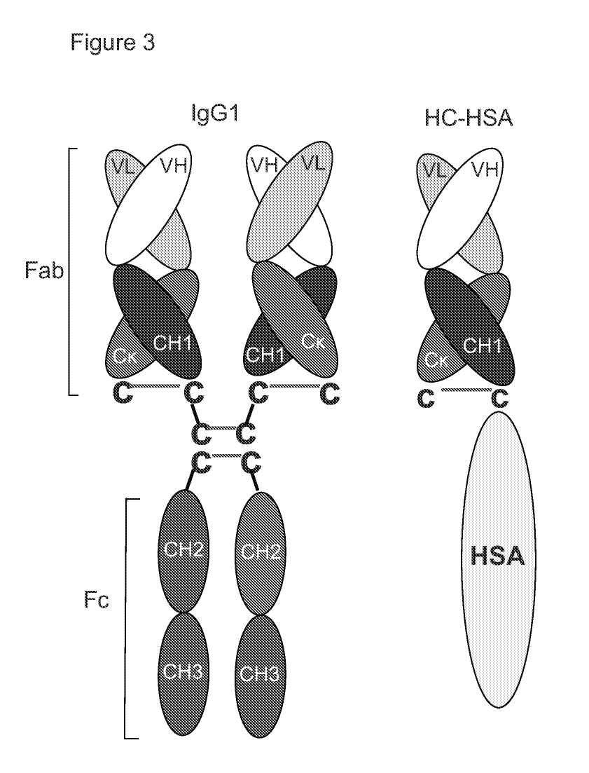

Fab constructs were generated to express specific anti-human DR3 monoclonal antibody fragments depicted by the drawing in FIG. 3. Vectors to express Fab fragments of 11H08, H1L2, and H5L2 were constructed using standard methods in the art. Briefly, the VH and VL domains of 11H08 and variants H1L2 and H5L2 were cloned into appropriate Fab expression vectors in order to express a human anti-DR3-Fab molecule. To facilitate cloning of the humanized VL domains, Kabat position 104 of VL domains L1-L4 (corresponding to residue 108 of SEQ ID NOs:21-24) was changed from a Valine to a Leucine residue. Hereinafter, discussion of humanized expression constructs (e.g., H1L2, H5L2, etc.) contain the Valine-to-Leucine change at Kabat position 104. The VH of each of the constructs was subcloned into a vector whereby the VH nucleotide sequence was fused to the nucleotide sequence of the heavy chain constant region (CH1) from human IgG1 (SEQ ID NO:27) followed by the nucleotide sequence encoding the first 5 amino acids (EPKSC) from the hinge region. The VL of each of the constructs was subcloned into a vector fusing the VL nucleotide sequence with the nucleotide sequence of human light chain constant region kappa (Cκ) (SEQ ID NO:25).

In addition, the VH domain of 11H08 and variants H1L2 and H5L2 were separately cloned into appropriate Fab-HSA expression vectors, as indicated above for the generation of the Fab heavy chain expression vector but where the heavy chain was fused at its C-terminus to the Human Serum Albumin (HSA)-encoding nucleotide sequence (SEQ ID NO:3) in order to express a human anti-DR3-Fab-HSA fusion molecule when expressed along with the light chain expression vector in an appropriate host cell. Further, when cloned heavy and light chains are both expressed in one cell line (from either one or two vectors), they assemble into a complete functional Fab molecule that is secreted into the cell culture medium. The anti-DR3-Fab and anti-DR3-Fab-HSA vectors (H1L2-Fab-HSA and H5L2-Fab-HSA) were transiently expressed in HEK293F cells to produce fusion proteins, which were then purified. Purification can be accomplished using standard techniques in the art including HQ and Protein L chromatography.

For purposes of testing the efficacy of an anti-DR3-Fab molecule in mouse models, a surrogate mouse anti-DR3-Fab-MSA (Mouse Serum Albumin) construct was generated. Mouse DR3-Fc was used to select scFv from human naïve phage display libraries (Cambridge Antibody Technology/MedImmune Cambridge). The specific binders were further screened for their ability to inhibit the binding activity of mouse TL1A to mouse DR3-Fc. The VH and VL domains of the final clone (D06) were sequenced, amplified using PCR, and subcloned into Fab expression vectors, similar to what was described above. For the heavy chain construct, the nucleotide sequence of the VH domain was fused to the mouse IgG2a CH1 (SEQ ID NO:28) followed by the nucleotide sequence encoding MSA (SEQ ID NO:5). In the light chain construct, the nucleotide sequence of the VL domain was fused to a polynucleotide encoding the mouse Cκ (SEQ ID NO:26). When expressed in the same cell, expression of the heavy chain and light chain constructs generated a D06-Fab-MSA molecule. The D06-Fab-MSA vectors were transiently expressed in HEK293F cells to produce fusion proteins, which were then purified. Purification can be accomplished using standard techniques in the art including HQ and Protein L chromatography. This method was used to generate other anti-DR3-Fab-MSA molecules, including but not limited to, 2D9-Fab-MSA and 5D10-Fab-MSA.

scFv Constructs

An 11H08-scFv-HSA construct was generated using standard methods known in the art. Briefly, the VH (SEQ ID NO:7) and VL (SEQ ID NO:20) regions of the 11H08 antibody were engineered to contain a linker sequence between the two domains to generate an scFv molecule. In addition, HSA (SEQ ID NO:3) was fused to the C-terminus of the VH portion of the scFv molecule and the entire construct was subcloned into an appropriate expression vector to produce the 11H08-scFv-HSA molecule. The scFvs expressed by the vector were isolated and confirmed for the ability to bind to human DR3-Fc and inhibit binding of human TL1A to DR3-Fc.

The mouse surrogate D06-scFv construct was generated using standard methods known in the art. Briefly, mouse DR3-Fc was used to select scFvs from human naïve phage display libraries (Cambridge Antibody Technology/Medimmune Cambridge). Those scFvs that specifically bound to mouse DR3-Fc were further screened for their ability to inhibit the binding of murine TL1A to DR3-Fc. The final clone (D06) was subcloned into an appropriate expression vector to produce the D06-scFv molecule.

### Example 7

### Pharmokinetic Analysis of the D06-Fab-MSA Molecule

The D06-Fab-MSA was tested to determine its pharmokinetic profile in BALB/c mice. The construct was compared to a whole immunoglobulin molecule generated using the VH and VL domains from D06 and a Histidine-tagged D06-Fab construct without the mouse serum albumin. The three molecules to be tested were given to the mice at a 1 mg/kg intravenous injection and the presence of these molecules in the mice were then determined. Table 2 shows the serum concentrations of D06-Fab-MSA in the Balb/c mice at various times following the injection.

TABLE 2

| Time following injection | D06-Fab-MSA concentration |
|---|---|
| (hours) | (μg/mL) |
| .083 | 15.8 |
| 1 | 9.0 |
| 6 | 8.7 |
| 24 | 5.1 |
| 48 | 4.0 |
| 72 | 4.6 |
| 144 | 2.1 |
| 240 | 0.4 |

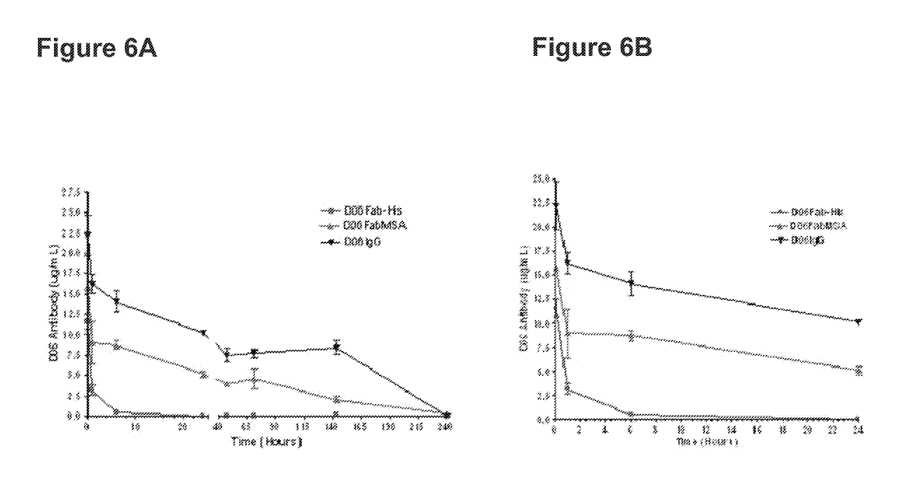

In addition, FIG. 6 shows that the D06-Fab-MSA molecule has a significantly longer half-life in mice than the D06-Fab-His construct without the MSA molecule. Although lower than the whole D06 immunoglobulin molecule, these results suggest that adding a serum albumin protein to an anti-DR3-Fab-molecule would provide therapeutic benefits by allowing the molecule to remain in the host for longer periods of time.

### Example 8

### Mouse CD4⁺ T Cell Proliferation Assay

To test the effects of the surrogate mouse antibody on T cell proliferation, a mouse CD4⁺ T cell proliferation assay was performed using the D06-scFv molecule. A mouse CD4⁺ T Cell Proliferation Assay can be performed using standard techniques in the art. Briefly, murine CD4+ T cells are purified from mouse spleen and lymph nodes using Histopaque 1083 Sigma (Cat #SD10831B) followed by a CD4⁺ negative selection kit (Miltenyi biotec cat #130-090-860). The antibody molecule of interest is pre-incubated with its respective targets for 1 hour. Cells are grown to approximately 50,000 cells per well and incubated with 10 ng/mL mTL1A along with the antibody molecule of interest. Cells and reagents are incubated for 72 hours and supernatants are collected for cytokine analysis and cell viability is measured using Cell Titer Glo (Promega, cat#G7570).

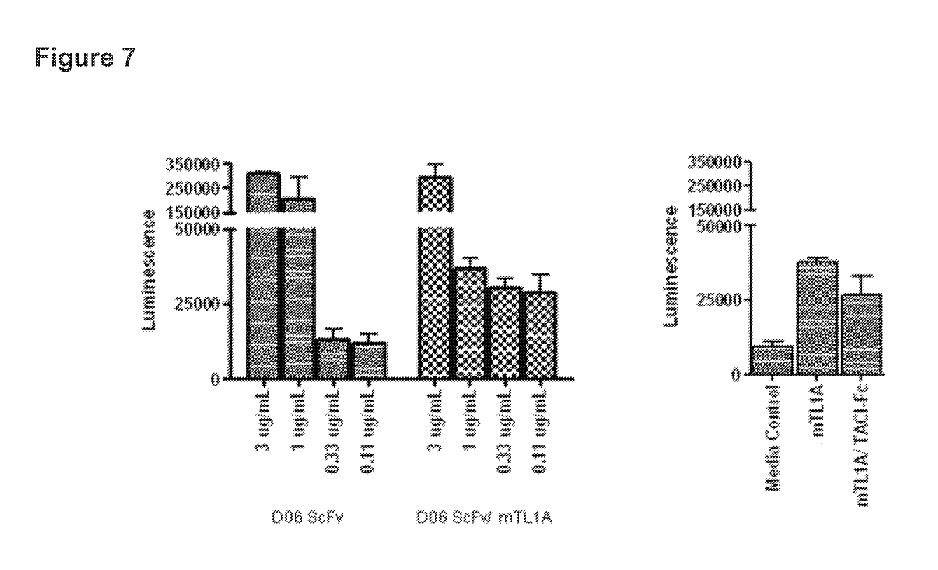

FIG. 7 shows that, similar to the 11H08 monoclonal antibody, the D06-scFv had an agonistic effect on cell proliferation from murine T cells. D06-scFv was used at concentrations of 0.11 μg/mL, 0.33 μg/mL, 1 μg/mL, or 3 μg/mL, while the control TACI-Fc was used at 1 μg/mL. Even in the absence of mTL1A, treatment with D06-scFv increased cell proliferation, as measured by luminescence.

### Example 9

### TF-1 Assay Using Anti-DR3 Fragments

In order to test the effect of 11H08-scFv-HSA and the humanized anti-DR3-Fab molecules, a TF-1 assay was used to test the ability of these various anti-DR3 fragments to block TL1A induced activity. The TF-1 assay was performed as indicated above in Example 5, but where the various anti-DR3 fragments were used instead of the whole 11H08 immunoglobulin as described.

### 11H08-Fab Blocked TL1A-Induced Apoptosis

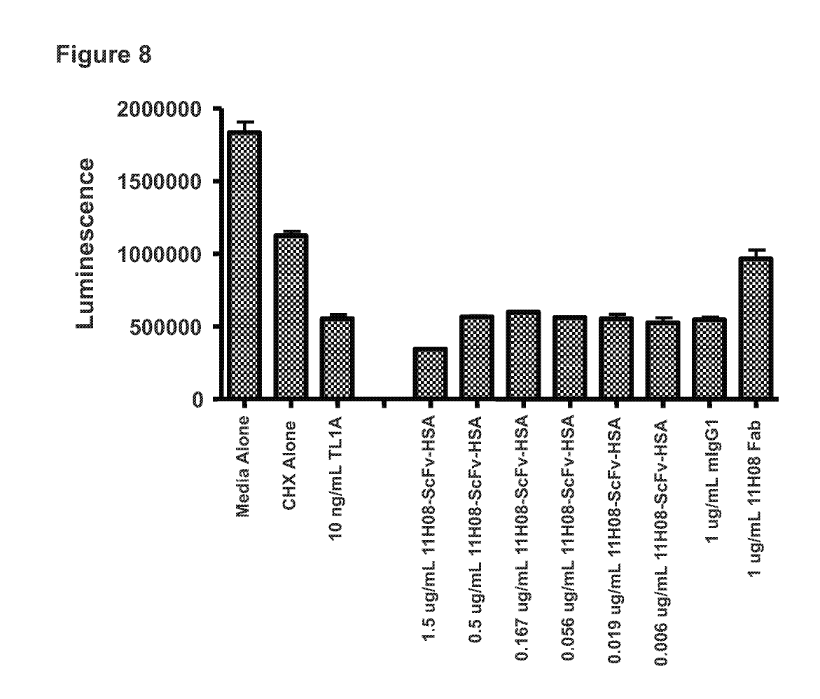

The addition of human TL1A and cycloheximide to TF-1 cells causes a decrease in TF-1 cell viability (measured by luminescence). As shown in FIG. 8, various concentrations of 11H08-scFv-HSA did not prevent TL1A-induced apoptosis. In contrast, FIG. 8 also demonstrates that a single concentration of 11H08-Fab prevented TL1A-induced apoptosis. These results indicate that the 11H08-Fab molecule inhibits TL1A binding to DR3 without activating the receptor.

### The Humanized Anti-DR3 Fab-HSA Variants Blocked TL1A Activity

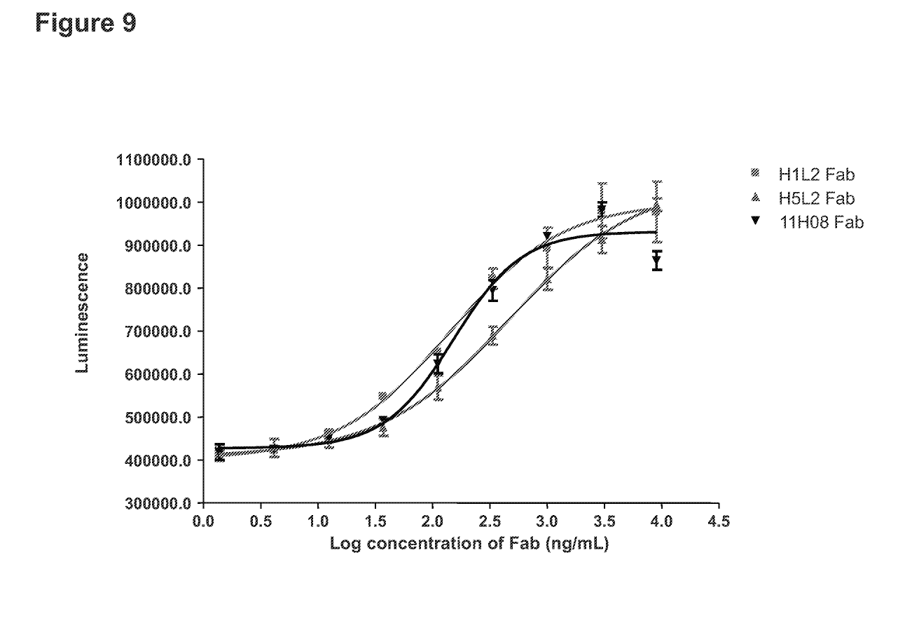

In contrast to the whole 11H08 immunoglobulin and 11H08-scFv-HSA molecules and similar to the 11H08-Fab molecule, the humanized 11H08-Fab-HSA variants, which also bind the DR3 receptor, blocked TL1A activity in this assay. As shown in FIG. 9, the anti-DR3 Fab molecules H1L2-Fab, H5L2-Fab and 11H08-Fab inhibited TL1A-induced apoptosis in TF-1 cells. These Fabs demonstrated a dose-dependent inhibition of apoptosis in cycloheximide-treated TF-1 cells in the presence of TL1A.

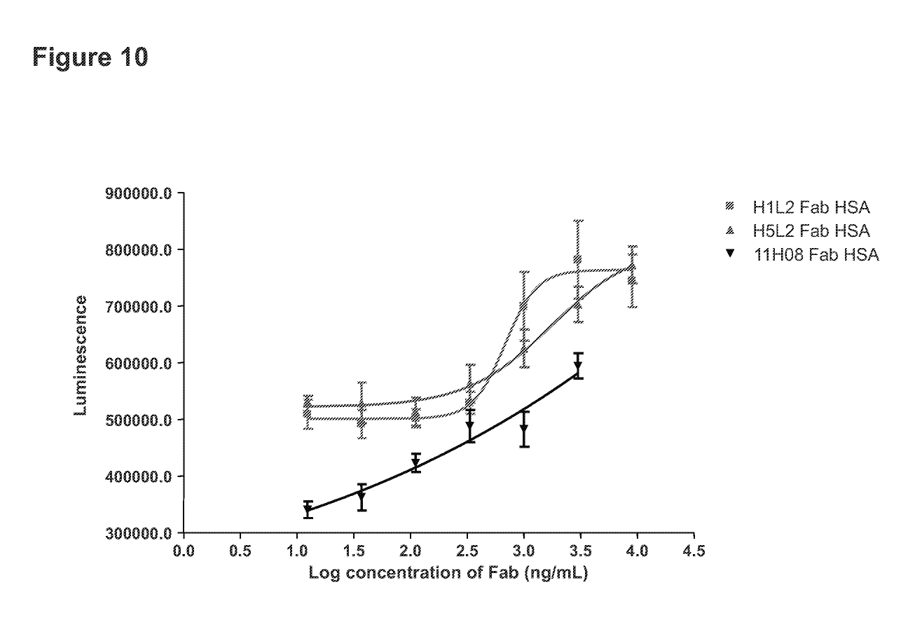

FIG. 10 demonstrates that these molecules, once fused to HSA showed the same general cell protection; moreover, the H1L2-Fab-HSA and H5L2-Fab-HSA variants inhibited comparably to the parental 11H08-Fab-HSA molecule.

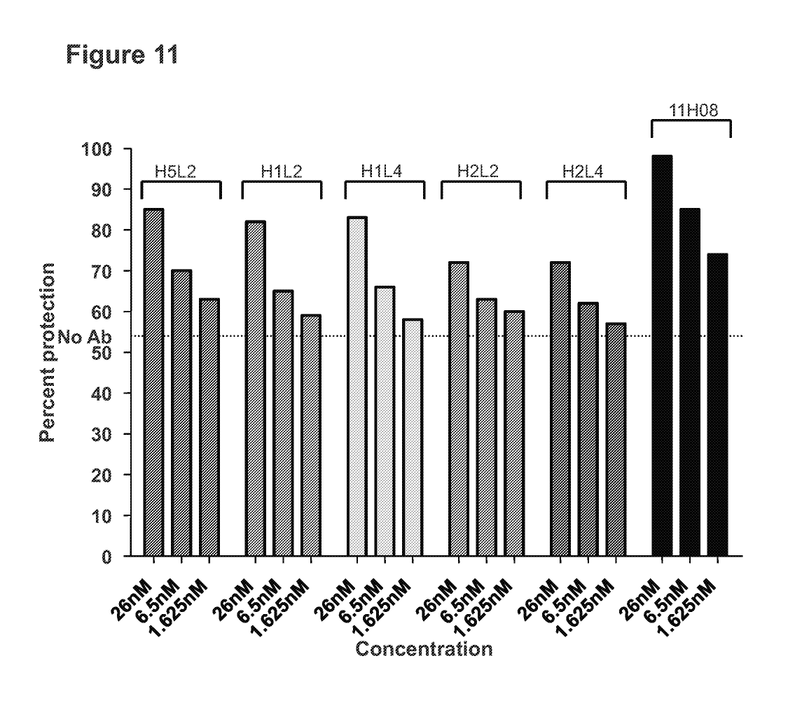

FIG. 11 is a summary graph showing that increased concentrations of the anti-DR3-Fab variants and anti-DR3-Fab-HSA variants comparably protected TF-1 cells from TL1A-induced apoptosis. All of the molecules tested showed greater cell protection from apoptosis than the baseline level of apoptosis induced by treatment with cycloheximide and TL1A alone.

### Example 10

### Evaluation of Binding Properties of Humanized Anti-DR3 Variants

Antibody binding to targets can be analyzed using a BIAcore assay as well-known in the art and as previously described (Migone et al. Immunity 16:479-492 (2002) and the BIAcore manufacturer's suggested protocol). Briefly, target proteins are covalently immoblilzed to the BIAcore CM5 sensor chip (Cat #BR-1000-14 (Biacore Life Sciences, Piscataway, N.J.)) via amine groups using N-ethyl-N′-(dimethylaminopropyl) carbodilimide/N-hydroxysuccinimide chemistry. Empty flow cells can be used as controls negative for target binding and for background subtraction. Six different concentrations of antibody molecules (range 0 to 50 nM) are flowed over the polypeptide-derivatized flow cells at 15 μL1/min for a total volume of 50 μl. The amount of bound protein is determined during washing of the flow cell with HBS buffer (10 mM HEPES [pH 7.4], 150 mM NaCl, 3.4 mM EDTA, and 0.005% Surfactant P20). The flow cell surface is regenerated by displacing bound protein by washing with 20 μl of 10 mM glycine-HCL (pH 2.3). For kinetic analysis, the on and off rates are determined using the kinetic evaluation program in BIAevaluation 3 software using a 1:1 binding model and the global analysis method.

In order to confirm that the humanized variant antibodies were capable of binding to the extracellular domain of DR3, the Kd values of eight humanized Fab variants were determined by BIAcore assay. In this experiment, a human DR3-Fc fusion protein was covalently immoblilzed to the BIAcore CM5 sensor chip. Empty flow cells that were negative for anti-DR3-Fab binding were used for background subtraction. The average Kd (n=2) of each and the binding relative to the parental anti-DR3 molecule (H0L0-Fab) are listed in Table 3.

TABLE 3

| | | | | Ratio of |
|---|---|---|---|---|
| | | | | binding |
| anti-DR3-Fab | Heavy Chain | Light Chain | | relative to |
| variant | SEQ ID NO: | SEQ ID NO: | Avg. Kd (M) | H0L0-Fab |
| H0L0-Fab | 7 | 20 | 1.37 × 10⁻⁹ | 1 |
| H0.1L0-Fab | 8 | 20 | 1.50 × 10⁻⁹ | 1.09 |
| H1.1L1-Fab | 10 | 21* | 1.46 × 10⁻⁹ | 1.06 |
| H1.1L2-Fab | 10 | 22* | 1.17 × 10⁻⁹ | 0.85 |
| H1.1L4-Fab | 10 | 24* | 1.29 × 10⁻⁹ | 0.94 |
| H4.2L2-Fab | 16 | 22* | 2.01 × 10⁻⁹ | 1.46 |
| H5.3L2-Fab | 19 | 22* | 1.67 × 10⁻⁹ | 1.22 |
| H5.3L4-Fab | 19 | 24* | 1.76 × 10⁻⁹ | 1.28 |

*Light chains contain Leucine at Kabat position 104, as described in Example 6.

Additionally, H1L2-Fab and H5L2-Fab were fused to human serum albumin (See, Example 6) and similarly tested. The Kd values of H0L0-Fab-HSA, H1L2-Fab-HSA, and H5L2-Fab-HSA were determined by BIAcore analysis as described above. The average Kd (n=2) and ratio of binding relative to H0L0-Fab are listed in Table 4.

TABLE 4

| | | | | Ratio of |
|---|---|---|---|---|
| anti-DR3 Variant | | | | binding |
| Fused to HSA | Heavy Chain | Light Chain | | relative to |
| (SEQ ID NO: 4) | SEQ ID NO: | SEQ ID NO: | Avg Kd (M) | H0L0-Fab |
| H0L0-Fab-HSA | 7 | 20 | 5.50 × 10⁻⁹ | 1 |
| H1L2-Fab-HSA | 9 | 22* | 3.06 × 10⁻⁹ | 0.6 |
| H5L2-Fab-HSA | 17 | 22* | 7.52 × 10⁻⁹ | 1.4 |

*Light chains contain Leucine at Kabat position 104, as described in Example 6.

### Example 11

### SEAP Assay to Measure the Ability of Anti-DR3-Fab-HSA Molecules to Block TL1A Signaling Through NF-κB

DR3-Fc is known to bind to TL1A and inhibit activity (Migone et al. Immunity 16:479-492 (2002)). TL1A has been shown to exert its activity in part by binding to the DR3 receptor expressed on the cell surface and triggering NF-κB signaling. A SEAP assay can be used to measure NF-κB expression. A SEAP assay was used to measure the ability of the anti-DR3-Fab-HSA molecules to block TL1A signaling through NF-κB.

SEAP Assay of TF-1 Cells Transfected with the NF-κB/SEAP Reporter Vector

A SEAP assay can be performed using standard methods known in the art. Briefly, the premyeloid cell line TF-1 is stably transfected with a NF-κB/SEAP (Nuclear Factor kappa-light-chain-enhancer of activated B cells/Secreted Alkaline Phosphatase) (Wen et al. JBC. 278:39251-39258 (2003); Shi et al. Biochem. Biophys. Res. Commun. 262:132-138 (1999)) reporter plasmid that responds to the NF-κB signal transduction pathway. The TF-1 clone is starved overnight in 0.2% FBS, 1% p/s/g, and 300 μg/mL neomycin. The cells are then resuspended to 150,000 cells/well in 2 mL of starvation media and 2 ng/mL GM-CSF. The cells are incubated for approximately 24 hours with or without TL1A (100 ng/mL or 1000 ng/mL) and the antibody molecule of interest.

SEAP activity is assayed using the Tropix Phospho-light Kit (Cat. BP-400) according to the following general procedure. The Tropix Phospho-light Kit supplies the Dilution, Assay, and Reaction Buffers used below.

A dispenser is primed with the 2.5× Dilution Buffer and 15 μl of 2.5× dilution buffer is dispensed into Optiplates containing 35 μl of a supernatant. The plates are sealed with a plastic sealer and incubated at 65° C. for 30 min. The Optiplates are separated to avoid uneven heating.

The samples are cooled to room temperature for 15 minutes. The dispenser is emptied and primed with the Assay Buffer. 50 μl of Assay Buffer is added and the samples are incubated at room temperature 5 min. The dispenser is emptied and primed with the Reaction Buffer. 50 μl Reaction Buffer is added and the samples are incubated at room temperature for 20 minutes.

The relative light unit in the luminometer is read and an increase in chemiluminescence indicates reporter activity.

A SEAP assay was used to measure NF-κB expression from a reporter plasmid transfected into TF-1 cells to determine the ability of the anti-DR3-Fab-HSA molecules to inhibit TL1A activity. In this experiment, cells received no antibody, 10 μg/mL of either the H1L2-Fab-HSA or the H5L2-Fab-HSA, or the control DR3-Fc. The levels of NF-κB were determined by measuring the SEAP activity.

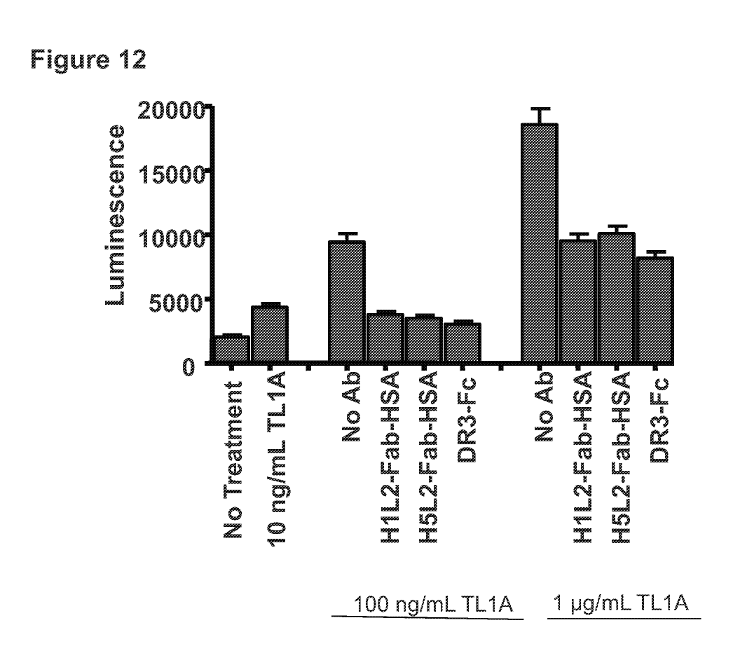

As shown in FIG. 12, both H1L2-Fab-HSA and H5L2-Fab-HSA inhibited TL1A signaling through NF-κB at both TL1A concentrations tested.

### Example 12

### Primary Human T Cell Assay to Measure Affect of 11H08-Fab-HSA on TL1A-Induced Proliferation and Cytokine Secretion

In primary human T cells, TL1A acts as a costimulator to upregulate IL2 receptor expression thereby increasing proliferation and the secretion of proinflammatory cytokines in vivo and in vitro (Migone et al. Immunity 16:479-492 (2002)).

A primary human T cell assay can be performed using standard methods known in the art. Briefly, primary human T cells are incubated with 100 ng/mL human TL1A ligand and appropriate concentrations of the antibody molecules to be tested for 72 hours. This incubation is followed by a 24 hour incubation with 5 ng/mL IL-2. Proliferation is measured by a standard luminescent protocol. The amount of cytokine secreted is determined by standard methods in the art.

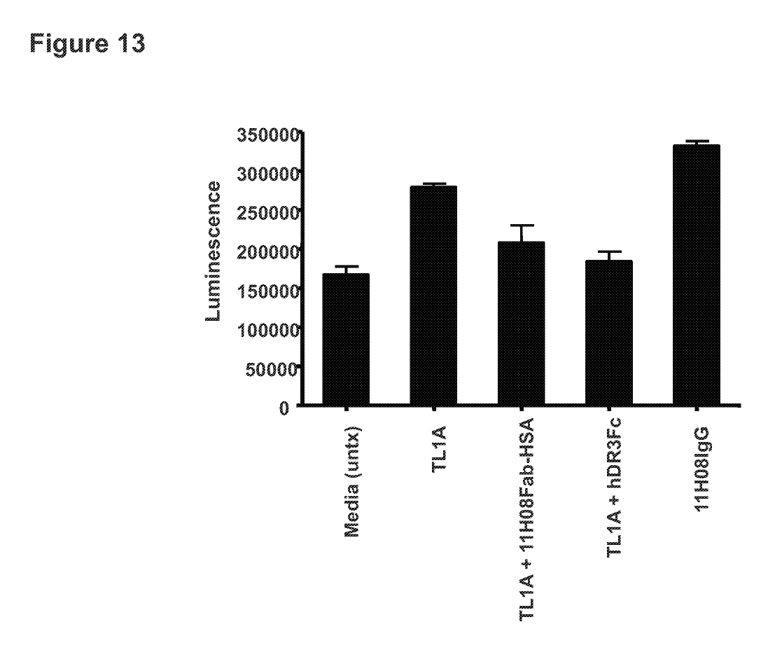

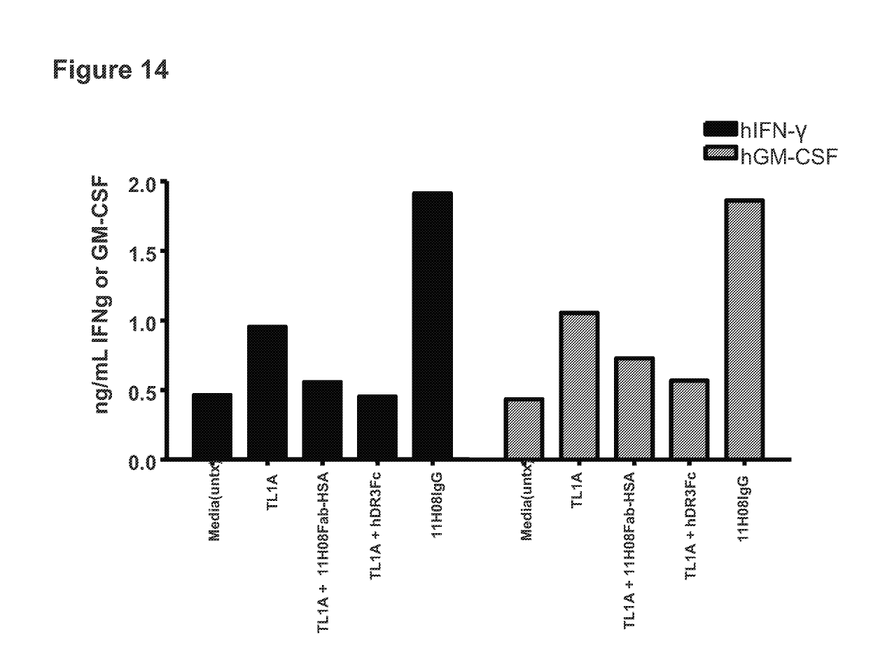

A primary human T cell assay was performed to determine the effect of the 11H08-Fab-HSA on proliferation and the secretion of IFNγ or GM-CSF from cells treated with TL1A. FIGS. 13 and 14 showed that the addition of TL1A to the cell media caused an increase in cell proliferation and an increase in cytokine secretion, which were inhibited by treatment of the cell culture with 11H08-Fab-HSA and hDR3-Fc. Conversely, the full length 11H08 immunoglobulin, which activates the receptor, induced TL1A-like activity. 3 μg/mL 11H08-Fab-HSA, 3 μg/mL hDR3-Fc, or 3 μg/mL full length 11H08 immunoglobulin were used in this experiment. Results were compared to a set of cells treated only with media and cells that were treated only with 100 ng/mL TL1A, as indicated.

### Example 13

### Anti-DR3-Fab-MSA Molecules in Murine Model of Collagen Induced Arthritis (CIA)

The development of collagen induced arthritis is known to depend on T cell activation. Additionally, Seetharaman et al. (J. of Immunology, 163:1577-1583 (1999)) have shown that inhibition of NF-κB activation in T cells impairs the development of CIA. These data indicate that impairing T cell activation by blocking DR3 activation may be an attractive avenue in treating and/or preventing arthritis. Type II collagen was used to establish an experimental model of arthritis in mice (Courtenay et al. 1980).

In general CIA was induced using the following protocol. Male DBA/1JLacJ mice (8-10 weeks) were injected intradermally (i.d.) at the base of the tail with 1000 of type II bovine collagen (CII) and complete Freund's adjuvant (CFA) at a final concentration of 4 mg/ml on Day 1. Mice were given a second injection i.d. with CII and CFA on Day 21. On days 21, 28, 35, and 42, mice were treated prophylactically with 200 μg intraperitoneal (i.p.) of the therapeutic molecules, anti-TNF-α antibody (R&D systems, cat #AB-410-NA) or D06-Fab-MSA. In addition, a control group of mice was not treated with a therapeutic molecule (“No Treatment”) and a second control group was treated with 50 μg dexamethasone i.p. every other day (e.o.d.) (“Dexamethasone”). The animals were scored every other day until the experiment was terminated on Day 46. Clinical scores were determined according to the criteria listed in Table 5.

TABLE 5

| Paw Score | Clinical Observations |
|---|---|
| 0 | Normal paw |
| 1 | One toe inflamed and swollen |
| 2 | More than one toe, but not the entire paw, inflamed and |
| | swollen, |
| | OR |
| | Mild swelling of entire paw |
| 3 | Entire paw inflamed and swollen |
| 4 | Very inflamed and swollen paw or ankylosed paw. If the |
| | paw is ankylosed, the mouse cannot grip the wire top of |
| | the cage. |

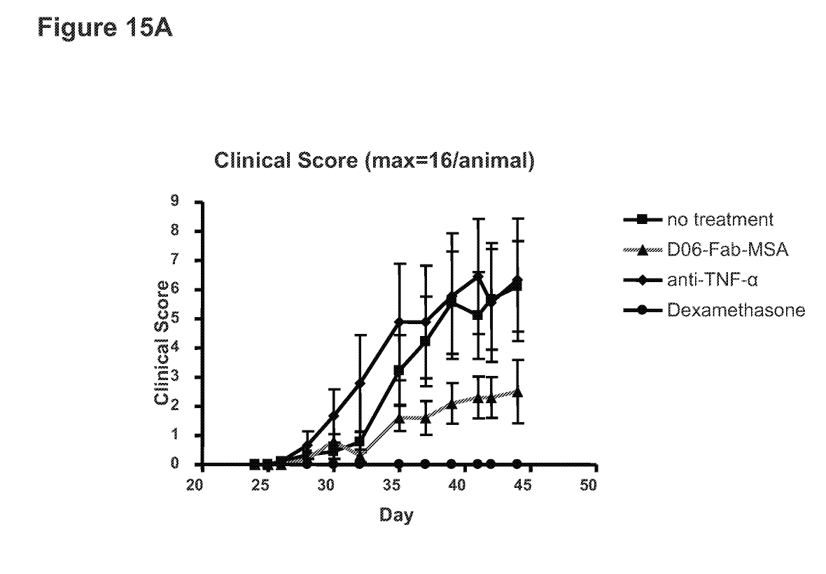

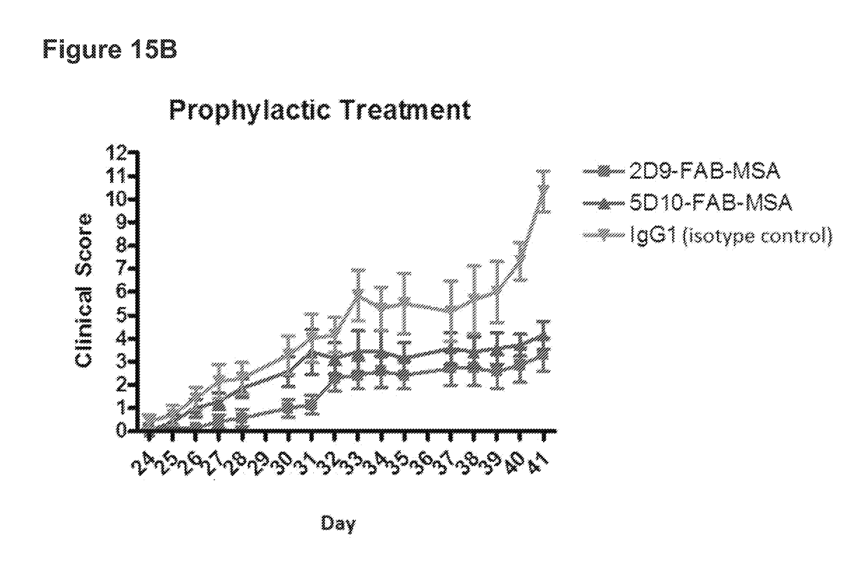

The maximum score for any one animal is 16. As shown in FIG. 15A, CIA mice treated with D06-Fab-MSA had reduced clinical observation scores as compared to untreated CIA mice and CIA mice treated with anti-TNF-α. Dexamethasone compeletely prevented clinical manifestations of collagen-induced arthritis. Table 6 further shows that treatment with D06-Fab-MSA reduced the incidence of arthritis compared to the untreated control group and the group treated with the anti-TNF-α antibody.

TABLE 6

| | | % Arthritis Incidence |
|---|---|---|
| Treatment (CIA mice) | Dosage administered i.p. | at Day 42 |
| No Treatment | — | 90 |
| D06-Fab-MSA | 200 μg | 50 |
| Dexamethasone | 50 μg | 0 |
| anti-TNF-α | 200 μg | 80 |

In a separate experiment, on days 21, 28, 35, and 42, CIA-mice were treated prophylactically with 200 μg intraperitoneal (i.p.) of either 2D9-Fab-MSA, or 5D10-Fab-MSA. In addition, a control group of mice was treated with an isotype control antibody (IgG1). The animals were scored every other day until the experiment was terminated on Day 46. Clinical scores were determined as above. As shown in FIG. 15B, CIA mice treated with 2D9-Fab-MSA and 5D10-Fab-MSA had reduced clinical observation scores as compared to isotype-control treated CIA mice.

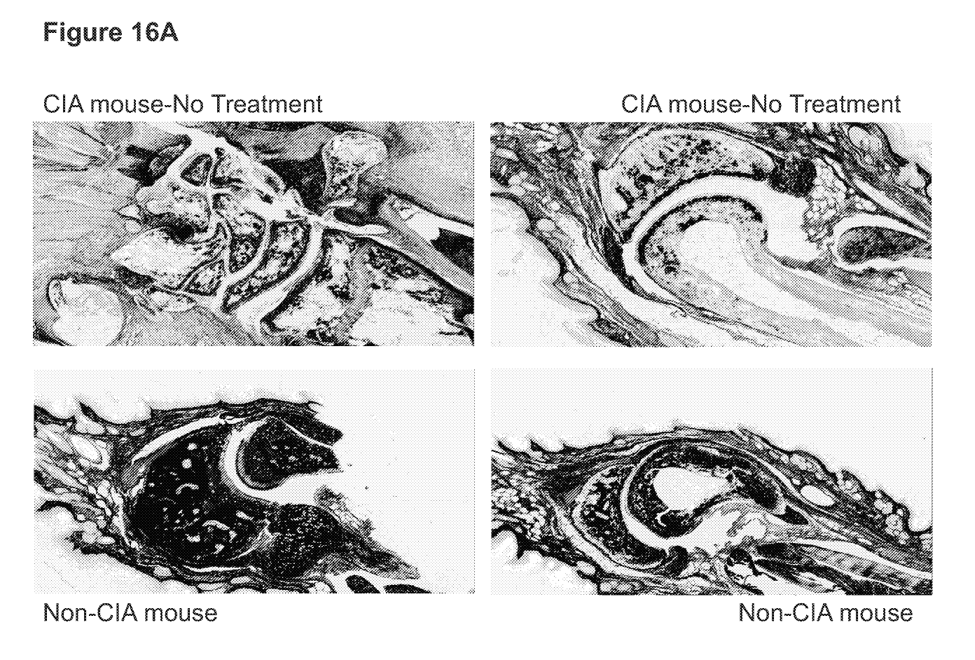

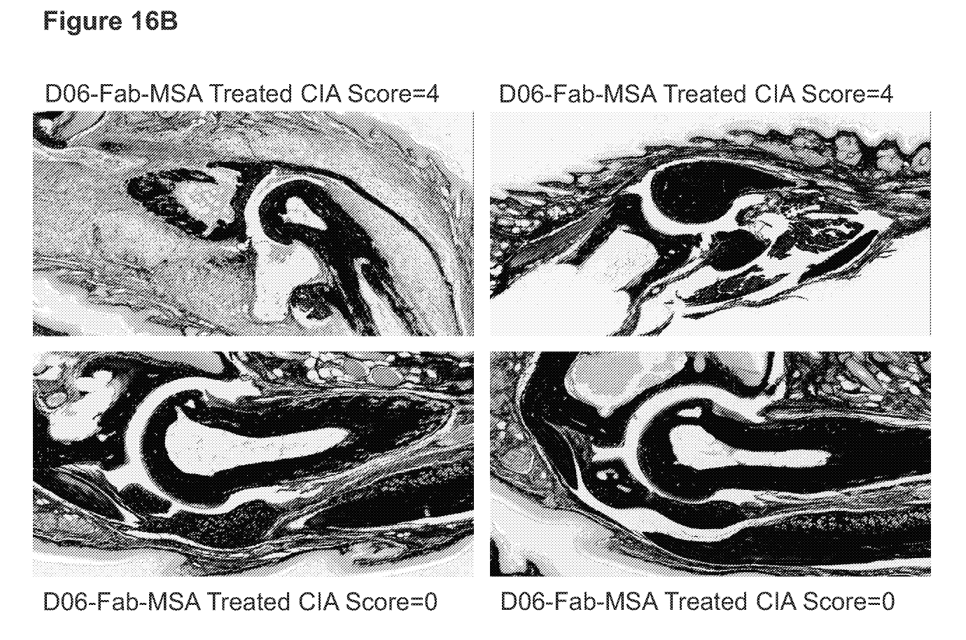

Histology was preformed to further determine the degree of inflammation as well as potential bone erosion in these animals. Paws were removed from the animals at the study's termination and placed in 10% neutral buffered formalin. These paws were subsequently stained with trichrome to look for bone erosion and inflammation (FIGS. 16A and 16B). Trichrome will stain blue in bone from non-CIA mice, which indicates that there is no bone erosion. By contrast, red staining is an indication of bone erosion. For purposes of presentation, the Trichrome images were converted to grayscale monochrome images, where blue staining appears as black in the images while red staining appears as light gray in the images. Little to no bone erosion was observed in the non-CIA mice (score=0) as well as the CIA mice treated with D06-Fab-MSA (score=0). Some of the CIA mice treated with D06-Fab-MSA had one or more paws that scored a 3 or 4, but did not have the significant bone erosion as seen in the untreated animals as indicated by the significant light gray (red) staining in the joints. The light gray (red) staining is absent on the outside of the bones in the non-CIA mice and D06-Fab-MSA animals (score=0).

Additionally, no inflammation was observed in those mice treated with D06-Fab-MSA that showed no clinical CIA symptoms, while inflammation was observed in both treated and untreated animals that showed clinical symptoms. Thus, results indicate that the D06-Fab-MSA is capable of reducing inflammatory symptoms.

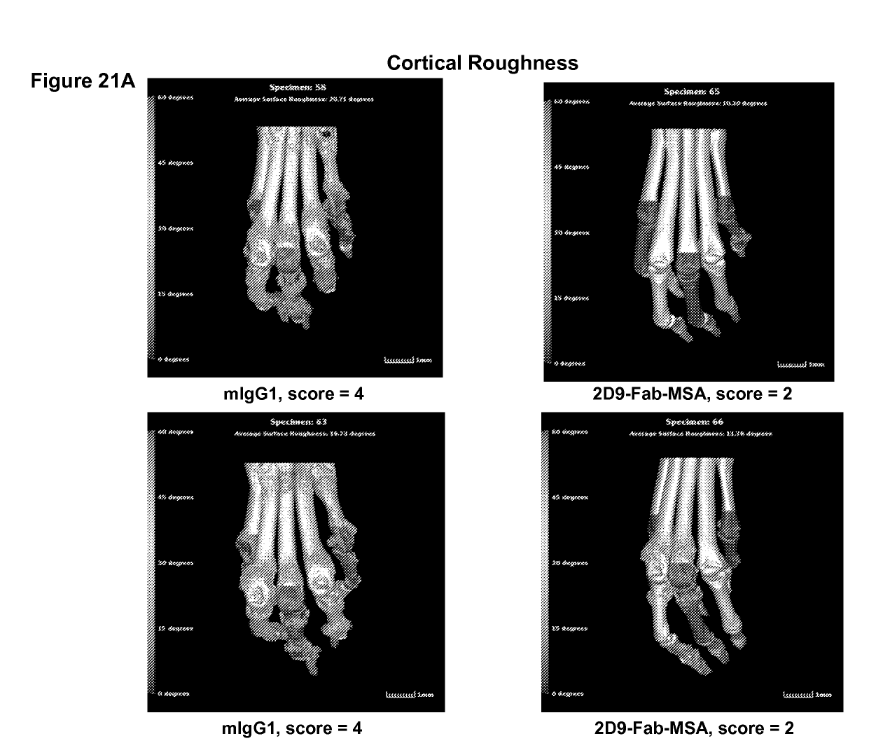

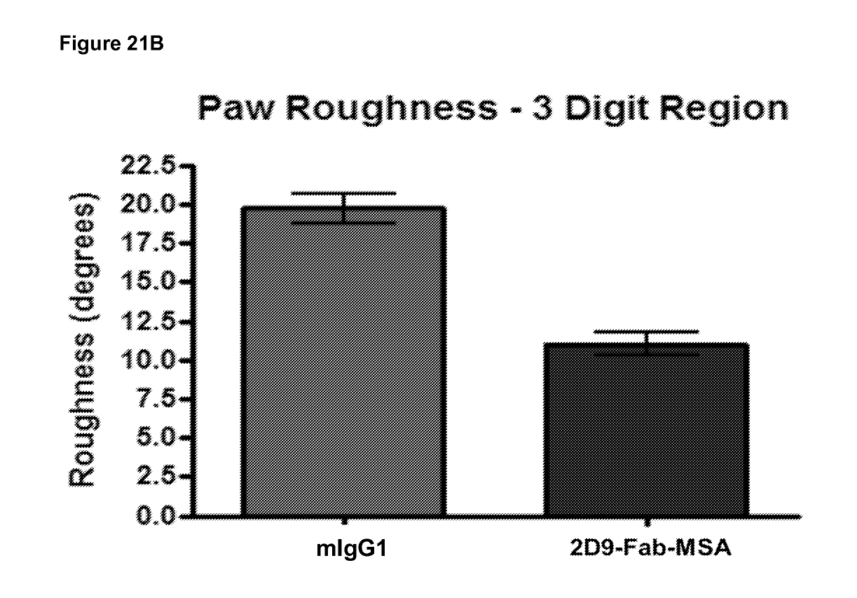

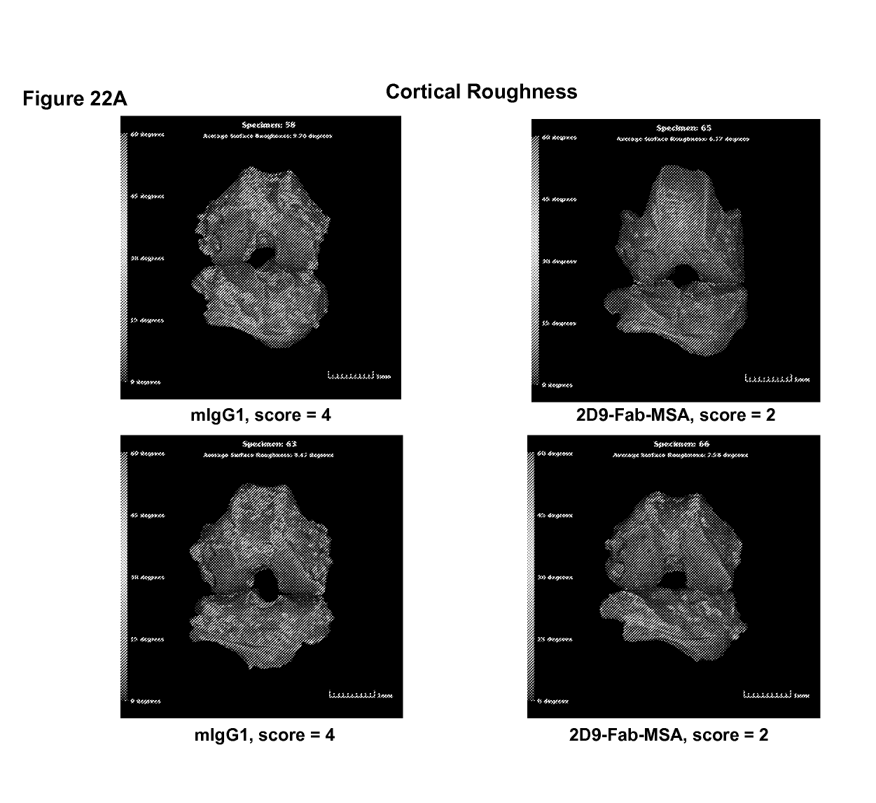

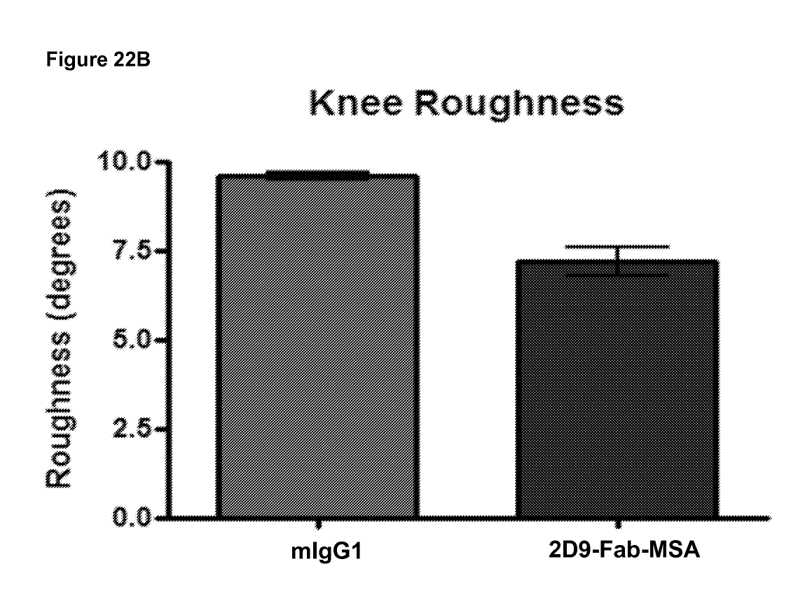

In a separate experiment, the paws and joints of treated and untreated mice were analyzed to determine whether 2D9-Fab-MSA treatment decreased cortical roughness in CIA-mice. Mouse femurs were stored at −80° C. until processing. Samples were scanned at Numira (Salt Lake City, Utah, USA) using a high-resolution, volumetric pCT40 scanner (Scanco Medical AG, Basserdorf, Switzerland). The image data were acquired at 6 μm isometric voxel resolution with 300 ms exposure time, 2,000 views, and five frames per view (FIGS. 21A and 22A). The micro-computer tomography-generated DICOM files were used to analyse the samples and to create volume renderings of the region of interest. The raw data files were viewed using Microview (GE Healthcare, Milwaukee, Wis., USA).

Utilizing ScanCo Medical software, separate bone density measurements were obtained for the paws and knee joint. Three-dimensional image rendering were generated through original volumetric reconstructed images using Microview software (GE Healthcare, Piscataway, N.J., USA). Cortical roughness measurements were obtained from the paw by taking the average of three tarsi and then the average of two separate specimens. For example, three tarsi from specimen #58 (isotype control) were measured for cortical roughness. This procedure was repeated for specimen #63 and then average of the cortical roughness scored was acquired from #58 and #63 (FIG. 21B). The same procedure was utilized to determine cortical roughness for the knees (FIGS. 22A and B). These data are also shown below in Tables 7 and 8.

TABLE 7
Paw 3 Digit Region

| | Volume | Surface | Roughness |
|---|---|---|---|
| Specimen | (mm³) | Area (mm³) | (degrees) |
| HPS0151 #58-mIgG1 | 1.987 | 42.353 | 20.710 |
| HPS0151 #63-mIgG1 | 2.489 | 49.741 | 18.730 |
| HPS0151 #65-2D9-Fab-MSA | 2.855 | 32.813 | 10.300 |
| HPS0151 #66-2D9-Fab-MSA | 2.514 | 36.240 | 11.780 |

TABLE 8
Knee Region

| | Volume | Surface | Roughness |
|---|---|---|---|
| Specimen | (mm³) | Area (mm³) | (degrees) |
| HPS0151 #58-mIgG1 | 3.452 | 100.520 | 9.704 |
| HPS0151 #63-mIgG1 | 4.338 | 116.348 | 9.470 |
| HPS0151 #65-2D9-Fab-MSA | 6.501 | 115.774 | 6.765 |
| HPS0151 #66-2D9-Fab-MSA | 5.494 | 103.850 | 7.582 |

These data demonstrate that treatment of CIA-mice with 2D9-Fab-MSA, in addition to reducing the symptoms of arthritis, significantly reduces cortical roughness of the paw and knees in the treated animals compared with the isotype control.

### Example 14

### Measurement of Anti-Collagen Antibodies in the Serum of CIA-Mice

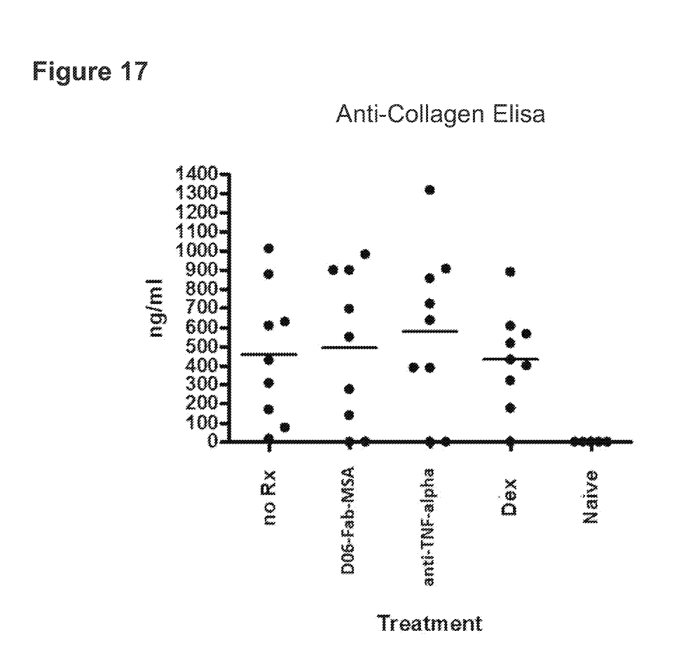

The levels of anti-collagen antibodies in the serum of CIA-mice and naïve mice were measured by ELISA. Whole blood was collected by cardiac exsanguination at termination and serum was isolated. Serum was analyzed for anti-collagen type II levels using the mouse IgG anti-collagen type II ELISA kit (MD Bioproducts), according to the manufacturer's instructions. Briefly, a microwell plate was prepared with wells coated with either mouse collagen type II or standards. 100 μl of either the Standard or diluted samples were pipetted in duplicate into the wells then the plate was covered with a plate sealer and incubated for 1 hour at room temperature. The wells were aspirated and washed 3 times with 200 μl per well of 1× Wash Buffer and 100 μl of 1× Detection Antibody were added to each well. The plate was covered with a plate sealer and incubated in the dark for 1 hour at room temperature. Again, the wells were aspirated and washed 3 times with 200 μl per well of 1× Wash Buffer. After 100 μl of Substrate was added to each well, the plate was incubated in the dark for 15 minutes at room temperature. 100 μl of Stop Solution was then added to each well and the plate was read at 450 nm. FIG. 17 shows that, when compared with naïve mice, increased systemic amounts of anti-collagen antibodies were found in the serum of CIA mice regardless of treatment.

### Example 15

### D06-Fab-MSA Effects in a Murine MOG-EAE Model

Experimental Allergic Encephalomyelitis (EAE) is widely used as a central nervous system autoimmune disease model, which mimics pathology of human multiple sclerosis. There are several variations of EAE models depending on mouse strains and immunizing reagents, for example myelin oligodendrocyte glycoprotein (MOG)-induced EAE and proteolipid protein (PLP)-induced EAE. The MOG-EAE model was used to evaluate the effects of D06-Fab-MSA in this system.

The disease model was induced by subcutaneous injection of a MOG35-55 emulsion. Each mouse received two injections near right and left inguinal lymph nodes.

MOG35-55 emulsion is prepared using the following procedure. MOG peptide is dissolved in phosphate buffered solution. 800 μg of heat-killed mycobacterium tuberculosis is added to 100 μL of complete freund adjuvant (CFA). Equal volume of MOG peptide solution and CFA are emulsified at 4° C. using a homogenizer. Mixed solution is emulsified until it becomes white and viscous. A final amount of 100 μg MOG35-55 is injected into each mouse to induce MOG-EAE. 200 μL of pertussis toxin (1.67 μg/mL) is administered via intraperitoneal injection on day 0 and day 2. Animals are weighed on day 0 and every three or four days thereafter. In general, clinical signs are monitored every other day from day 0, and daily after day 9. Clinical symptoms are scored based on the rating scale shown in Table 9.

TABLE 9

| Grade | Clinical symptoms |
|---|---|
| 0 | No abnormality |
| 1 | Flaccid tail |
| 2 | Paraparesis: weak hind limbs and inability to right itself |
| 3 | Hind limb paralysis: Inability to move hind limbs |
| 4 | Quadraplegia: Inability to move front and hind limbs |
| 5 | Moribund |

The MOG-EAE model was used to test whether D06-Fab-MSA treatment can ameliorate the symptoms of EAE, where 60 mice were separated into 6 groups as shown in Table 10.

TABLE 10

| | | Dosage | |
|---|---|---|---|
| Group | | Administered | |
| (n = 10) | Therapeutic | via IV | Administered on Day(s) |
| Naïve | — | — | — |
| MOG-EAE | control | 10 mg/kg | d-1, d 6, d 13 |
| | antibody | | |
| MOG-EAE | D06-Fab-MSA | 10 mg/kg | d 13, d 17 |
| | | | (therapeutic |
| | | | administration) |
| MOG-EAE | D06-Fab-MSA | 10 mg/kg | d-1, d 6, d 13 |
| | | | (prophylactic |
| | | | administration) |
| MOG-EAE | mDR3-Fc | 10 mg/kg | d-1, d 6, d 13, d 17 |
| | | | (prophylactic |
| | | | administration) |
| MOG-EAE | — | — | — |

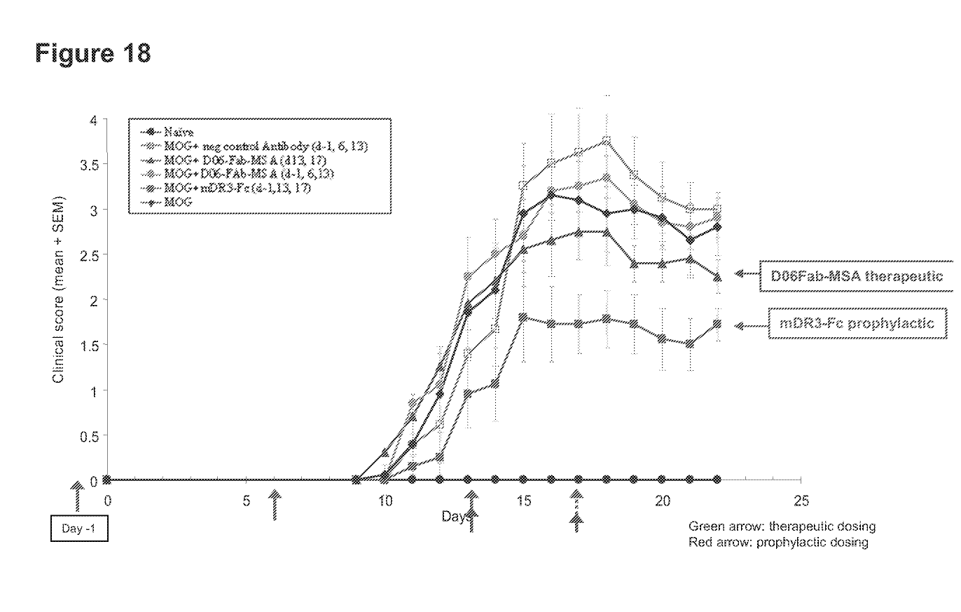

As seen in FIG. 18 and Table 11, blocking DR3 with either prophylactic or therapeutic treatment with D06-Fab-MSA suppressed the onset and severity of clinical symptoms in the murine MOG-EAE model.

TABLE 11

| | Disease | | | |
|---|---|---|---|---|
| | Incidence | | | Max Clinical |
| | #disease/ | Total | Onset (day) | Score |
| Group | total # | Burden | mean +/− SEM | mean +/− SEM |
| Naïve | 0 | 0 | 0 | 0 |
| MOG + | 4/4* | 31.4 ± 1.6 | 12.0 ± 0.6 | 3.9 ± 0.1 |
| control Ab | | | | |
| MOG + | 9/10 | 26.6 ± 3.3 | 11.4 ± 0.5 | 3.2 ± 0.2 |
| D06-Fab- | | | | |
| MSA-T | | | | |
| MOG + | 10/10 | 30.75 ± 1.8 | 11.5 ± 0.5 | 3.6 ± 0.1 |
| D06-Fab- | | | | |
| MSA-P | | | | |
| MOG + | 6/9* | 15.6 ± 4.4 | 14.2 ± 1.6 | 2.9 ± 0.3 |
| mDR3-Fc-P | | | | |
| MOG | 10/10 | 28.75 ± 1.8 | 12.1 ± 0.3 | 3.45 ± 0.1 |

*6 found dead after 3 doses possibly due to immungenicity in the control antibody group. 1 found dead in mDR3-Fc.

As can be seen in the data in Table 11, the severity of EAE symptoms were affected by whether the D06-Fab-MSA was administered before or after the mice began to manifest EAE symptoms.

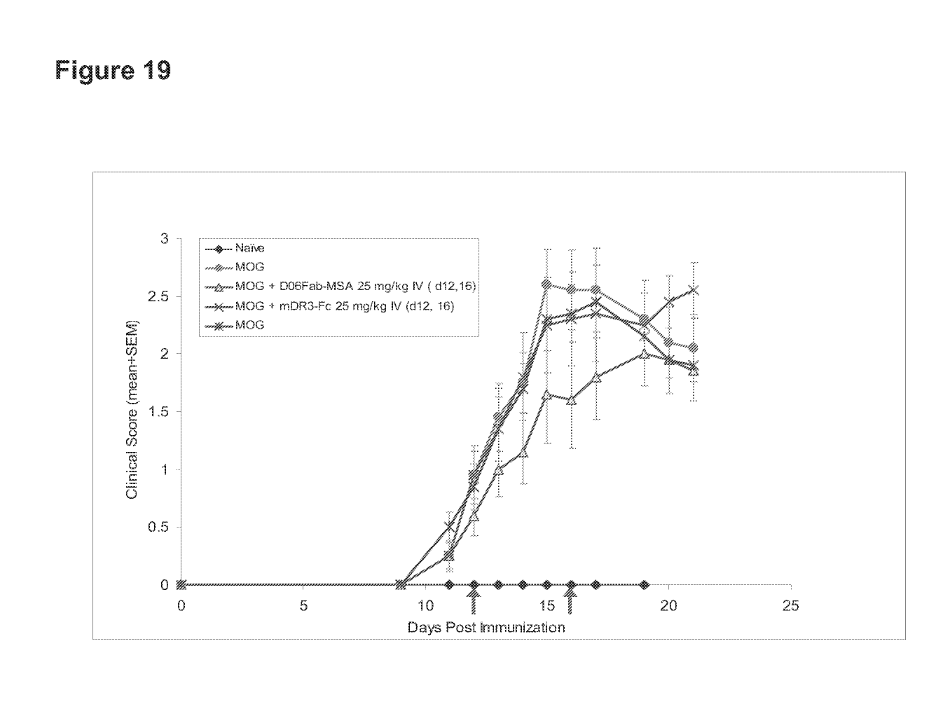

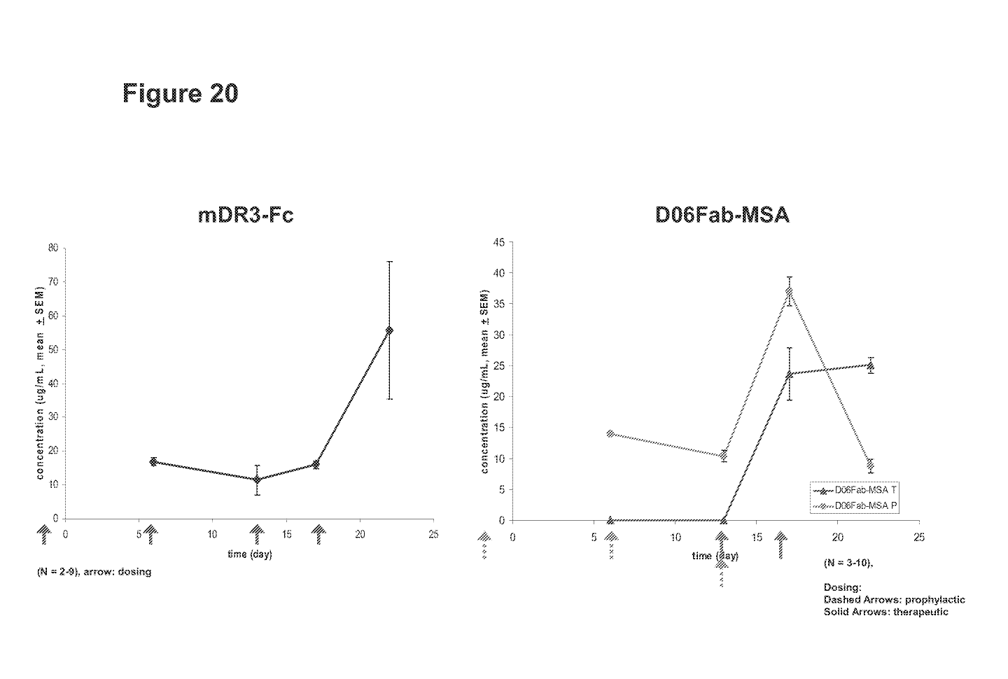

FIG. 19 shows that when the agents were administered therapeutically, the D06-Fab-MSA ameliorated EAE symptoms. Furthermore, FIG. 20 demonstrates that the D06-Fab-MSA molecule was detected and sustained its systemic level over 6 days.

### Example 16

### 2D9-Fab-MSA Treatment Effects in a Murine DSS-colitis Model

The Dextran Sulfate Sodium (DDS)-induced colitis model is widely used to evaluate potential treatment for human inflammatory bowel diseases. Exposure to DSS causes damage to the gut epithelium, thereby allowing enteric bacteria to enter and trigger a cascade of inflammatory reaction in the gut. DSS-colitis can be induced in mice by replacing the regular water in the mouse cage with a preparation of 3% (w/v) DSS. Depending on the duration of DSS exposure and the number of cycles of DSS administration, the DSS-colitis model can be acute or chronic. For the acute DSS-colitis model, mice were given water containing 3% DSS from days 0 to 7. Fresh 3% DSS water was replaced on day 4, and the regular water was returned to the cage from day 7. During the experimental period, animal body weight is measured as a parameter for colitis development. In this model, exposure to DSS led to significant weight loss over 10 days.

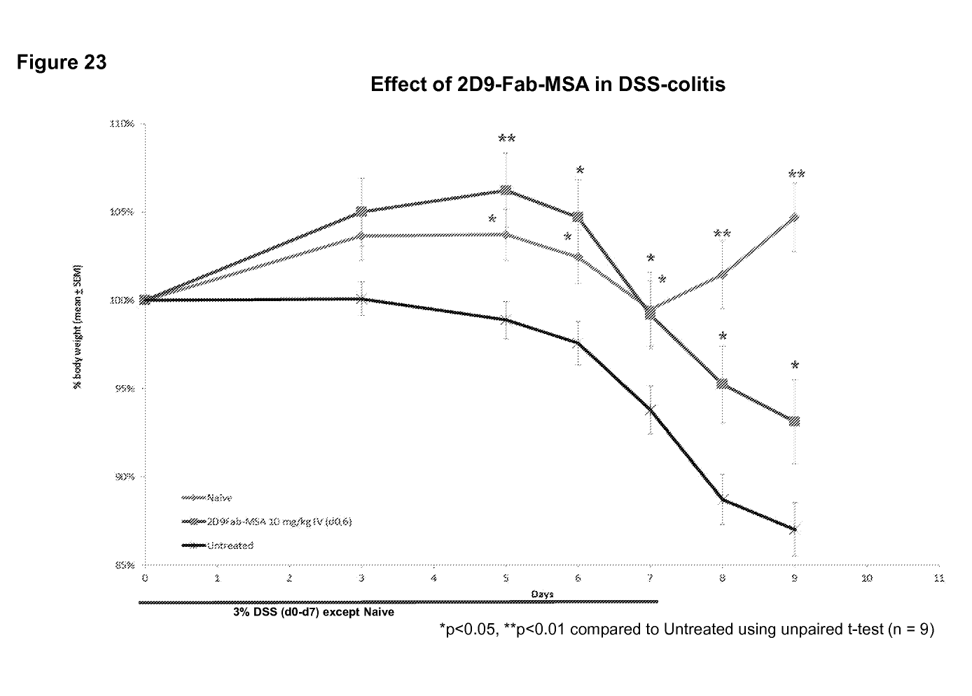

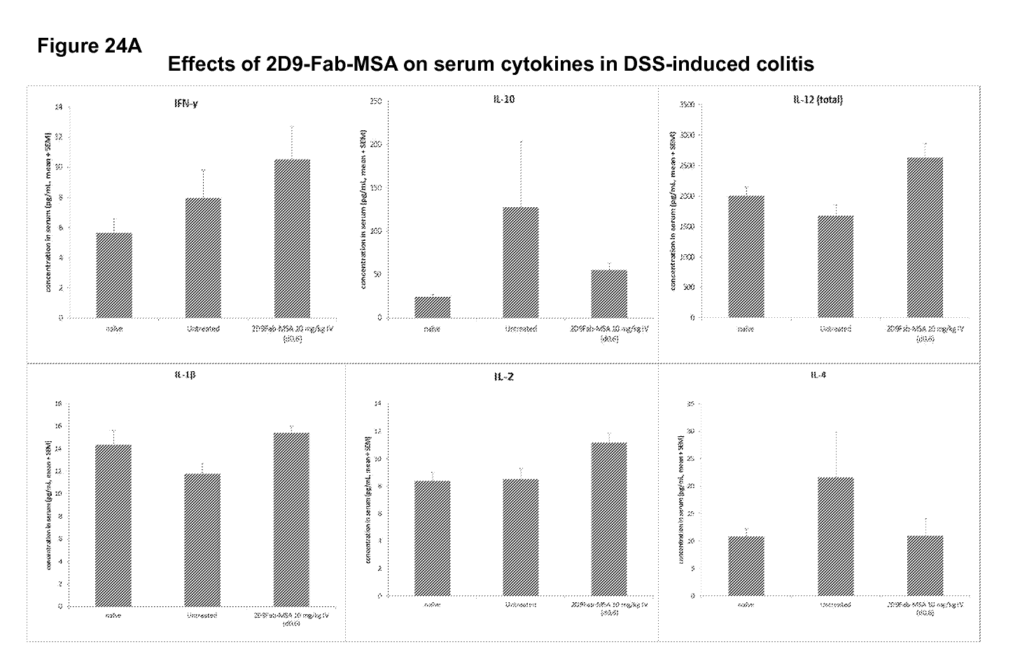

Eighteen mice were separated into 3 groups; naïve, treated, and untreated. The naïve group was watered with regular water and received no DSS water. The treated group was given 3% DSS water as described above and were also treated with 2D9-Fab-MSA (10 mg/kg) administered intravenously on days 0 and 6. The last group of untreated mice was given the 3% DSS water but received no treatment. As shown in FIG. 23, 2D9-Fab-MSA treatment significantly protected animals from DSS-induced weight loss compared to the untreated group. The 2D9-Fab-MSA treated mice maintained their body weight through day 7 and lost only 7% of their initial body weight by day 9, whereas the untreated group lost 13% of their initial body weight by day 9.

### Method for Serum and Cecum Homogenate Cytokine Assay

In addition to measuring the body weight of treated and untreated DSS-colitis mice, a cytokine assay was conducted to determine changes in cytokine levels associated with 2D9-Fab-MSA treatment. Serum and cecum samples were collected and stored at −80° C. For the cecum homogenate, the tissue was homogenized with a tissue homogenizer in the 500 uL buffer containing 0.5M Tris, pH 8.0 0.1% Triton X, and 1% BSA. Homogenates were centrifuged to collect soluble fractions from insoluble pellets and were stored at −80° C. Nine Th1/Th2 cytokines (IFN-γ, IL-1b. IL-2, IL-4, IL-5, IL-10, IL-12, TNF-α, and CXCL-1) were measured using an electrochemiluminescence detection multiplex cytokine kit (Meso Scale Discovery) which enables simultaneous measurement of multiple cytokines in a single well on a 96-well plate. Undiluted serum samples were analyzed according to the manufacturer's instructions.

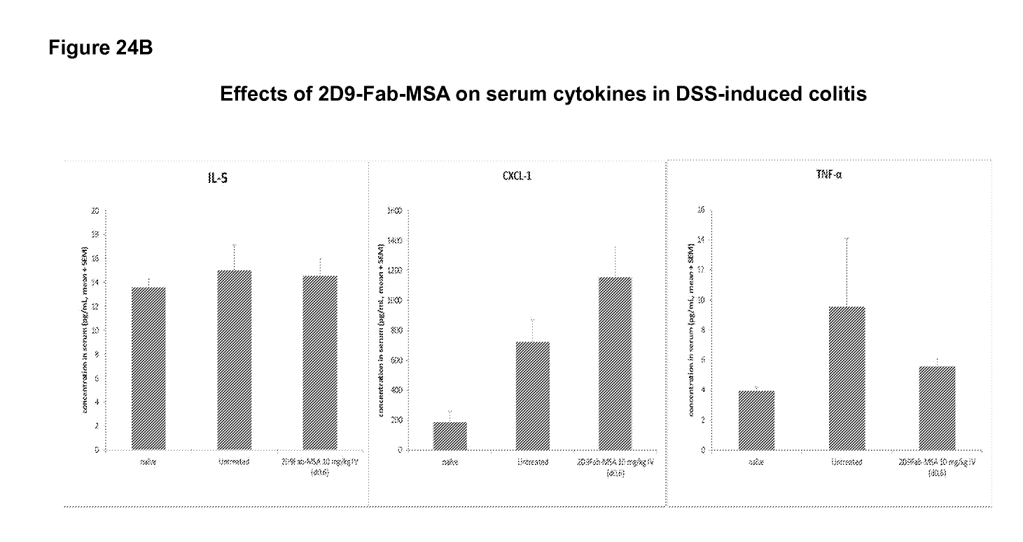

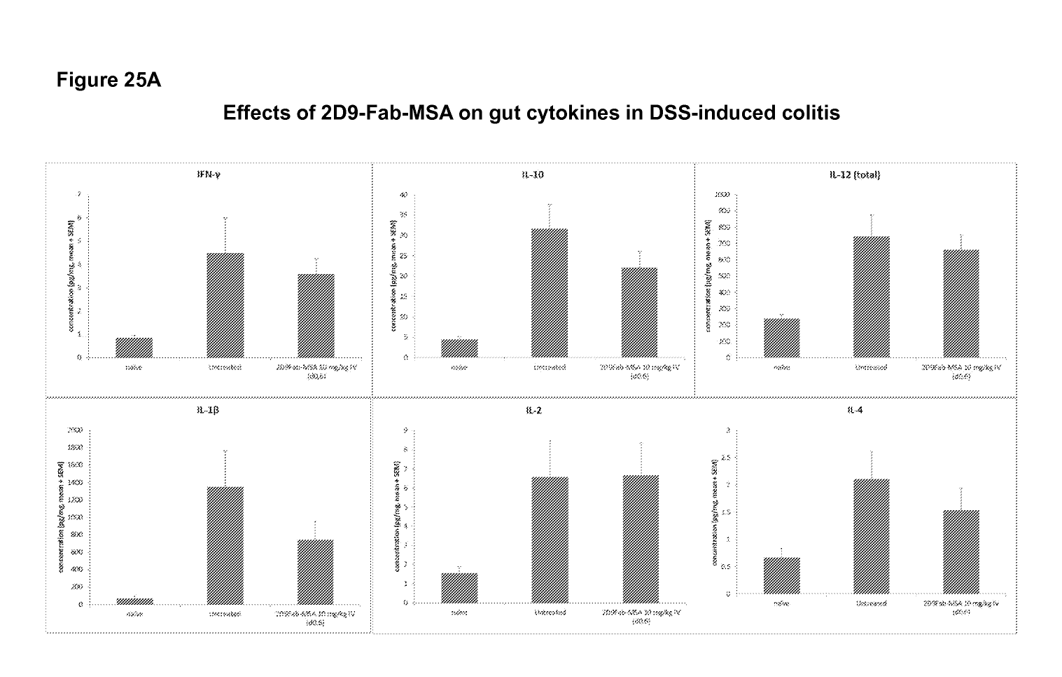

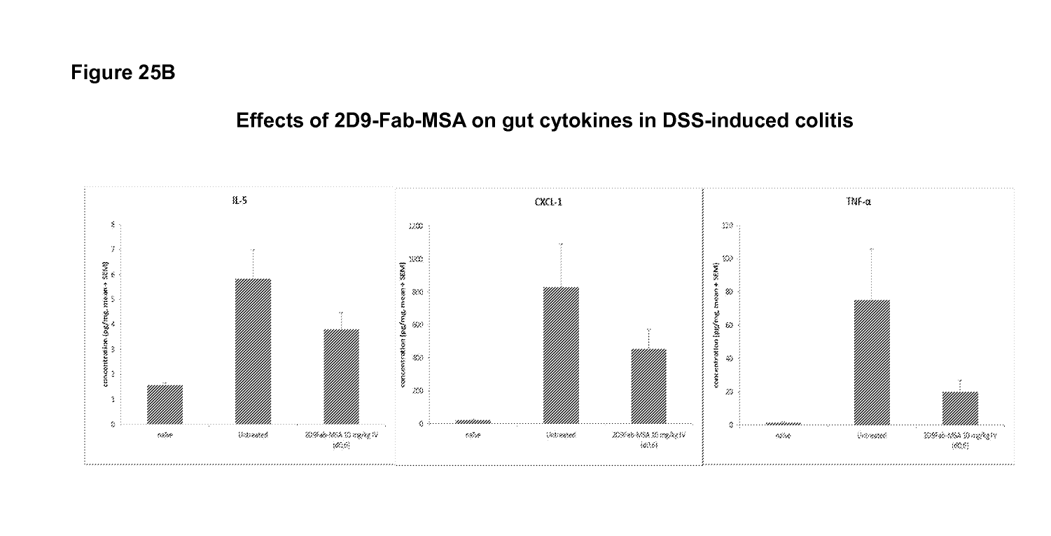

As shown in FIGS. 24A and B, serum levels of IL-10, IL-4, CXCL-1, and TNF-α were increased in the serum of animals with DSS-induced colitis. Treatment with 2D9-Fab-MSA resulted in IL-10 and TNF-α serum levels about 30% lower than in the untreated animals. Outliers beyond the range of 2×SD were excluded in the analysis based on Chauvenet's criterion (n=7-9). Additionally, FIGS. 25A and B show the effects of 2D9-Fab-MSA treatment on the levels of these cytokines in the gut. All nine cytokines were elevated 3 to 50 fold in mice with colitis compared to naïve group. Treatment of the DSS-colitis mice with 2D9-Fab-MSA reduced most of the Th1/Th2 cytokines tested, except IL-2. Reduction of cytokine levels ranged from 17% to 75% in response to 2D9-Fab-MSA treatment. Outliers beyond the range of 2×SD were excluded in the analysis based on Chauvenet's criterion (n=8-9).

### Example 17

### 2D9-Fab-MSA Treatment Effects in a Murine Adoptive Transfer Colitis Model

### Method for Inducing Adoptive Transfer Colitis in Mice

The adoptive transfer colitis model is a common chronic colitis models where colitis is induced in mice by transferring CD4+CD45RBhi cells, isolated from donor spleen and lymph nodes, to recipient severe combined immunodeficient (SCID) mice. Briefly, spleens and lymph nodes from donor Balb/c mice were dissociated using a 100 um mesh filter and a syringe plunger. The cells were then washed in PBS and enriched in CD4+ T cells using the CD4+ T cell enrichment kit (Stemcell Inc.). Enriched CD4+ T cells were stained for CD25 and CD45Rb expression, and further sorted to collect CD45Rb^{hi} cells. To remove any CD4+CD25+ cells (regulatory T cells), CD25 negative selection was added during the sorting. To generate consistent colitis in recipient mice, stringent gating was applied to isolate CD45Rb high expressing T cells. Each mouse received sorted 0.5×10e6 CD4+CD25-CD45Rb^{hi} cells intravenously on days 0 and 7. Once administered, the transferred T cells infiltrate into the gut, leading to colitis with weight loss typically starting 4 weeks post injection.

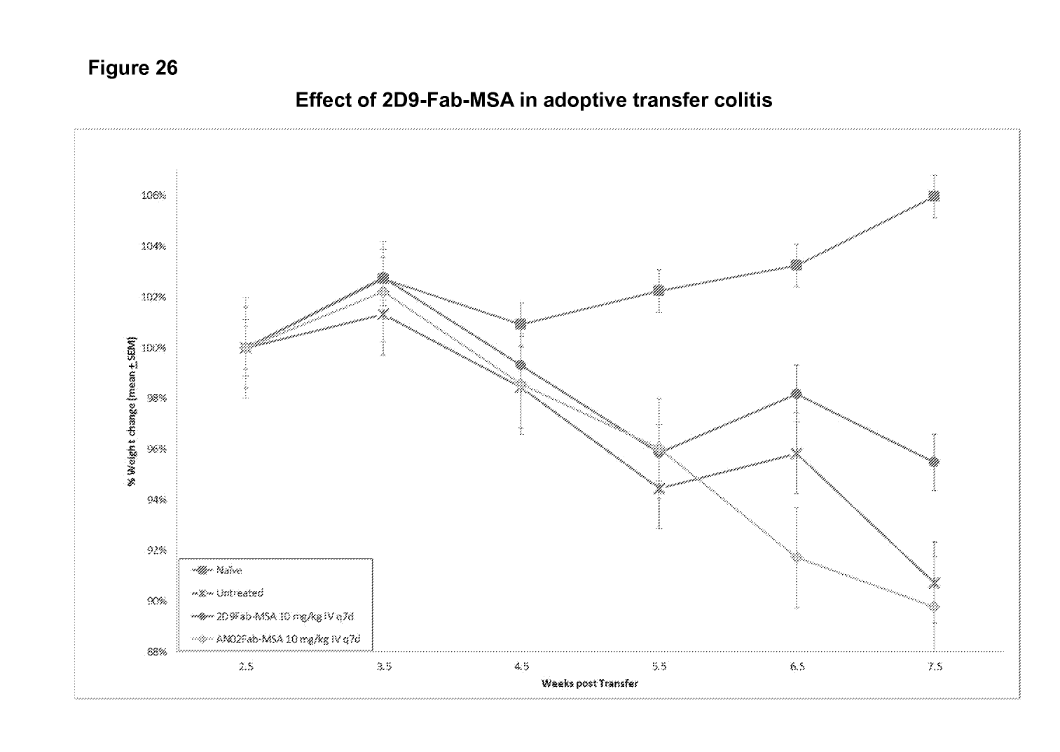

In one experiment, half a million CD4+CD25-CD45Rb^{hi} cells isolated from spleen and lymph nodes of donor Balb/c mice were intravenously injected into recipient SCID mice on days 0 and 7. The recipient SCID mice started to lose body weight 4 weeks after the first transfer. As shown in FIG. 26, weekly IV administration of 2D9-Fab-MSA (10 mg/kg) starting from 2.5 weeks, protected the treated mice from weight loss over 7.5 weeks. Conversely, administration of the isotype control, AN02-Fab-MSA, (10 mg/kg) did not prevent colitis progression and weight loss.

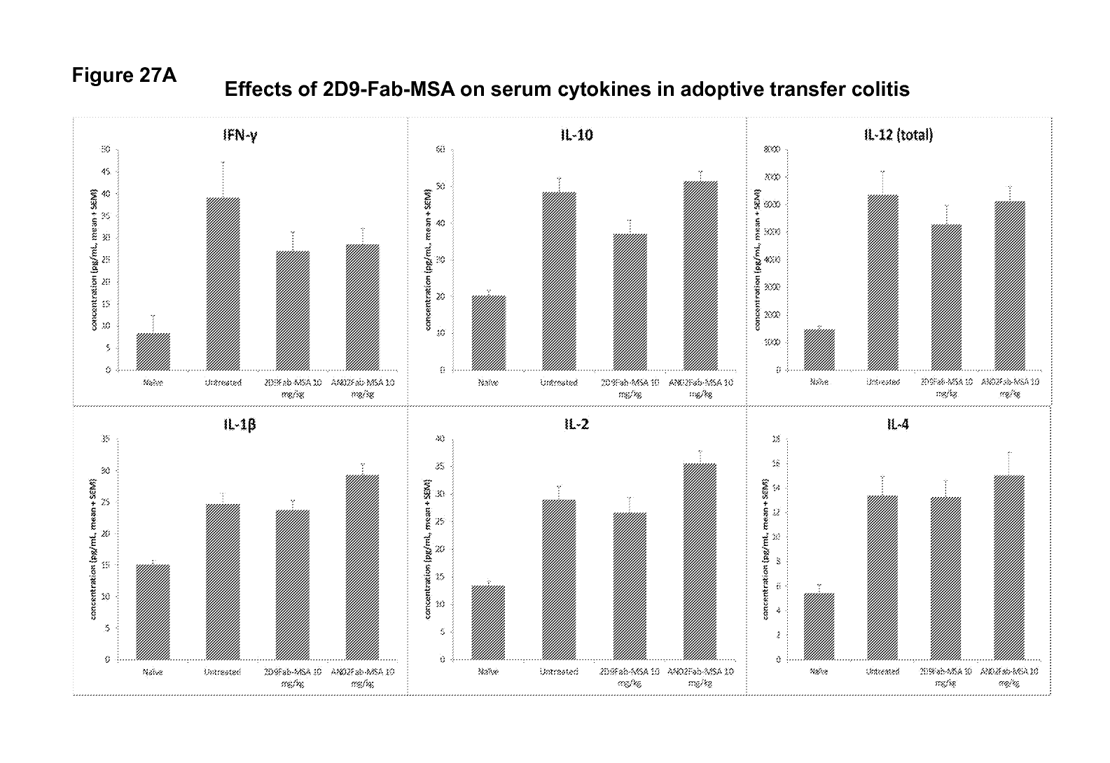

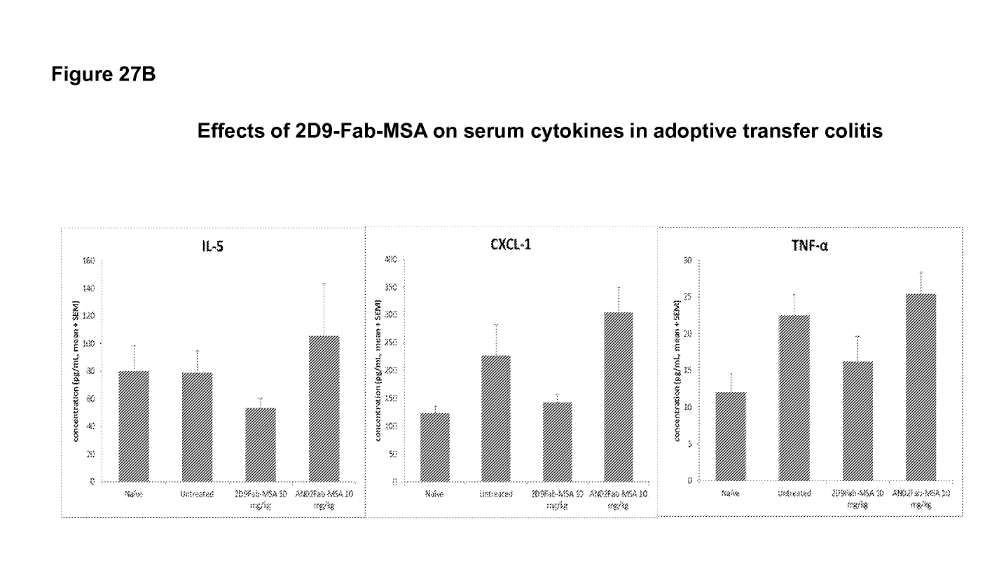

In addition to measuring the body weight of treated and untreated adoptive transfer colitis mice, a cytokine assay was conducted to determine changes in cytokine concentrations. As described in Example 16, the serum samples were prepared and the serum levels of nine Th1/Th2 cytokines (IFN-γ, IL-1b. IL-2, IL-4, IL-5, IL-10, IL-12, TNF-α, and CXCL-1) were analyzed. FIGS. 27A and B show that in animals with adoptive transfer colitis, with the exception of IL-5, serum levels of all the cytokines tested were increased. Particularly in these animals, the IFN-γ and IL-12 levels were four and two fold greater, respectively, than in naïve mice. Weekly IV administration of 2D9-Fab-MSA (10 mg/kg) to animals with adoptive transfer colitis reduced serum levels of IL-10, IL-12, CXCL-1 and TNF-α compared to the isotype control (AN02-Fab-MSA) treated group. Outliers beyond the range of 2×SD were excluded in the analysis based on Chauvenet's criterion (n=8-9).

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated humanized antibody or fragment thereof comprising an amino acid sequence selected from the group consisting of:
(a) an amino acid sequence that is at least 85% identical to any one of SEQ ID NOs:7-19 and an amino acid sequence that is at least 85% identical to any one of SEQ ID NOs:20-24; and
(b) the amino acid sequence of the VHCDR1, VHCDR2, VHCDR3 domains of any one of SEQ ID NOs:7-19 and the VLCDR1, VLCDR2, and VLCDR3 domains of any one of SEQ ID NOs:20-24
wherein said antibody or fragment thereof specifically binds a DR3 polypeptide.

2. The antibody or fragment thereof of claim 1 wherein the antibody or fragment thereof is a DR3 antagonist.

3. The antibody or fragment thereof of claim 1 wherein the antibody or fragment thereof is a DR3 agonist.

4. The antibody or fragment thereof of claim 1 wherein the antibody or fragment thereof is selected from the group consisting of:
(a) a whole immunoglobulin molecule;
(b) an scFv;
(c) a chimeric antibody;
(d) a Fab fragment;
(e) an Fab′ fragment;
(f) an F(ab′)2;
(g) an Fv; and
(h) a disulfide linked Fv.

5. The antibody or fragment thereof of claim 4 wherein the antibody or fragment thereof is a Fab fragment.

6. The antibody or fragment thereof of claim 5 wherein the antibody or fragment is fused to a human serum albumin polypeptide.

7. The antibody or fragment thereof of claim 6 wherein the human serum albumin polypeptide consists of the amino acid sequence of SEQ ID NO:4.

8. The antibody or fragment thereof of claim 4 wherein the antibody or fragment thereof is a whole immunoglobulin molecule.

9. The antibody or fragment thereof of claim 4 wherein the antibody or fragment thereof is an scFv.

10. The antibody or fragment thereof of claim 1 which further comprises a heavy chain immunoglobulin constant domain selected from the group consisting of:
(a) a human IgM constant domain;
(b) a human IgG1 constant domain;
(c) a human IgG2 constant domain;
(d) a human IgG3 constant domain;
(e) a human IgG4 constant domain; and
(f) a human IgA constant domain.

11. The antibody or fragment thereof of claim 10 wherein the heavy chain immunoglobulin constant domain is from human IgG1.

12. The antibody or fragment thereof of claim 11 wherein the heavy chain immunoglobulin constant domain is the CH1 from human IgG1.

13. The antibody or fragment thereof of claim 1 which further comprises a light chain immunoglobulin constant domain selected from the group consisting of:
(a) a human Ig kappa constant domain; and
(b) a human Ig lambda constant domain.

14. The antibody or fragment thereof of claim 13 wherein the light chain immunoglobulin constant domain is a human Ig kappa constant domain.

15. A method of treating, preventing or ameliorating a disease or disorder comprising identifying an animal suffering from said disease or disorder and administering the isolated humanized antibody or fragment thereof of claim 2 to said animal in an amount sufficient to treat, prevent or ameliorate said disease or disorder.

16. The method of claim 15, wherein the animal is a human.

17. The method of claim 15 wherein the disease or disorder is selected from the group consisting of:
(a) an inflammatory disease or disorder; and
(b) an autoimmune disease or disorder.

18. The method of claim 15 wherein the disease or disorder is selected from the group consisting of:
(a) Crohn's disease;
(b) Colitis;
(c) Inflammatory Bowel Disease;
(d) Arthritis;
(e) Multiple Sclerosis;
(f) Atherosclerosis;
(g) Allergic disorders
(h) Osteoporosis; and
(i) Bone cancer pain.

19. A method of inhibiting DR3 signaling in an animal comprising administering to said animal the isolated humanized antibody or fragment thereof in an amount sufficient to inhibit DR3 signaling.

20. The method of claim 19 wherein the isolated humanized antibody or fragment thereof inhibits TL1A-induced cytokine release.

21. The method of claim 19 wherein the isolated humanized antibody or fragment thereof inhibits T cell activation and/or proliferation.

22. The method of claim 19, wherein the isolated humanized antibody of fragment thereof inhibits activation of monocytes, macrophages, or dendritic cells.


SEQUENCE LISTING (28 sequences)

SEQ ID NO: 1 - DNA, 1254 bp, Homo sapiens
atggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtga

SEQ ID NO: 2 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 3 - DNA, 1755 bp, Homo sapiens
gatgcgcataaaagcgaagtggcgcatcgctttaaagatctgggcgaagaaaactttaaagcgctggtgctgattgcgtttgcgcagtatctgcagcagtgcccgtttgaagatcatgtgaaactggtgaacgaagtgaccgaatttgcgaaaacctgcgtggcggatgaaagcgcggaaaactgcgataaaagcctgcataccctgtttggcgataaactgtgcaccgtggcgaccctgcgcgaaacctatggcgaaatggcggattgctgcgcgaaacaggaaccggaacgcaacgaatgctttctgcagcataaagatgataacccgaacctgccgcgcctggtgcgcccggaagtggatgtgatgtgcaccgcgtttcatgataacgaagaaacctttctgaaaaaatatctgtatgaaattgcgcgccgccatccgtatttttatgcgccggaactgctgttttttgcgaaacgctataaagcggcgtttaccgaatgctgccaggcggcggataaagcggcgtgcctgctgccgaaactggatgaactgcgcgatgaaggcaaagcgagcagcgcgaaacagcgcctgaaatgcgcgagcctgcagaaatttggcgaacgcgcgtttaaagcgtgggcggtggcgcgcctgagccagcgctttccgaaagcggaatttgcggaagtgagcaaactggtgaccgatctgaccaaagtgcataccgaatgctgccatggcgatctgctggaatgcgcggatgatcgcgcggatctggcgaaatatatttgcgaaaaccaggatagcattagcagcaaactgaaagaatgctgcgaaaaaccgctgctggaaaaaagccattgcattgcggaagtggaaaacgatgaaatgccggcggatctgccgagcctggcggcggattttgtggaaagcaaagatgtgtgcaaaaactatgcggaagcgaaagatgtgtttctgggcatgtttctgtatgaatatgcgcgccgccatccggattatagcgtggtgctgctgctgcgcctggcgaaaacctatgaaaccaccctggaaaaatgctgcgcggcggcggatccgcatgaatgctatgcgaaagtgtttgatgaatttaaaccgctggtggaagaaccgcagaacctgattaaacagaactgcgaactgtttgaacagctgggcgaatataaatttcagaacgcgctgctggtgcgctataccaaaaaagtgccgcaggtgagcaccccgaccctggtggaagtgagccgcaacctgggcaaagtgggcagcaaatgctgcaaacatccggaagcgaaacgcatgccgtgcgcggaagattatctgagcgtggtgctgaaccagctgtgcgtgctgcatgaaaaaaccccggtgagcgatcgcgtgaccaaatgctgcaccgaaagcctggtgaaccgccgcccgtgctttagcgcgctggaagtggatgaaacctatgtgccgaaagaatttaacgcggaaacctttacctttcatgcggatatttgcaccctgagcgaaaaagaacgccagattaaaaaacagaccgcgctggtggaactggtgaaacataaaccgaaagcgaccaaagaacagctgaaagcggtgatggatgattttgcggcgtttgtggaaaaatgctgcaaagcggatgataaagaaacctgctttgcggaagaaggcaaaaaactggtggcggcgagccaggcggcgctgggcctg

SEQ ID NO: 4 - protein, 585 aa, Homo sapiens [sequence omitted]

SEQ ID NO: 5 - DNA, 1824 bp, Mus musculus
atgaaatgggtgacctttctgctgctgctgtttgtgagcggcagcgcgtttagccgcggcgtgtttcgccgcgaagcgcataaaagcgaaattgcgcatcgctataacgatctgggcgaacagcattttaaaggcctggtgctgattgcgtttagccagtatctgcagaaatgcagctatgatgaacatgcgaaactggtgcaggaagtgaccgattttgcgaaaacctgcgtggcggatgaaagcgcggcgaactgcgataaaagcctgcataccctgtttggcgataaactgtgcgcgattccgaacctgcgcgaaaactatggcgaactggcggattgctgcaccaaacaggaaccggaacgcaacgaatgctttctgcagcataaagatgataacccgagcctgccgccgtttgaacgcccggaagcggaagcgatgtgcaccagctttaaagaaaacccgaccacctttatgggccattatctgcatgaagtggcgcgccgccatccgtatttttatgcgccggaactgctgtattatgcggaacagtataacgaaattctgacccagtgctgcgcggaagcggataaagaaagctgcctgaccccgaaactggatggcgtgaaagaaaaagcgctggtgagcagcgtgcgccagcgcatgaaatgcagcagcatgcagaaatttggcgaacgcgcgtttaaagcgtgggcggtggcgcgcctgagccagacctttccgaacgcggattttgcggaaattaccaaactggcgaccgatctgaccaaagtgaacaaagaatgctgccatggcgatctgctggaatgcgcggatgatcgcgcggaactggcgaaatatatgtgcgaaaaccaggcgaccattagcagcaaactgcagacctgctgcgataaaccgctgctgaaaaaagcgcattgcctgagcgaagtggaacatgataccatgccggcggatctgccggcgattgcggcggattttgtggaagatcaggaagtgtgcaaaaactatgcggaagcgaaagatgtgtttctgggcacctttctgtatgaatatagccgccgccatccggattatagcgtgagcctgctgctgcgcctggcgaaaaaatatgaagcgaccctggaaaaatgctgcgcggaagcgaacccgccggcgtgctatggcaccgtgctggcggaatttcagccgctggtggaagaaccgaaaaacctggtgaaaaccaactgcgatctgtatgaaaaactgggcgaatatggctttcagaacgcgattctggtgcgctatacccagaaagcgccgcaggtgagcaccccgaccctggtggaagcggcgcgcaacctgggccgcgtgggcaccaaatgctgcaccctgccggaagatcagcgcctgccgtgcgtggaagattatctgagcgcgattctgaaccgcgtgtgcctgctgcatgaaaaaaccccggtgagcgaacatgtgaccaaatgctgcagcggcagcctggtggaacgccgcccgtgctttagcgcgctgaccgtggatgaaacctatgtgccgaaagaatttaaagcggaaacctttacctttcatagcgatatttgcaccctgccggaaaaagaaaaacagattaaaaaacagaccgcgctggcggaactggtgaaacataaaccgaaagcgaccgcggaacagctgaaaaccgtgatggatgattttgcgcagtttctggatacctgctgcaaagcggcggataaagatacctgctttagcaccgaaggcccgaacctggtgacccgctgcaaagataccctggcg

SEQ ID NO: 6 - protein, 584 aa, Mus musculus
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDTLA

SEQ ID NO: 7 - protein, 121 aa, Mus musculus
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYNCARRKDGNYYYAMDYWGQGTSVTVSS

SEQ ID NO: 8 - protein, 121 aa, Artificial Sequence
  humanized murine antibody
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYYCARRKDGNYYYAMDYWGQGTSVTVSS

SEQ ID NO: 9 - protein, 121 aa, Artificial Sequence
  humanized murine antibody
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYNCARRKDGNYYYAMDYWGQGTTVTVSS

SEQ ID NO: 10 - protein, 121 aa, Artificial Sequence
  humanized murine antibody
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYYCARRKDGNYYYAMDYWGQGTTVTVSS

SEQ ID NO: 11 - protein, 121 aa, Artificial Sequence
  humanized murine antibody
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYNCARRKDGNYYYAMDYWGQGTTVTVSS

SEQ ID NO: 12 - protein, 121 aa, Artificial Sequence
  humanized murine antibody
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYYCARRKDGNYYYAMDYWGQGTTVTVSS

SEQ ID NO: 13 - protein, 121 aa, Artificial Sequence
  humanized murine antibody
QVQLVESGGGLIQPGGSLRLSCAASGFTFTNYAMSWVRQARGQRLEWVSTITSGGSYIYYLDSVKGRFTISRDNSKSTLYMELSSLRSEDTAVYNCARRKDGNYYYAMDYWGQGTTVTVSS

SEQ ID NO: 14 - protein, 121 aa, Artificial Sequence
  humanized murine antibody
QVQLVESGGGLIQPGGSLRLSCAASGFTFTNYAMSWVRQARGQRLEWVSTITSGGSYIYYLDSVKGRFTISRDNSKSTLYMELSSLRSEDTAVYYCARRKDGNYYYAMDYWGQGTTVTVSS

SEQ ID NO: 15 - protein, 121 aa, Artificial Sequence
  humanized murine antibody
QVQLVQSGSELKKPGASVKVSCKASGFTFTNYAMSWVRQAPGKRLEWVSTITSGGSYIYYLDSVKGRFTISRENAKSTLYLQMNSLRTEDTALYNCARRKDGNYYYAMDYWGQGTTVTVSS

SEQ ID NO: 16 - protein, 121 aa, Artificial Sequence
  humanized murine antibody
QVQLVQSGSELKKPGASVKVSCKASGFTFTNYAMSWVRQAPGKRLEWVATITSGGSYIYYLDSVKGRFTISRENAKSTLYLQMNSLRTEDTALYYCARRKDGNYYYAMDYWGQGTTVTVSS

SEQ ID NO: 17 - protein, 121 aa, Artificial Sequence
  humanized murine antibody
EVQLLQSGAEVKKPGASVKVSCKASGFTFTNYAMSWVRQAPGQRLEWVATITSGGSYIYYLDSVKGRFTISRDNAKSTLHLQMNSLRAEDTAVYNCARRKDGNYYYAMDYWGQGTTVTVSS

SEQ ID NO: 18 - protein, 121 aa, Artificial Sequence
  humanized murine antibody
EVQLLQSGAEVKKPGASVKVSCKASGFTFTNYAMSWVRQAPGQRLEWVATITSGGSYIYYLDSVKGRFTISRDNAKSTLHLQMNSLRAEDTAIYYCARRKDGNYYYAMDYWGQGTTVTVSS

SEQ ID NO: 19 - protein, 121 aa, Artificial Sequence
  humanized murine antibody
EVMLLQSGAEVKKPGASVKVSCKASGFTFTNYAMSWVRQAPGQRLEWVATITSGGSYIYYLDSVKGRFTISRDNAKSTLHLQMNSLRAEDTAVYYCARRKDGNYYYAMDYWGQGTTVTVSS

SEQ ID NO: 20 - protein, 111 aa, Mus musculus
DIVLTQSPASLAVSLGQRATISCRASESVDSYGNSFIHWYQQKAGQPPKLLIYRASNLESGIPARFSGSGSRTDFTLTINPVEADDVATYYCQQSYEDPWTFGGGTKLEIK

SEQ ID NO: 21 - protein, 111 aa, Artificial Sequence
  humanized murine antibody
DIVLTQSPATLSLSPGERATLSCRASESVDSYGNSFIHWYQQKPGQPPKLLIYRASNLESGIPARFSGSGSRTDFTLTISSLEPEDFAVYYCQQSYEDPWTFGGGTKXEIK

SEQ ID NO: 22 - protein, 111 aa, Artificial Sequence
  humanized murine antibody
DIVLTQSPSSLSASVGDRVTITCRASESVDSYGNSFIHWYQQKPGQPPKLLIYRASNLESGIPARFSGSGSRTDFTLTISSLQPEDFATYYCQQSYEDPWTFGGGTKXEIK

SEQ ID NO: 23 - protein, 111 aa, Artificial Sequence
  humanized murine antibody
DIVLTQSPDFQSVTPKEKVTITCRASESVDSYGNSFIHWYQQKPGQPPKLLIYRASNLESGIPARFSGSGSRTDFTLTISSLEAEDAATYYCQQSYEDPWTFGGGTKXEIK

SEQ ID NO: 24 - protein, 111 aa, Artificial Sequence
  humanized murine antibody
DIVLTQTPLSLSVTPGQPASISCRASESVDSYGNSFIHWYQQKPGQPPKLLIYRASNLESGIPARFSGSGSRTDFTLKISRVEAEDVGVYYCQQSYEDPWTFGGGTKXEIK

SEQ ID NO: 25 - protein, 107 aa, Homo sapiens [sequence omitted]

SEQ ID NO: 26 - protein, 114 aa, Mus musculus
RTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 28 - protein, 102 aa, Mus musculus
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGP